(19) United States
(12) Patent Application Publication
(10) Pub. No.: US 2006/0002934 A1
(43) Pub. Date: Jan. 5, 2006

(54) Egf receptor antagonists in the treatment of gastric cancer

(76) Inventors: Birgit Luber, Munchen (DE); Margit Roswitha Fuchs, Munchen (DE); Heinz Hofler, Munchen (DE); Falko Fend, Kirchheim (DE); Armando Gamboa-Dominguez, Mexico (MX)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(21) Appl. No.: 10/514,351

(22) PCT Filed: May 14, 2003
(86) PCT No.: PCT/EP03/05057

§ 371 Date: Jul. 7, 2005

Related U.S. Application Data
(60) Provisional application No. 60/380,285, filed on May 15, 2002.

(30) Foreign Application Priority Data
Feb. 28, 2003 (EP) 03004524.9

(51) Int. Cl. A61K 39/395; A61K 48/00; A61K 31/353

(52) U.S. Cl. 424/145.1; 424/178.1; 514/44; 514/456

(57) ABSTRACT

The present invention relates to a use of (an) EGF receptor antagonist(s)/inhibitor(s) for the preparation of a pharmaceutical composition for the prevention, amelioration or treatment of gastric carcinomas, preferably for the prevention, amelioration or treatment of diffuse gastric carcinomas. Furthermore, the invention provides for a method for treating or for preventing gastric carcinomas, in particular diffuse gastric carcinomas comprising the administration of at least one EGF receptor antagonist/inhibitor to a subject in need of such a treatment or prevention.

The present invention relates to a use of (an) EGF receptor antagonist(s)/inhibitor(s) for the preparation of a pharmaceutical composition for the prevention, amelioration or treatment of gastric carcinomas, preferably for the prevention, amelioration or treatment of diffuse gastric carcinomas. Furthermore, the invention provides for a method for treating or for preventing gastric carcinomas, in particular diffuse gastric carcinomas comprising the administration of at least one EGF receptor antagonist/inhibitor to a subject in need of such a treatment or prevention.

Several documents are cited throughout text of this specification. Each of the documents cited herein (including any manufacturer's specifications, instructions, etc.) are hereby incorporated by reference.

Cancer is caused by a series of genomic chances leading directly or indirectly to disturbances of growth, differentiation and tissue integrity. Interestingly, for a plurality of cancers, mutations in cadherins have been described and in particular mutations in the E-cadherin/catenin complex have been postulated to be involved in the development of cancerous and/or tumorous diseases; see van Aken (2001), Virchows Arch. 439, 725-751.

For example, β-catenin mutations have been identified in colorectal carcinomas and melanoma cell lines revealed that β-catenin may function as an oncogene; see Korinek (1997), Science 275, 1784-1787; Morin (1997) Science 275, 1787-1790 or Rubinfeld (1997), Science 275, 1790-1792.

Furthermore, mutations in the cell adhesion molecule E-cadherin have been described in diffuse-type gastric and lobular breast cancers.

E-cadherin is a transmembrane receptor protein which mediates adhesive interactions between epithelial cells and regulates the organization of the actin cytoskeleton via its cytoplasmic binding partners, the catenins (Kemler, Trends Genet. 9 (1993), 317-321; Gumbiner, J. Cell Biol. 148 (2000), 399-404). E-cadherin acts as a suppressor of tumor invasion and is often downregulated or mutated in invasive and metastatic tumors (Birchmeier, Biochim. Biophys. Acta 1198 (1994), 11-26; Hirohashi, Am. J. Pathol. 153 (1998), 333-339). Somatic E-cadherin mutations were found in diffuse-type gastric carcinomas which are characterized by scattered tumor cell morphology and poor prognosis (Becker, Hum Mol. Genet. 2 (1993), 803-804; Becker, Cancer Res. 54 (1994), 3845-3852; Muta, Jpn. J. Cancer Res. 87 (1996), 834-848; Tamura, Jpn. J. Cancer Res. 87 (1996), 1153-1159) as well as in breast and ovarian carcinomas (Berx, Hum. Mutat. 12 (1998), 226-237). Germline E-cadherin mutations have been identified in families with diffuse-type gastric carcinoma (Guilford, Nature 392 (1998), 402-405; Gayther, Cancer Res. 58 (1998), 4086-4089; Richards, Hum. Mol. Genet. 8 (1999), 607-610; Keller, Am. J. Pathol. 155 (1999), 337-342). Recently, E-cadherin has been shown to be part of signal transduction pathways although the molecule lacks intrinsic enzymatic activity (Pece, J. Biol. Chem. 274 (1999), 19347-19351; Vleminckx, Bioessays 21 (1999), 211-220; Pece, J. Biol. Chem. 275 (2000), 41227-41233). Moreover, this adhesive receptor inhibits cell proliferation (Watabe, J. Cell Biol. 127 (1994), 247-256) and upregulates the cyclin-dependent kinase inhibitor p27 (St. Croix, J. Cell Biol. 142 (1998), 557-571). It is also suggested that E-cadherin plays an important role in outside-in signal transduction. For instance, E-cadherins activate MAP kinase through EGFR (Pece (2000), loc. cit.). Moreover, formation of E-cadherin-based adherens junctions triggers activation of the Pi 3-kinase-Akt/PKB pathway (Pece (1999), loc. cit.). So far, there is no evidence that cadherins signal by themselves, instead, associated signaling proteins are likely to mediate the effects.

Recently E-cadherin mutations were identified in diffuse-type gastric carcinomas (Becker (1993), loc. cit.; Becker (1994), loc. cit.) and the functional effects of the mutant molecules in cell culture were investigated (Handschuh, Oncogene 18 (1999), 4301-4312; Luber, Cell Adhes. Commun. 7 (2000), 391-408). The mutated E-cadherin molecules resulted in decreased cell adhesion and aggregation and enhanced migration of MDA-MB-435S mammary carcinoma cells and L929 fibroblasts in wound healing assays as compared to cells expressing wt E-cadherin. By these wound healing experiments, it was found that the region within the E-cadherin molecule responsible for its migration suppressor function resides within the linker region between domain 2 and 3. Mutant E-cadherin molecules were partially perinuclearly localized and caused perinuclear localization of its cytoplasmic binding partner β-catenin. An epithelial to mesenchymal transition upon expression of mutant E-cadherin was indicated morphologically.

E-cadherin, which is described as an invasion and tumor suppressor (Vleminckx, Cell 66 (1991), 107-119; Birchmeier (1994), loc. cit.; Hirohashi (1998), loc. cit.), acts also as a suppressor of cell growth, as demonstrated by cell culture and mouse experiments (Navarro, J. Cell. Biol. 115 (1991), 517-533; Watabe (1994), loc. cit.; Hermiston, J. Cell Biol. 129 (1995), 489-506; Miyaki, Oncogene 11 (1995), 2547-2552; Takahashi, Exp. Cell Res. 226 (1996), 214-222; Kandikonda, Cell Adhes. Commun. 4 (1996), 13-24; Stockinger, J. Cell Biol. 154 (2001), 1185-1196). E-cadherin has been reported to inhibit cell proliferation by a mechanism which includes upregulation of the cyclin-dependent kinase inhibitor p27^{KIP1} (St. Croix (1998), loc. cit.). In another study, it has been shown that E-cadherin regulates cell growth by modulating the β-catenin transcriptional activity (Stockinger (2001), loc. cit.).

E-cadherin plays also a role in protecting cells from apoptosis (Metcalfe, Bioessays 19 (1997), 711-720; Kantak, J. Biol. Chem. 273 (1998), 16953-16961; Day, J. Biol. Chem. 274 (1999), 9656-9664). The mechanism by which E-cadherin exerts its anti-apoptotic function is not yet known. Kantak and Kramer ((1998), loc. cit.) have suggested that interactions between E-cadherin and signalling molecules which are important for cellular survival are involved in the effect. For instance, E-cadherin has been shown to associate with the epidermal growth factor receptor which plays a role for cellular survival (Hoschuetzky, J. Cell Biol. 127 (1994), 1375-1380). On the other site, it has been suggested by Peluso, Biol. Signals Recept. 9 (2000), 115-121 that the cytoplasmic E-cadherin binding partner β-catenin is involved in the anti-apoptotic function of E-cadherin. The interaction of E-cadherin with the actin cytoskeleton is mediated by α- and β-catenin or plakoglobin (Ozawa, EMBO J. 8 (1989), 1711-1717; Nathke, J. Cell Biol. 125 (1994), 1341-1352). In addition to its role in cell adhesion, β-catenin is involved in the transcriptional regulation of the apoptosis-regulating genes c-myc (He, Science 281 (1998), 1509-1512) and c-jun (Mann, Proc. Natl. Acad. Sci. USA 96 (1999), 1603-1608).

The E-cadherin gene is frequently lost or mutated in tumors (Van Aken (2001), loc. cit.). Somatic E-cadherin mutations were found in diffuse-type gastric and lobular breast carcinomas, comprising missense, splice site and truncation mutations (Becker (1993), loc. cit.; Becker (1994), loc. cit.; Muta (1996), loc. cit.; Tamura (1996), loc. cit.; Berx (1998), loc. cit.). Inactivating germline E-cadherin mutations have been identified in families with diffuse type gastric carcinoma (Guilford (1998), loc. cit.; Gayther (1998), loc. cit.; Richards (1999), loc. cit.; Keller (1999), loc. cit.). Cloning and functional analysis of the tumor-associated E-cadherin mutations demonstrated that E-cadherin mutations influence regulatory cellular networks (Handschuh (1999), loc. cit.; Luber (2000), loc. cit.). E-cadherin mutations resulted in decreased cellular adhesion and increased cellular motility, alterations of the actin cytoskeleton, and an abnormal perinuclear localization of β-catenin.

Unfortunately, therapeutic approaches on the basis of the fact that E-cadherin is described as an invasion- and tumor-suppressor have not been available. Accordingly, there is a need in the art to develop means and methods for the effective therapeutic intervention in tumorous/cancerous disorders, for example in gastric carcinomas which relate to a modification/mutation in the wnt/β-catenin signal transduction pathway and/or a mutation in E-cadherin.

The solution to this technical problem is achieved by providing the embodiments characterized in the claims.

Therefore, the present invention relates to a use of (an) EGF receptor antagonist(s)/inhibitor(s) for the preparation of a pharmaceutical composition for the prevention, amelioration or treatment of gastric carcinomas.

As documented in the appended examples, the invention demonstrates surprisingly that transfectants expressing mutant E-cadherin show enhanced cell motility as compared to transfectants expressing wild-type E-cadherin. The appended examples document that the motility enhancement resulting from mutation in the β-catenin/E-cadherin complex is sensitive to the treatment with EGF-receptor inhibitors/antagonists.

EGFR (epidermal growth factor receptor) is known as a central element for signal transduction and diversification (Prenzel, Endocrine-related cancer 8 (2001), 11-31) and several mutations of said receptor have been identified in glioblastomas, non-small-cell lung carcinomas, breast cancer, ovarian carcinomas; see Prenzel (2001), loc. cit. Of particular interest was recently the fact that overexpression of EGFR and EGFR-family members, like HER2, has been attributed to human cancers and tumorous, in particular mammary-, bladder-, colon-, glioma-, non-small-cell lung-, pancreatic-, ovarian-, gastric-, lung-, solivary-, head and neck-tumors; see for review Penzel (2001), loc. cit. and Hong/Ullrich, Oncol. Biother. 1 (2000), 2-29. Recently, the use of antibodies directed against EGFR (Herceptin) have been employed in the treatment of breast cancer. Yet, it has to be stressed that these therapeutic approaches are employed when wild-type or mutated EGFR is overexpressed. Yasui and colleagues (Canc. Res. 48 (1988), 137-141 and Int. J. Cancer 41 (1988), 211-217) have observed elevated levels of EGFRs in gastric cancer. However, it is of note that no all gastric cancers show an elevated level of EGFR expression.

In context of the present invention it was surprisingly be found that gastric carcinomas, independently from their status of EGFR expression can be positively influenced by the use of EGF receptor antagonist(s) or inhibitor(s). Without being bound by theory, the examples of the present invention provide for evidence that EGF receptor activity may be upregulated by mutations in the β-catenin/E-cadherin complex and in particular by mutations in E-cadherin. Considering the appended examples, it is postulated that wild-type E-cadherin has an inhibitory function on EGFR activation and that mutant E-cadherin has lost this function.

Accordingly, it is shown that cancer cells comprising an E-cadherin/β-catenin complex mutation may be targeted and treated by the use of an EGF-receptor antagonist/inhibitor, independently from the expression status of EGFR on the cells. Therefore, as will be illustrated herein below, the use of EGFR antagonist(s)/inhibitor(s) for the treatment, prevention and/or amelioration of gastric carcinomas is also and in particular envisaged in gastric carcinomas which do not comprise an overexpression of EGFR. Here it is of note that an overexpression of EGFR in gastric carcinomas has merely been demonstrated in late stages of disease progression and in only 33 to 43% of investigated cases.

The term EGFR-receptor antagonist/inhibitor relates to inhibitors or antagonists of the EGF-receptor itself as well as to antagonists/inhibitors for phosphatidylinositol-3-kinase (PI-3 K, PI-3), as illustrated in the appended examples. Specific useful EGF-receptor antagonists/inhibitors will be described herein below and are illustrated in the appended examples.

In a preferred embodiment of the present invention, said gastric carcinoma to be treated is a diffuse gastric carcinoma. A treatment of diffuse gastric carcinomas is particular envisaged, since the treatment of said carcinomas with (an) EGFR antagonist(s)/inhibitor(s) can interfere with tumor cell dissemination. Accordingly, it is also preferred in the use of the present invention that the EGF receptor antagonist(s)/inhibitor(s) are employed for inhibiting the motility of tumor cells in a subject suffering from gastric carcinomas, in particular diffuse gastric carcinomas. Yet, the present invention does not exclude that the beneficial effect of EGF-receptor antagonist(s)/inhibitor(s) also lead to desired anti-proliferative events or desired apoptotic events.

As shown in the appended examples, E-cadherin mutations alter the metastatic behavior of tumor cells, accordingly it is speculated that this enhanced cell motility can be influenced and positively altered by the use of EGF receptor antagonists in vivo. It is also envisaged in context of this invention that the carcinoma cells of the patients suffering from gastric carcinomas, in partricular diffuse carcinomas, do not comprise an overexpression of EGFR. Here, it was surprisingly found that EGFR antagonist(s)/inhibitor(s) can influence the motility of cells comprising a mutation in the β-catenin/E-cadherin complex. This is in particular surprising, since these cells do not comprise an elevated level of EGF-receptor expression. Accordingly, here it was found that, without being bound by theory, (a) mutation(s) in E-cadherin may lead to an unphysiological activation or over-activation of EGFR. The expression level of EGFR in cells, tissues, tissue samples may be easily deduced by methods known in the art, which comprise, inter alia, the measurement of RNA (preferably mRNA) levels or protein levels. The person skilled in the art can, therefore, readily deduce whether a cell or tissue, for example cells or tissues derived from a biopsy, comprise an elevated level of EGFR. This may, inter alia, be done by Western- or Northern Blot analysis, for example in comparison to healthy, non-cancerous cells or tissues.

Preferably, in the use of the present invention, the cells derived from the gastric carcinomas or gastric diffuse carcinomas comprise at least one mutation in the β-catenin signal transduction pathway. In accordance with this invention, the term “β-catenin signal transduction pathway” relates to the “E-cadherin/catenin adhesion complex” and/or the wnt/β-catenin signal transduction pathway.

Recent evidence suggests that adherens junction molecules are involved in basic cellular mechanisms such as cell growth and differentiation (Klymkowsky, Cell 83 (1995), 5-8). Besides its role in cell adhesion, β-catenin has a signaling function in the wnt/wingless pathway which plays an important role in tumorigenesis and during embryonic development (Hunter, Cell 88 (1997), 333-346). The amount of β-catenin not associated with E-cadherin is regulated. Free β-catenin is phosphorylated by glycogen-synthetase kinase 3β (GSK-3β) in a multiprotein complex consisting of adenomatous polyposis coli protein (APC), conductin and axin (Zeng, Cell 90 (1997), 181-192; Behrens, Science 280 (1998), 596-599) and is thereby marked for degradation by the ubiquitin-proteasome pathway (Aberle, EMBO J. 16 (1997), 3797-3804). APC is mutated in most colorectal and in many gastric carcinomas. Non-functional APC or inhibition of GSK-3β activity by the activation of the wnt/wingless signaling cascade lead to the accumulation of free β-catenin in the cytoplasm. β-catenin then exerts its signaling function by binding to a member of the LEF-1/TCF family of transcription factors (Behrens, Nature 382 (1996), 638-642; Huber, Mech. Dev. 59 (1996), 3-10; Molenaar, Cell 86 (1996), 391-399). Subsequently, this complex is translocated to the nucleus where it activates gene expression, for example c-myc (He (1998), loc. cit.) and cyclin D (Mann (1999), loc. cit.; Shtutman, Proc. Natl. Acad. Sci. USA 96 (1999), 5522-5527; Tetsu, Nature 398 (1999), 422-426), gene expression.

Accordingly, the present invention provides for the use of EGF receptor antagonist(s)/inhibitor(s) in the preparation of a medicament for the treatment or prevention of gastric carcinomas, whereby cells derived from said carcinoma comprise at least one mutation in β-catenin, GSK-3β, APC, or a member of the LEF-1/TCF family. Such mutations are known in the art and comprise, inter alia, β-catenin mutations in gastric carcinomas (see Sasaki, Tumour Biol. 22 (2001), 123-130; Woo, Int. J. Canc. 95 (2001), 108-113 or Tong, Canc. Lett. 163 (2001), 125-130) or APC mutations in gastric carcinomas as described in Ming, Gastric Canc. 1 (1998), 31-50.

In a preferred embodiment of the present invention cells of gastric carcinomas to be treated comprise a mutation in E-cadherin. Such mutations have been described in the art and comprise, inter alia and most frequently, in frame deletions of exon 8 or 9 (Handschuh (1999), loc. cit.). Yet, these mutations may also comprise inversions, deletions, additions, substitutions, duplications etc. The mutations may comprise germ line as well as somatic mutations.

As discussed herein above, the cell adhesion molecule E-cadherin mediates adhesive interactions between epithelial cells and influences the organization of the actin cytoskeleton by binding to catenins which serve as bridging molecules (Kemler (1993), loc. cit.; Gumbiner (2000), loc. cit.). E-cadherin has been identified as a suppressor of tumor invasion and is often downregulated or mutated in invasive and metastatic tumors (Birchmeier (1994), loc. cit.; Hirohashi (1998), loc. cit.). Previous results suggest a causal relationship between somatic E-cadherin mutations and the development and progression of diffuse type gastric carcinoma (Becker (1994) loc. cit.; Becker, Hum. Mut. 13 (1998), 171; Becker, Hum. Mutat. 12 (1998), 226-327; Muta (1996), loc. cit.; Tamura (1996), loc. cit.; Berx (1998), loc. cit., Machado, Lab. Invest. 79 (1999), 459-465). According to Laurén (Acta Pathol. Microbiol. Scand. 64 (1965), 31-49), gastric carcinomas are classified into diffuse-type gastric carcinomas with infiltrating, non-cohesive tumor cells and intestinal carcinomas with cohesive, glandular-like cell groups. Somatic mutations within the E-cadherin gene have been identified in 50% of the investigated diffuse-type gastric carcinoma and lymph-node metastasis derived thereof, but not in intestinal-type gastric carcinoma (Becker (1994), loc. cit.). Accordingly, there is a correlation between the histopathologic classification of diffuse-type gastric carcinoma and the molecular biological finding of abnormal (“mutated”) E-cadherin variants. Also, germline E-cadherin mutations have been identified in families with diffuse-type gastric carcinoma (Gayther (1998), loc. cit.; Guilford (1998), loc. cit.; Keller (1999), loc. cit., Richards (1999), loc. cit.).

A major function of the cell-to-cell adhesion molecule E-cadherin is the maintenance of cell adhesion and tissue integrity. E-cadherin deficiency in tumors leads to changes in cell morphology and motility, so that E-cadherin is considered to be a suppressor of invasion. The functional consequences of three tumor-associated gene mutations that affect the extracellular portion of E-cadherin were investigated: in-frame deletions of exons 8 or 9 and a point mutation in exon 8, as they were found in human gastric carcinomas. Human MDA-MB-435S breast carcinoma cells and mouse L fibroblasts were stably transfected with the wild-type and mutant cDNAs, and the resulting changes in localization of E-cadherin, cell morphology, strength of calcium-dependent aggregation as well as cell motility and actin cytoskeleton organization were studied. It was found that cells transfected with wild-type E-cadherin showed an epitheloid morphology, while all cell lines expressing mutant E-cadherin exhibited more irregular cell shapes. Cells expressing E-cadherin mutated in exon 8 showed the most scattered appearance, whereas cells with deletion of exon 9 had an intermediate state. Mutant E-cadherins were localized to the lateral regions of cell-to-cell contact sites. Additionally, both exon 8-mutated E-cadherins showed apical and perinuclear localization, and actin filaments were drastically reduced. MDA-MB-435S cells with initial calcium-dependent cell aggregation exhibited decreased aggregation and, remarkably, increased cell motility, when mutant E-cadherin was expressed. Therefore, it can be concluded that E-cadherin mutations may not simply affect cell adhesion but act in a trans-dominant-active manner, i.e. lead to increased cell motility. Results presented in the appended examples demonstrate that E-cadherin mutations, affecting, inter alia, exons 8 or 9, are the cause of multiple morphological and functional disorders and could induce the scattered morphology and the invasive behaviour of diffuse type-gastric carcinomas.

As shown in the appended examples, motility enhancement by mutant E-cadherin derived from gastric carcinomas was investigated by time-lapse laser scanning microscopy. The motility increasing activity of mutant E-cadherin was blocked by application of pharmacological inhibitors of epidermal growth factor receptor (EGFR) and phosphatidylinositol (PI) 3-kinase. Therefore, the use of EGF receptor antagonists for the treatment of gastric cancer patients, in particular of patients suffering from diffuse gastric carcinomas is suggested in this invention. As pointed out herein below, the treatment regimes provided herein, i.e. the use of EGFR antagonist(s)/inhibitor(s) is particularly desired where metastatic potential of gastric carcinoma cells, in particular diffuse gastric carcinoma cells needs to be suppressed.

In a particular preferred embodiment of the present invention the EGFR antagonist(s)/inhibitor(s) is (are) to be used in the preparation of a medicament for the treatment of gastric cancer, when the E-cadherin mutation of the cell(s) of the gastric carcinoma is selected from the group consisting of a full or partial deletion of exon 8, a full or partial deletion of exon 9, a full or partial deletion of exon 10 or one or more point mutations.

Particular E-cadherin mutations are shown in the following table:

Mutations of the E-cadherin gene in gastric carcinoma or cell lines derived thereof (until 1999)

| | | | | | Histological | | |
|---|---|---|---|---|---|---|---|
| | | | | Predicted protein | tumor type | | Type of |
| Codon | Exon | Intron | Nucleotide change | change | or cell line | Reference | mutation |
| 123 | 3 | — | 367C→T | H123Y | diffuse | Becker et al, 1998 | somatic |
| 157 | 4 | — | 469del63 (C) | In frame deletion | diffuse | Becker et al, 1998 | somatic |
| 193 | 5 | — | 578 A→C | T193P missense | diffuse | Muta et al, 1996 | somatic |
| 204 | 5 | — | 612del 9 (C) | In-frame deletion | diffuse | Becker et al, 1998 | somatic |
| 245 | 6 | — | 732del 9 (C) | In-frame deletion | diffuse | Becker et al, 1998 | somatic |
| 275 | 7 | — | 823del10 + 1del8 | In-frame deletion | MKN45 | Oda et, 1994 | somatic |
| 334 | 7 | — | 1000G→C | missense | diffuse | Machado et al, 1999 | somatic |
| 336 | 7 | — | 1008G→A | Frameshift (E336E) | KatolII | Oda et, 1994 | somatic |
| 336 | 7 | — | 1008G→T | Frameshift | diffuse | Guilford et al, 1998 | germline |
| | | | | (E336D) | | | |
| 336 | 7 | — | 1008delG | In-frame deletion | SRCC St | Tamura et al, 1996 | somatic |
| — | — | 7 | 1009-1del3 | In-frame deletion | diffuse | Becker et al, 1994 | somatic |
| — | — | 7 | 1009-1G→A | In-frame deletion | diffuse | Machado et al, 1999 | somatic |
| — | — | 7 | 1009-1G→A | In-frame deletion | mixed | Machado et al, 1999 | somatic |
| — | — | 7 | 1009-1G→A | In-frame deletion | mixed | Machado et al, 1999 | somatic |
| 337 | 8 | — | 1009del129(C) | In-frame deletion | diffuse | Becker et al, 1994 | somatic |
| 337 | 8 | — | 1009del129(C) | In-frame deletion | diffuse | Becker et al, 1998 | somatic |
| 337 | 8 | — | 1010insA | In-frame deletion | SRCC St | Tamura et al, 1996 | somatic |
| 343 | 8 | — | 1027delC | Frameshift | diffuse | Machado et al, 1999 | somatic |
| 347 | 8 | — | 1040C→T | A347V missense | diffuse | Machado et al, 1999 | somatic |
| 353 | 8 | — | 1057G→T | E353 stop | diffuse | Machado et al, 1999 | somatic |
| 365 | 8 | — | 1093G→C | V365L missense | mixed | Machado et al, 1999 | somatic |
| 369 | 8 | — | 1105A→G | N369D missense | diffuse | Machado et al, 1999 | somatic |
| 370 | 8 | — | 1109A→C | D370A missense | diffuse | Becker et al, 1994 | somatic |
| 370 | 8 | — | 1108G→C | D370H missense | diffuse | Machado et al, 1999 | somatic |
| 370 | 8 | — | 1108G→C | D370H missense | mixed | Machado et al, 1999 | somatic |
| — | — | 8 | 1138-11del28 | In-frame deletion | SRCC St | Muta et al, 1996 | somatic |
| — | — | 8 | 1138-11del21 | In-frame deletion | SRCC St | Muta et al, 1996 | somatic |
| 380 | — | — | 1138del183 (C) | In-frame deletion | diffuse | Becker et al, 1994 | somatic |
| 380 | — | — | 1138del183 (C) | In-frame deletion | mixed | Becker et al, 1994 | somatic |
| 380 | — | — | 1138del183 (C) | In-frame deletion | STO46 | Becker et al, 1994 | somatic |
| 400 | 9 | — | 1198del3 | In-frame deletion | diffuse | Tamura et al, 1996 | somatic |
| 400 | 9 | — | 1198G→T | D400Y missense | mixed | Machado et al, 1999 | somatic |
| 402 | 9 | — | 1204G→A | D402N missense | diffuse | Machado et al, 1999 | somatic |
| 418 | 9 | — | 1252del18 | In-frame deletion | diffuse | Tamura et al, 1996 | somatic |
| — | — | 9 | 1320 + 1delG | In-frame deletion | diffuse | Tamura et al, 1996 | somatic |
| — | — | 9 | 1320 + 1G→T | In-frame deletion | diffuse | Becker et al, 1994 | somatic |
| — | — | 9 | 1320 + 1G→C | In-frame deletion | diffuse | Becker et al, 1994 | somatic |
| — | — | 9 | 1320 + 26A→T | Not determined | SRCC St | Muta et al, 1996 | somatic |
| 441 | 10 | — | 1321del69 (C) | In-frame deletion | diffuse | Becker et al, 1994 | somatic |
| 463 | 10 | — | 1387G→C | E463Q missense | diffuse | Becker et al, 1994 | somatic |
| 470 | 10 | — | 1409C→T | T470I missense | diffuse | Guilford et al, 1998 | germline |
| 473 | 10 | — | 1418T→A | V473N missense | diffuse | Becker et al, 1994 | somatic |
| 479 | 10 | — | 1436A→G | D479G missense | diffuse | Machado et al, 1999 | somatic |
| 581 | 12 | — | 1742T→C | L581P missense | diffuse | Machado et al, 1999 | somatic |
| 598 | 12 | — | 1793G→A (C) | R598Q missense | STO44 | Becker et al, 1998 | somatic |
| 699 | 13 | — | 2095C→T | Q699stop | diffuse | Guilford et al, 1998 | germline |
| 794 | 15 | — | 2382insC | frameshift | diffuse | Guilford et al, 1998 | germline |
| | | | (stretch of C) | | | | |

(C) the mutation has been identified only at the cDNA level
difffuse diffuse-type gastric carcinoma
mixed mixed type gastric carcinoma with diffuse and intestinal components
SRCC St signet ring cell carcinoma of stomach

Besides the specific mutations of E-cadherin defined herein above, further mutations of E-cadherin are envisaged in context of this invention. For example mutations in the non-coding region of the E-cadherin gene, e.g. in the promoter region. The promoter structure of E-cadherin is known in the art, see, inter alia, van Aken (2001), loc. cit. and references therein.

As pointed out above and illustrated in the appended examples, the mutations described herein and in particular E-cadherin mutations lead to an enhanced cellular motility of cancer cells which can be successfully inhibited and positively influenced by inhibitors of EGFR. Accordingly, the present invention provides for uses and medical methods where (an) EGF receptor antagonist(s)/inhibitor(s) is/are employed in the medical intervention of gastric carcinomas, preferably diffuse carcinomas, whereby said inhibitor leads in a preferred embodiment to an inhibition of motility/metastasis formation of the gastric carcinoma cells. The present examples appended hereto provide for in vitro tests for testing the motility of cancer cells. These tests may also be employed on cells derived from biopsies. Furthermore, a model (in particular a mouse model) is provides which allows for investigating and verifying inhibitors/antagonists of EGF receptor/receptor functions in their potential for inhibiting tumor formation and/or metastasic processes in gastric cancer cells, in particular gastric cancer cells comprising (an) E-cadherin mutation(s).

In accordance with the appended examples and the description herein above, the present invention also relates to a method for the treatment of gastric carcinomas as defined herein above, and in particular of gastric diffuse carcinomas comprising mutations of E-cadherin, whereby said method comprises the administration of (an) EGF receptor antagonist(s)/inhibitor(s) to a subject in need of such a treatment. Most preferably said subject is a human subject. The embodiments herein above for the use of the present invention apply, mutatis mutantis, for the here described method.

It is also envisaged that said method of the invention comprises the co-therapy with other anti-cancer drugs, like, inter alia, cisplatin 5-fluorouracil, mitomycin, thiotepa, taxol or etoposid. Furthermore, it is envisaged that the method of the invention is employed in a co-therapy approach with other anti-cancer/anti-tumor treatments, like radiotherapy. Additionally, it is envisaged that the EGFR antagonist(s)/inhibitor(s) are employed in co-therapy approaches with antibodies directed against specific mutations of E-cadherin, like antibodies or derivatives or fragments thereof, directed against del8 and/or del9 mutatns of E-cadherin. It is in particular envisaged that EGFR antagonist(s)/inhibitor(s) are used in accordance with this invention in co-therapy approaches for gastric cancers and/or for co-therapy approaches to inhibit metastatic progression of gastric carcinomas. A systematic overview of currently available therapies and chemotherapy effects in gastric cancer is given in Janunger, Acta Oncol. 40 (2001), 309-326.

The invention also relates to the uses and the method as described herein, whereby the EGF receptor antagonist(s)/inhibitor(s) is (are) selected from the group consisting of an anti-EGFR antibody or a derivative or a fragment thereof, EGF-toxin or immunotoxin conjugate, antisense oligonucleotides specific for EGFR nucleic acid molecules, siRNA and/or RNAI directed against EGFR ribozymes specific for EGFR nucleic acid molecules.

Anti-EGFR-antibodies or derivatives or fragments thereof are known in the art. Such antibodies comprise monoclonal as well as polyclonal antibodies and derivatives or fragments thereof. Derivatives of such EGFR-antibodies may comprise humanized or CDR-grafted antibodies, as well as antibody fragments which specifically interact with EGFR and lead to an inhibition of EGFR and/or its mediated signal transduction pathway. Such antibody fragments comprise, inter alia, Fab-, F(ab)₂- or F(abc)-fragments. Furthermore, the use of single-chain antibodies or bispecific antibodies or antibody constructs is envisaged in the use and the methods of the present invention.

The ligand-toxins or immunotoxin-conjugates to be employed in accordance with this invention comprise, inter alia, EGF-, NRG- or TGF-α-conjugates which are covalently or non-covalently linked to toxic substances, like Pseudomonas exotoxin A or DAB389 or to (broad spectrum) tyrosine kinase inhibitors, like genistein.

The tyrosine kinase inhibitors to be employed as EGFR antagonist(s)/inhibitor(s) in accordance with this invention may comprise tyrphostin AG1478, ZD-1839, OSI-774, PKI-166, PD158780, CPG 59326, C₁₋₁₀₃₃.

As mentioned herein above, also siRNAs/RNAis (iRNAs), antisense molecules and ribozymes directed against nucleic acid molecules encoding EGFR are envisaged as EGFR antagonist(s)/inhibitor(s) for the use and the method of the present invention. These antisense molecules may not only comprise EGFR-antisense molecules and constructs but also TGF-α-antisense molecules and constructs. Such constructs are particularly useful in gene therapy approaches.

The above-mentioned antagonist/inhibitor of EGF receptor may also be a co-suppressive nucleic acid.

An siRNA approach is, for example, dislosed in Elbashir ((2001), Nature 411, 494-498)). It is also envisaged in accordance with this invention that for example short hairpin RNAs (shRNAs) are employed in accordance with this invention as pharmaceutical composition. The shRNA approach for gene silencing is well known in the art and may comprise the use of st (small temporal) RNAs; see, inter alia, Paddison (2002) Genes Dev. 16, 948-958.

As mentioned above, approaches for gene silencing are known in the art and comprise “RNA”-approaches like RNAi (iRNA) or siRNA. Successful use of such approaches has been shown in Paddison (2002) loc. cit., Elbashir (2002) Methods 26, 199-213; Novina (2002) Mat. Med. Jun. 3, 2002; Donze (2002) Nucl. Acids Res. 30, e46; Paul (2002) Nat. Biotech 20, 505-508; Lee (2002) Nat. Biotech. 20, 500-505; Miyagashi (2002) Nat. Biotech. 20, 497-500; Yu (2002) PNAS 99, 6047-6052 or Brummelkamp (2002), Science 296, 550-553. These approaches may be vector-based, e.g. the pSUPER vector, or RNA polIII vectors may be employed as illustrated, inter alia, in Yu (2002) loc. cit.; Miyagishi (2002) loc. cit. or Brummelkamp (2002) loc. cit.

Further EGFR antagonist(s)/inhibitor(s) are known in the art and partially described in Modi, Current Oncology Reports 4 (2002), 47-55. Also an EGF-vaccine, as described in Modi (2002), loc. cit. is envisaged as an EGF antagonist/inhibitor to be used and employed in the uses and methods of the present invention.

### THE FIGURES SHOW

FIG. 1: Enhancement of cell motility by mutant E-cadherin.
(A) Schematic drawing showing the exon structure of E-cadherin-cDNA used for transfection of MDA-MB-435S cells. The point mutation in exon 8 changes the codon GAT (position 370; clone HSECAD, Genbank/EMBL Z13009) to GCT (aspartic acid to alanine), thereby mutating the putative calcium binding site DTND to DTNA. Abbreviations: TM: transmembrane domain, cyto: cytoplasmic domain, EC1-5: extracellular domain 1 to 5.
(B) Non-transfected MDA-MB-435S cells (MDA) and transfected MDA-MB-435S cells expressing wt or mutant (del 9, del 8, p8) E-cadherin-cDNAs were plated on collagen I-coated glass plates. Phase contrast images were taken every 3 min for 7 h with a laser scanning microscope equipped with a temperature and CO₂-controlled incubation chamber, starting 2 h after plating. The percentage of motile cells was determined by counting cells of a microscopic field which moved completely out of the initial area within the time of the record. Only attached nondividing cells were analyzed that did not leave the observation field during the period of investigation. Each bar represents the mean + SD of at least three independent experiments. A total of at least 60 cells was investigated for each cell line in at least threee independent experiments.
(C) Semiautomatic tracing of cell nuclei allowed determination of the individual speed of 60 cells for each cell line derived from at least three different microscopic fields. Calculation of the cell speed is based on the division of the displacement of an individual cell divided by the total time of recording. The bars represent the range between the minimal and the maximal cell speed in a population of 60 cells per cell line derived from three independent microscopic fields. Arrowheads indicate the median of cell speeds.

FIG. 2: Mutant E-cadherin affects the style of cell movement.
Cells were plated on type I collagen-coated dishes and examined by laser scanning microscopy. Individual cells attached on collagen I coated dishes were tracked for the indicated time points, starting 2 h after plating. Shown are MDA-MB-435S cells expressing wt E-cadherin (A) or del 8 E-cadherin (B), one motile cell is marked by an arrow. The bars represent 50 μm.

FIG. 3: Mutant E-cadherin induces random cell movement.
MDA-MB-435S cells expressing wt or del 8 E-cadherin were recorded for 7 h starting 2 h after plating on collagen I-coated dishes. Cell nuclei were traced semiautomatically using the Zeiss LSM software. Shown are the paths of 10 randomly chosen cells within a microscopic field (460×460 μm).

FIG. 4: Cell adhesion and motility of MDA-MB-435S cells expressing wt or del 8-E-cadherin-cDNA on different ECM proteins.
(A) Cells were seeded on poly-lysine, collagen I, fibronectin, or vitronectin-coated plates and allowed to adhere to the different purified ECM proteins for 20 min at 37° C. and 5% CO₂ in a cell culture incubator. Non-adherent cells were removed after 20 min and cell viability of the residual attached cells was determined as described for XTT-cell proliferation and viability assay. Quadruplicate determinations were performed for each value and the mean +SD is shown. The figure shows one representative of three independent experiments.
(B) Cells were plated on glass plates coated with poly-lysine, collagen I, fibronectin or vitronectin and cell motility was analyzed as described in legend to FIG. 1B. Each bar represents the mean+SD of at least three independent experiments.
(C) Shown are the medians and the ranges of cell speeds, determined as described in legend to FIG. 1C.
(D) Flow cytometric analysis was carried out on non-transfected, wt and mutant (del 9, del 8, p8) E-cadherin expressing cells stained with monoclonal antibodies to α1 and β1 integrins and DTAF-conjugated anti-mouse IgG or isotype control. Similar results were obtained for all tested cell lines, only the results for wt and del 8 E-cadherin expressing cells are shown.

FIG. 5: Influence of PD 98059, LY 294002, Tyrphostin AG 1478 and EGF on cell motility.
Comparison of the percentage of motile cells (A), medians and ranges of cell speeds (B) of human MDA-MB-435S transfected with wt or del 8 E-cadherin-cDNA in the presence of PD 98059, LY 294002, Tyrphostin AG 1478, and EGF. Cells were plated onto a collagen I matrix and traced for 7 h under a phase-contrast microscope as described in the legend to FIG. 1. Only attached non-dividing cells were analyzed. Each bar represents the mean±SD of three independent experiments. (C) Shown are MDA-MB-435S cells expressing wt or del 8E-cadherin plated on glass plates coated with collagen I in the presence of PD 98059, LY 294002, Tyrphostin AG 1478 and EGF 2 h after plating. Concentrations: PD 98059: 50 μM; LY 294002: 40 μM; Tyrphostin AG 1478: 6,3 μM; EGF: 100 ng/ml. The bar represents 50 μm.

FIG. 6: Detection of Pi 3-kinase activity.
PI 3-kinase activity was assayed in anti-PI 3-kinase p85 immunoprecipitates from cell lysates of non-transfected (MDA), wt, del 9, del 8, or p8 E-cadherin-cDNA expressing MDA-MB-435S cells. The reaction product ³³P labeled phosphatidylinositol 3-phosphate (PI3P) is indicated. As a negative control, LY 294002 was included into the reaction. As a positive control, serum-starved cells were treated with EGF for 2 min. Concentrations: LY 294002: 10 μM; EGF: 100 ng/ml.

FIG. 7: Detection of activated and total MAP kinase and Akt/PKB levels.
Expression levels and activities of MAP kinases p44 and p42 (A) and Akt/PKB (B) were investigated by immunoblot analysis of extracts from non-transfected (MDA), wt, del 9, del 8, or p8 E-cadherin-cDNA expressing MDA-MB-435S cells using the respective antibodies. (A) Phospho-p44/42 MAP kinase polyclonal antibody detects activated p44/42 MAP kinase phosphorylated at threonine 202 and tyrosine 204. p44/42 MAP kinase polyclonal antibody detects total MAP kinase levels. Equal amounts of whole cell lysates prepared from uncoated plates were used in each lane. (B) Phospho-Akt polyclonal antibody detects Akt1, Akt2 and Akt3 when phosphorylated at Ser 473. Akt kinase polyclonal antibody detects total Akt kinase levels. Equal amounts of whole cell lysates prepared from uncoated plates were used in each lane.

FIG. 8: Comparison of the proliferation rate of parental, wt and mutant E-cadherin expressing MDA-MB-453S cells.
Non-transfected, wt and mutant E-cadherin expressing MDA-MB-435S cells were seeded into 96-well plates and assayed for proliferation and viability for 72 h. An increase in number of living cells results in an increase in the activity of mitochondrial enzymes which correlates with the amount of formazan formed by cleavage of XTT tetrazolium salt. Formazan formation was measured spectrophotometrically at 450 nm at the indicated time points. The absorbance value obtained when culture medium without cells was assayed was subtracted from the values obtained with cells. Quadruplicate determinations were performed for each time point.

The bars indicate the standard deviation. Shown is one representative of three independent experiments.

FIG. 9: Sensitivity profiles of E-cadherin expressing MDA-MB-435S transfectants to the chemotherapeutic drug cisplatin.
(A) Cells were seeded in 96-well plates and treated with increasing concentrations of cisplatin. Cell survival after a 48 h treatment with cisplatin at the indicated doses was determined by XTT assay. The results are expressed as percentage of absorbance values obtained with treated cells as compared to untreated cells. Quadruplicate determinations were performed for each concentration. The bars indicate the standard deviation.
(B) Cells were seeded in 6-well plates and treated with increasing concentrations of cisplatin. The number of colonies was determined after fixation and staining with Diff Quick Reagent after 7 days. The percentage of colony formation after exposure to cisplatin was determined in relation to non-treated cells. Triplicate determinations were performed for each concentration. The bars indicate the standard deviation.

FIG. 10: Sensitivity profiles of E-cadherin expressing MDA-MB-435S transfectants to the chemotherapeutic drug etoposide.
(A) Cells were seeded in 96-well plates and treated with increasing concentrations of etoposide. Cell survival after a 48 h treatment with etoposide at the indicated doses was determined by XTT assay as described in FIG. 9A.
(B) Cells were seeded in 6-well plates and treated with increasing concentrations of etoposide. The number of colonies was determined as described in FIG. 9 B.

FIG. 11: Sensitivity profiles of E-cadherin expressing MDA-MB-435S transfectants to the chemotherapeutic drug 5-FU.
(A) Cells were seeded in 96-well plates and treated with increasing concentrations of 5-FU. Cell survival after a 48 h treatment with 5-FU at the indicated doses was determined by XTT assay as described in FIG. 9A.
(B) Cells were seeded in 6-well plates and treated with increasing concentrations of 5-FU. The number of colonies was determined as described in FIG. 9B.

FIG. 12: p53 mutation status of parental, wt and mutant E-cadherin expressing MDA-MB-435S transfectants.
(A) The double-peak in the DHPLC chromatogram of exon 8 shows the p53 mutation G266E. Similar chromatographic patterns were observed for the parental, as well as wt and mutant E-cadherin expressing MDA-MB-435S cells. A gastric carinoma tissue without p53 mutation served as negative control.
(B) Mutation analysis of p53 indicates the presence of a wt and a mutant p53 allele in MDA-MB-435S cells. Shown is the p53 missense mutation G>A at nucleotide position 797.

FIG. 13: p53 expression level of parental, wt and mutant E-cadherin expressing MDA-MB-435S transfectants.

The p53 expression level was analyzed by immunoblot analysis of lysates from non-transfected (MDA), wt, del 9, del 8, or p8 E-cadherin-cDNA expressing MDA-MB-435S cells using monoclonal antibody p53 (AB-6). Equal amounts of whole cell lysates were used in each lane.

FIG. 14: Detection of total and activated p38 kinase levels.
The expression levels and activity of p38 MAP kinase were investigated by immunoblot analysis of extracts from non-transfected (MDA), wt, del 9, del 8, or p8 E-cadherin-cDNA expressing MDA-MB-435S cells using the respective antibodies which detect either total p38 kinase levels or phospho-p38 MAP kinase.

FIG. 15: Expression of wt and mutant E-cadherin-cDNA in MDA-MB-435S cells.
Flow cytometric analysis was carried out on parental, wt and mutant (del 9, del 8, p8) E-cadherin expressing cells stained with monoclonal antibodies to E-cadherin and DTAF-conjugated anti-mouse IgG.

FIG. 16: Tumorigenicity and lung metastasis of parental, wt and mutant E-cadherin expressing MDA-MB-435S cells in SCID mice.
MDA-MD-435S cells (parental, n=5), wt (n=5), del 8 (n=5), del 9 (n=5), and p8 (n=6) E-cadherin expressing cells were injected in the mammary fat pads of SCID mice. (A) Tumor growth was measured twice per week. At day 38, two mice per cell line were sacrificed to determine whether metastasis formation had already occured. (B) At day 46, the remaining animals were sacrificed and tumor volumes were calculated.

FIG. 17: Immunohistochemical study of E-cadherin and cytokeratin in primary tumors and metastases.
(A) Neoplastic cells of tumors derived from parental MDA-MB-435S cells show complete E-cadherin negativity. Epithelial mice cells from residual adnexa of the skin serve as internal staining control (arrow).
(B) Tumors obtained after transplantation of wt E-cadherin expressing MDA-MB-435S cells show membranous E-cadherin staining in 20% of the neoplastic cells. (C) E-cadherin expression was detectable only in 5% of the neoplastic cells of tumors induced after injection of del 8 E-cadherin expressing MDA-MB-435S cells. In contrast, much higher positivity was detectable in tumors with del 9 E-cadherin mutation (D, 80%) and p8 E-cadherin mutation (E, 80%). (F) A representative lung metastasis of an animal transplanted with del 9 E-cadherin expressing MDA-MB-435S cells is shown. The neoplastic cells show no E-cadherin reactivity while alveolar cells reveal strong membranous E-cadherin staining (arrow). Original magnification: (A, C, E)×300, (B)×200, (D, F)×400.

FIG. 18: Immunohistochemical study of del 8 and del 9 E-cadherin mutations in cell lines and corresponding xenograft tumors.
(A) The cell line with del 8 E-cadherin mutation shows a strong membranous positivity with del 8-specific antibody in the majority of the tumor cells. (B) In contrast, the mice tumors derived from this cell line reveal rare cells with membranous expression (arrow). The cell line with del 9 E-cadherin (C) and the tumor derived from this cell line (D) shows a membranous staining in the majority of the tumor cells with the del 9-specific antibody. Original magnification: (A, D)×400; (B, C)×640.

FIG. 19: Immunohistochemical study of MiB1 in primary tumors.
(A) The tumor derived from cells expressing wt E-cadherin show nuclear MiB1 positivity in 10% of the neoplastic cells, and 40% necrotic areas (arrow). (B) In contrast, the percentage of MiB1 positive neoplastic cells was increased to 80% in a tumor obtained after transplantation of del 8 E-cadherin expressing cells. The extent of necrotic areas in this tumor was 60% (arrow). (C-G) Higher magnification demonstrates differences in MiB1 positivity: (C) Parental MDA-MB-435S cells (40% positivity), (D) wt E-cadherin expressing cells (10% positivity), (E) del 8 E-cadherin expressing cells (80% positivity), (F) del 9 E-cadherin expressing cells (45% positivity), (G) p8 E-cadherin expressing cells (50% positivity). Original magnification: (A, B)×50; (C-G)×200.

FIG. 20: Sequence analysis of the E-cadherin gene
Genomic DNA was isolated MDA-MB-435S cells transfected with wt- or p8-EcadEGFP E-cadherin and the E-cadherin gene was sequenced. The mutations introduced into the stop codon (TAGc [symbol] TTAG) and the p8 point mutation in exon 8 are indicated (GAT [symbol] GCT, D370A).

FIG. 21: Detection of EGFP and E-cadherin expression in MDA-MB-435 transfectants by Western blot analysis
Parental MDA-MB-435 cells, pEGFP-N2 vector transfectants as well as wt-EcadEGFP and p8-EcadEGFP expressing cells were cultivated for two days, lysed and separated by SDS/PAGE. Primary antibody dilutions were: anti-E-cadherin antibody 1:2000, anti-GFP antibody 1:1000. The detection was performed with ECL. A: Western blot analysis with anti-E-cadherin antibody. B: Western blot analysis with monoclonal anti-GFP-antibody. A431 cells were used as control. FP: Fusion protein. wt: wt-E-Cadherin.

FIG. 22: FACS analysis to demonstrate EcadEGFP expression
Parental, EGFP, wt-EcadEGFP or p8-EcadEGFP expessing MDA-MB-435S cells were cultivate for 3 days on non-coated dishes and subsequently subjected to FACS analysis.

FIG. 23: Staining of the actin cytoskeleton with rhodamin-coupled phalliodin wt-EcadEGFP (A-C) or p8EcadEGFP (D-E) expressing MDA-MB-435S cells were seeded on glass plates and fixed with formaldehyd after 3 days. The actin cytokeleton was stained with rhodamine-coupled phalloidin. Optical slices at 0.1 μm intervalls were obtained by laser scanning microscopy. One representative of 5 slices (0,5 μm) per clone is shown. A,D: EcadEGFP-fluorescence. B,E: Actin staining. C,F: Merge of EcadEGFP and actin staining. Yellow regions indicate co-localization of EcadEGFP (green) and the actin cytoskeleton (red). Bars represent 20 μm.

FIG. 24: Detection of β-catenin localization by immunofluoresence
MDA-MB-435S cells transfected with pEGFP (A-C), wt-EcadEGFP (D-F) or p8-EcadEGFP (G-1), were seeded on glass plates and fixed with formaldehyd after 3 days. β-catenin was detected with polyclonal anti-α-catenin antibody and a TRITC-conjugated anti-β-catenin antibody. A,D,G: EGFP/EcadEGFP-fluorescence. B,E,H: β-catenin staining, C,F,I: Merge of EcadEGFP or EGFP and β-catenin. Yellow regions show co-localization of EcadEGFP or EGFP (green) and α-catenin (red). Bars represent 20 μm.

FIG. 25: Localization of wt-EcadEGFP
A: E-cadherin structures between neighbouring cells, forming zipper-like structures which are marked by arrows. B: Enhanced E-cadherin accumulation at cell cell contact sites, marked by an arrow. Bars in A) and B) represent 10 μm.

FIG. 26: p8EcadEGFP expressing MDA-MB-435S cells
1×10⁵ p8-EcadEGFP expressing MDA-MB-435S cells were seeded on collagen I coated dishes with glass bottom. Cell migration and p8EcadEGFP localization were observed for a time period of 9 h. Shown are 16 photographs, taken in 3 min-intervalls within 45 min. Arrows point to lamellipodiae with enhanced p8EcadEGFP, to filopodiae and to transient cell cell contacts. The bar representents 20 μm.

FIG. 27: Cell paths of wt- and p8-EcadEGFP expressing MDA-MB-435S cells
Paths of 10 randomly chosen cells were analysed semi-automatically within 4 h, using the Zeiss Laser Scanning Microscope Software. The bar represents 10 μm. Concentrations: 6,3 μM, 100 ng/ml EGF.

FIG. 28: Cell paths of wt-and p8-EcadEGFP expressing MDA-MB-435S cells
Paths of 10 randomly chosen cells are shown. The median is shown in red. Concentrations: 6,3 μM, 100 ng/ml EGF.

FIG. 29: Localization of wt-E-CadEGFP
1×10⁵ wt-EcadEGFP expressing MDA-MB-435S cells were seeded on Collagen I coated dishes with glass bottom. Cells were traced for 192 min, pictures were taken at 24 min-intervals. The arrow points to E-cadherin plaques. The bar represents 20 μm.

FIG. 30: Influence of Tyrphostin AG1478 on E-cadherin localization in wt-EcadEGFP expressing cells
1×10⁵ wt-EcadEGFP expressing MDA-MB-435S cells were seeded on Collagen I coated dishes with glass bottom. 6,3 μM Tyrphostin was added to the cells immediately before start. Cells were traced for 192 min, pictures were taken at 24 min-intervals. The arrow points to E-cadherin plaques. The bar represents 20 μm.

FIG. 31: Influence of EGF on the localization of E-cadherin in wt-EcadEGFP expressing MDA-MB-435S cells
1×10⁵ wt-EcadEGFP expressing MDA-MB-435S cells were seeded on Collagen I coated dishes with glass bottom and serum starved overnight. EGF (100 ng/ml) was added to the cells immediately before start. Cells were traced for 192 min, pictures were taken at 24 min-intervals. The arrow points to E-cadherin accumulations in lamellopodiae and traniently formed cell cell contacts. The bar represents 20 μm.

FIG. 32: p8-E-CadEGFP expressing MDA-MB-435S cells
1×10⁵ p8-EcadEGFP expressing MDA-MB-435S cells were seeded on Collagen I coated dishes with glass bottom. Cells were traced for 192 min, pictures were taken at 24 min-intervals. The arrow points to E-cadherin accumulations in lamellopodiae and transiently formed cell cell contacts. The bar represents 20 μm.

FIG. 33: Influence of Tyrphostin AG1478 on E-cadherin localization in p8 EcadEGFP expressing cells
1×10⁵ p8-EcadEGFP expressing MDA-MB-435S cells were seeded on Collagen I coated dishes with glass bottom. 6,3 μM Tyrphostin was added to the cells immediately before start. Cells were traced for 192 min, pictures were taken at 24 min-intervals. The arrow points to E-cadherin enhanced cell cell contacts. The bar represents 20 μm.

FIG. 34: Influence of EGF on the localization of E-cadherin in p8-EcadEGFP expressing MDA-MB-435S cells
1×10⁵ p8-EcadEGFP expressing MDA-MB-435S cells were seeded on Collagen I coated dishes with glass bottom and serum starved overnight. EGF (100 ng/ml) was added to the cells immediately before start. Cells were traced for 192 min, pictures were taken at 24 min-intervals. The arrow points to E-cadherin accumulations in lamellopodiae. The bar represents 20 μm.

FIG. 35: Immunohistochemical detection of EGFR in gastric carcinoma
Examples of different gastric adenocarcinomas displaying variable degrees of intensity of EGFR staining at the invasion front. A: Adenocarcinoma of mixed type with 3+intensity of EGFR staining; the arrow indicates intralymphatic carcinoma. B: Adenocarcinoma of intestinal type, example of heterogeneous staining with 0 to 3+ intensity. C: Adenocarcinoma of diffuse type with 2+ intensity of membrane staining. D: Adenocarcinoma of diffuse type with 1+intensity of membrane staining. Original magnification: ×100.

FIG. 36: Survival impact of EGFR score in gastric adenocarcinoma
The log rank test statistical analysis indicates a global log rank p=0.0083 when survival impact of EGFR score was evaluated. EGFR score 0/1+ were significantly associated with increased survival when compared to 2+/3+ (p=0.0006). s: significant; ns: not significant.

FIG. 37: Influence of the percentage of EGFR reactive neoplastic cells on survival
The log rank test statistical analysis indicates a p=0.0688 when the impact of the percentage of EGFR reactive neoplastic cells on survival was determined. ns: not significant.

FIG. 38: Influence of the presence of EGFR reactive cells infiltrating muscle layer and subserosa on survival
The log rank test statistical analysis indicates a p=0.0004 when the impact of EGFR reactive cells infiltrating muscle layer or subserosa on survival was investigated. s: significant.

FIG. 39: Influence of the residual disease status on survival
The log rank test statistical analysis indicates a p=0.0028 when the impact of the residual disease status on survival was investigated. s: significant.

FIG. 40: Survival impact of del 8 or del 9 E-cadherin reactivty in patients with stage I/Il or III/IV in diffuse and mixed type gastric adenocarcinoma using Kaplan Meier method
The log rank test statistical analysis indicates a global log rank p=0.009 when survival impact of del 8 or del 9 E-cadherin reactivty in patients with stage I/II or III/IV was evaluated.

FIG. 41: Survival impact of EGFR score of reactivity in patients with stage I/II or III/IV in diffuse and mixed type gastric adenocarcinoma using Kaplan Meier method
The log rank test statistical analysis indicates a global log rank p=0.0326 when survival impact of del 8 or del 9 E-cadherin reactivty in patients with stage I/II or III/IV was evaluated.

The examples illustrate the invention.

### EXAMPLE I

### Materials and Methods Employed in the Following Illustrative Examples I TO IV

a) Abbreviations Employed Herein

PKB, Akt/protein kinase B; del 8 E-cadherin, E-cadherin with deletion of exon 8; del 9 E-cadherin, E-cadherin with deletion of exon 9; DMEM, Dulbecco's modified Eagle medium; ECM, extracellular matrix; EGF, epidermal growth factor; EGFR, epidermal growth factor receptor; FCS, fetal calf serum; p8 E-cadherin, E-cadherin with point mutation in exon 8; PBS, phosphate-buffered saline; PI 3-kinase, phosphatidylinositol 3-kinase; PI3P, phosphatidylinositol 3-phosphate; PMSF, phenylmethyl sulfonylfluoride; TLC, thin layer chromatography; wt E-cadherin, wild-type E-cadherin; DHPLC: denaturing high pressure liquid chromatography; DMEM, Dulbecco's modified Eagle medium; 5-FU, 5-fluorouracil; h: hours; p8 E-cadherin, E-cadherin with point mutation in exon 8; PCR: polymerase chain reaction.

b1) Cell Cultivation and Transfection

The human E-cadherin-negative mammary carcinoma cell line MDA-MB-435S (ATCC, Rockeville, USA) and the E-cadherin-cDNA transfected derivatives that were described by Handschuh (Oncogene 18 (1999), 44301-4312) were grown in Dulbecco's modified Eagle medium (DMEM, Life Technologies, Eggenstein, Germany) supplemented with 10% fetal calf serum (FCS, PAN Biotech, Aidenbach, Germany) and penicillin-streptomycin (50 IU/ml and 50 μg/ml; Life Technologies, Eggenstein, Germany) at 37° C. and 5% CO₂.

b2) Cell Motility Studies

For time-lapse laser scanning microscopy, cells were cultivated in a microscope-coupled incubation chamber (Zeiss, Jena, Germany) at 37° C. under 5% CO₂. Cells were seeded at a density of 2×10⁵ cells per 3.5-cm in plates with a glass bottom which were purchased either uncoated or coated with poly-lysine from MatTek Corporation (Ashland, Mass., USA). Uncoated plates were coated for 4 h at 37° C. with collagen I (100 μg/ml, Sigma, Deisenhofen, Germany) or overnight at 4° C. with fibronectin (10 μg/ml, Sigma) or vitronectin (10 μg/ml, Becton Dickinson, Bedford, USA). Kinase inhibitors were used at final concentrations of 50 μM (PD 98059, Sigma, Deisenhofen, Germany), 40 μM (LY 294002, Calbiochem, Schwalbach, Germany), or 6.3 μM (Tyrphostin AG 1478, Sigma). EGF was used at a concentration of 100 ng/ml (Sigma).

Phase contrast images were taken at 3 min intervals with an Axiovert laser scanning microscope LSM 510 (Zeiss) with lens PNF 20×/0.4 PH2 and a helium-neon laser at 543 nm in transmission scanning mode. The percentage of motile cells was measured by drawing the outlines of cells on the screen and counting the cells which moved completely out of the initial area within the recording time of 7 h. Semiautomatic tracing of cell nuclei using the laser scanning microscope software from Zeiss allowed determination of the individual cell speed. The calculation of the cell speed is based on the division of the displacement of an individual cell divided by the total time of recording.

b3) Cell Adhesion Assay

Flat-bottom 96-well microtiter plates (Nunc, Wiesbaden-Biebrich, Germany) were coated overnight at 4° C. with poly-L-lysine (150 μg/cm²), collagen I (10 μg/cm²), fibronectin (0.9 μg/cm²), or vitronectin (0.45 μg/cm²). Poly-L-lysine, collagen I, fibronectin and vitronectin were purchased from Sigma. Cells were treated with versene (0.53 mM EDTA in phosphate-buffered saline (PBS), Life Technologies) in order to preserve cell surface receptors and then seeded on the different matrices at a density of 10⁴ cells per well in 100 μl DMEM without FCS. Cells were allowed to adhere to the substrata for 20 min at 37° C. and 5% CO₂ in a cell culture incubator. Unattached cells were removed by washing two times with Dulbecco's PBS without calcium and magnesium (PAA Laboratories, Cölbe, Germany). Fresh medium was added to the residual attached cells and cell viability was determined by XTT-cell proliferation and viability assay (Roche Molecular Biochemicals, Mannheim, Germany). 50 μl XTT labeling mixture was added to each well. The cleavage of the tetrazolium salt XTT to form a formazan dye that occurs in metabolically active viable cells was quantified spectrophotometrically by measuring the absorbance of the formazan product at 450 nm by an ELISA plate reader. The absorbance values obtained when culture medium without cells was assayed was subtracted from the values obtained with cells. Quadruplicate determinations were performed for each value.

b4) Flow Cytometry

Cells were harvested with versene and 5×10⁵ cells were incubated with 4 μg/ml monoclonal antibodies directed to α1 or β1 integrin (Chemicon, Temecula, USA) for 1 h on ice in PBS, washed with 0.1% sodium azide and 0.1% bovine serum albumine (Sigma) and stained with DTAF-conjugated anti mouse IgG (Jackson ImmunoResearch Laboratories, West Grove, USA) for 1 h on ice. Purified mouse IgG2a and IgG1 (Pharmingen, Heidelberg, Germany) were used as κ immunoglobulin isotype controls. Cells were analyzed on a Beckman Coulter Epics XL (Beckman Coulter, Krefeld, Germanyl .

b5) Western Blot

For immunoblot analysis, cells were seeded at a density of 6×10⁵ cells per 10 cm tissue culture dish and lysed 5 h later with 500 μL-CAM buffer (140 mM NaCl, 4.7 mM KCl, 0.7 mM MgSO₄, 1.2 mM CaCl₂, 10 mM Hepes pH 7.4, containing 1% (v/v) Triton-X-100 and 1 mM phenylmethylsulfonylfluoride (PMSF) (Cunningham, Proc. Acad. Sci. USA 81 (1984), 5787-5791). Proteins were separated by 7% SDS-polyacrylamide gel electrophoresis followed by transfer to nitrocellulose (Schleicher & Schuell, Dassel, Germany) or PVDF (Bio-Rad Laboratories, München, Germany) membranes. Polyclonal antibodies against total or activated kinases were purchased from New England Biolabs (Frankfurt, Germany): Akt antibody (#9272), detecting total Akt kinase levels, phospho-Akt antibody (Ser473, #9270), detecting phosphorylated Ser 473 in Akt1, Akt2 and Akt3, MAP kinase antibody (# 9102), detecting total MAP kinase, phospho-p44/42 MAP kinase (#9101) detecting activated MAP kinase phosphorylated at Thr 202/Thr 204. Monoclonal antibody against p53 was purchased from Oncogene Research Products (Ab-6, #OP43). Polyclonal antibodies against total or activated p38 MAP kinases were purchased from New England Biolabs. Phospho-p38 MAP kinase polyclonal antibody (#9211) detects phosphorylated threonine 180 and tyrosine 182 p38 MAP kinase (#9212). For signal detection the enhanced chemoluminescence system (Amersham Pharmacia Biotech, Braunschweig, Germany) was used. Densitometric analysis was performed with Scion Image Software from Scion Corporation (Frederick, USA).

b6) PI 3-Kinase Assay

For the detection of PI 3-kinase activity, a protocol from Upstate Biotechnology (Lake Placid, USA) was used. Cells were seeded at a density of 1.6×10⁶ cells per 15 cm tissue culture plate and grown for 5 h in DMEM supplemented with 10% FCS. As a positive control, cells were treated with 100 ng/ml EGF for 2 min prior to lysis. Cells were lysed with 137 mM NaCl, 20 mM Tris-HCl/pH 7.4, 1 mM CaCl₂, 1 mM MgCl₂, and 0.1 mM sodium orthovanadate, 1% NP-40 and 1 mM PMSF. After preclearing with protein A agarose beads (Amersham Pharmacia Biotech), anti-PI 3-kinase p85 antibody (#06-195, Upstate Biotechnology) was added to the lysates for 2 h at 4° C. and the immunoprecipitates were collected by centrifugation. Each pellet was washed three times with lysis buffer, three times with 0.1 M Tris-HCl/pH 7.4, 5 mM LiCl, 0.1 mM sodium orthovanadate, three times with TNE buffer (0.1 M Tris-HCl/pH 7.4, 150 mM NaCl, 5 mM EDTA, 0.1 mM sodium orthovanadate) and resuspended-in 50 μl TNE buffer. After addition of 20 μg phosphatidylinositol (Sigma) and 10 μl 100 mM MgCl₂, the reactions were started by adding 5 μl per sample of [gamma³³P] ATP solution (Amersham Pharmacia Biotech, 0.88 mM ATP containing 20 μCi [gamma³³P] ATP, 2000 Ci per mmole and 20 mM MgCl₂). As a control, 10 μM LY 294002 were included in the reaction sample. After incubation for 10 min at 370C, the reaction was stopped with 6 N HCl. Chloroform/methanol (1:1, v/v), was added to extract the radiolabeled lipid. The organic phase was spoted on a thin layer chromatography (TLC) plate (Merck, Darmstadt, Germany) and the TLC plate was developed in chloroform/methanol/H₂O/NH₄OH (43:38:7:5). Radiolabeled lipids were visualized by autoradiography.

### EXAMPLE II

### Expression of Mutant E-Cadherin Enhances Random Cell Movement as Compared to Wt E-Cadherin

In the present study, MDA-MB-435S mammary carcinoma cell transfectants expressing either wt or mutant E-cadherin cloned from diffuse-type gastric carcinomas were compared with respect to their individual motile behaviour. As documented in FIG. 1A, the mutations were deletions of exons 8 (del 8) or 9 (del 9) and a point mutation in exon 8 (p8) as described elsewhere in detail (Handschuh (1999) loc. cit.). Expression of wt or mutant E-cadherin-cDNA after transfection of E-cadherin-negative MDA-MB-435S cells was recently shown by Western blot analysis and immunofluorescence staining (Handschuh (1999), loc. cit.). Enhanced migration of MDA-MB-435S cells expressing mutant E-cadherin as compared to wt E-cadherin was demonstrated by wound healing assay (Handschuh (1999), loc. cit.). In the wound healing assay, the observed cell migration results from a number of combined effects, such as cell-cell and cell-matrix interactions, cell proliferation and secretion of growth factors as well as extracellular matrix (ECM) components. Therefore, in the present study, the motility of individual E-cadherin expressing cells was analyzed by time-lapse laser scanning microscopy. At least two independent cell clones were investigated for each E-cadherin expression construct used in this study.

Despite the fact that the mutant E-cadherins investigated in this study were cloned from diffuse-type gastric carcinomas, MDA-MB-435S mammary carcinoma cells were used instead of gastric carcinoma cells as recipient cells. MDA-MB-435S cells lack endogeneous E-cadherin. Methylation-associated silencing of E-cadherin gene expression (Graff, Cancer Res. 55 (1995), 5195-5199) and downregulation of E-cadherin gene expression by snail have been suggested (Cano, Nat. Cell Biol. 2 (2000), 76-83). MDA-MB-435S cells have therefore been used for studying E-cadherin function after transfection with E-cadherin expression constructs by us and other groups (Handschuh (1999), loc. cit.; Luber, Cell. Adhes. Commun. 7 (2000), 391-408; Frixen, J. Cell Biol. 113 (1991), 173-185; Meiners, Oncogene 16 (1998), 9-20). Mutations in the E-cadherin gene have been identified in diffuse-type gastric carcinomas and lobular breast cancers as well as in gynecolocial tumors (Becker, Hum. Mol. Genet. 2 (1993), 803-804; Becker, Cancer Res. 54 (1994), 3845-3852; Berx, Hum. Mutat. 12 (1998), 226-237). A mutational analysis of E-cadherin in human breast cancer cell lines revealed a deletion of exon 9 in cell line MPE600 (Hiraguri, Cancer Res. 58 (1998), 1972-1977; Van de Wetering, Cancer Res. 61 (2001), 278-284) which indicates that investigation of E-cadherin mutations in breast cancer cells is of physiological relevance.

Cells were brought in suspension by disruption of cell-cell and cell-matrix interactions and seeded on glass plates precoated with collagen type 1. Cell motility was analyzed by long-term recordings of a duration of 7 h starting 2 h after plating using a laser scanning microscope coupled with a temperature- and CO₂-controlled incubation chamber. Motile cells were defined as cells which were able to move out of their initial space within 7 h according to a method described by Marks (J. Cell Bioll. 112 (1991), 149-158). The percentage of motile cells was similar in non-transfected (30%) or wt E-cadherin expressing MDA-MB-435S cells (26%) but elevated upon expression of mutant E-cadherin: del 9 (52%), del 8 (58%) and p8 (45%, FIG. 1B).

It has been shown for a number of cell lines that the motile behavior of individual cells is variable and therefore it is advisable to investigate a large number of cells to obtain a reliable determination of cellular velocity (Hartmann-Petersen, Cytometry 40 (2000), 260-270). In order to determine the motility of individual cells, the center of the nucleus was semiautomatically traced and the speed of 60 cells per cell line derived from at least three independent experiments was calculated. The range between the minimal and the maximal cell speed and the median of cell speeds were used as motility parameters. Expression of mutant E-cadherin induced broad speed ranges and higher maximal speeds in MDA-MB-435S cells expressing del 9 (3.0-58.1 μm/h), del 8 (4.6-53.0 μm/h) or p8 E-cadherin (3.6-59.5 μm/h) compared to wt E-cadherin (1.2-20.8 μm/h) expressing or parental (3.4-46.6 μm/h) cells (FIG. 1C). The medians of cell speeds were higher in cells expressing del 9 (10.4 μm/h), del 8 (16.0 μm/h) or p8 E-cadherin (10.7 μm/h) or non-transfected parental cells (10.4 μm/h) as compared to wt E-cadherin (7.0 μm/h) expressing cells (FIG. 1C). These data suggest that expression of wt E-cadherin, in contrast to mutant E-cadherin, considerably decreases the median of cell speeds of a cell population, presumably by increasing cell-to-cell contacts which counteract cell migration.

Diffuse-type gastric carcinomas and lobular breast cancers invade surrounding tissues as single cells (Birchmeier, Biochim. Biophys. Acta 1198 (1994), 11-26; Hirohashi, Am. J. Pathol. 153 (1998), 333-339; Oka, Cancer Res. 53 (1993), 1696-1701). The question whether E-cadherin mutations cloned from diffuse-type gastric carcinomas actively increase cell migration in a wound healing assay or whether they only abolish E-cadherin function was previously addressed and it was found that both is true: in highly motile L929 fibroblasts, wt E-cadherin caused a dramatic reduction of cell migration into the wound, whereas mutant E-cadherin resulted only in partial reduction of cell migration (Handschuh (1999), loc. cit.). These results indicate a partial loss of the migration-inhibiting function of E-cadherin by mutations in the extracellular domain. On the other hand, parental or wt E-cadherin expressing MDA-MB-435S cells did hardly enter the wound, whereas 10 times more cells expressing del 9 E-cadherin, and 100 times more cells expressing del 8 or p8 E-cadherin moved into the wound. These results provided evidence that mutant E-cadherins result in a gain of function and actively enhance cell migration (Handschuh (1999), loc. cit.).

In the present study, parental and wt E-cadherin expressing MDA-MB-435S cells showed a low percentage of motile cells (FIG. 1B, 30% or 26%, respectively), whereas cell motility was increased by expression of mutant E-cadherin (del 9: 52%, del & 58%, p8: 45%). These data are in accordance with the wound healing assays. Despite of the expression of N-cadherin on the RNA and protein level (Handschuh, J. Mol. Biol. 314 (2001), 455-464), parental MDA-MB-435S cells were not highly motile in the experiments described herein. The fact that the median of cell speeds of untransfected MDA-MB-435S cells was within the same range as for mutant E-cadherin expressing cells (FIG. 1C), is on the first glance contradictory. However, time-lapse laser scanning microscopy showed that parental MDA-MB-435S cells moved within a clone or rotated around their own axis, inspite of actively leaving a clone like mutant E-cadherin expressing cells.

In the following experiments, del 8 E-cadherin, as a prototype of mutant E-cadherin, was compared to wt E-cadherin. The motile behaviour of cells in the absence of a chemotactic agent was described as random movement (Dunn, Agents Actions Suppl. 12 (1983), 14-33). As shown in FIG. 2A, MDA-MB-435S cells expressing wt E-cadherin formed small colonies even at low density and revealed low locomotion activity. In contrast, cells expressing del 8 E-cadherin had a strong tendency to separate from each other and to form lamellipodial protrusions (FIG. 2B). A significant number of cells underwent random cell migration which was characterized by frequent and abrupt changes of the direction of movement. Often, del 8 E-cadherin expressing cells separated from the surrounding cells immediately after cell division rather than attaching to other cells and forming colonies. Plots derived from the paths of 10 randomly chosen cells show that mutant E-cadherin affects the style of cell movement and enhances random cell migration (FIG. 3).

### EXAMPLE III

### Enhancement of Cell Motility by Mutant E-Cadherin Is Dependent on ECM Conditions

Cell adhesion to the substrate plays a critical role in cell migration. Therefore, the influence of extracellular matrices on cell motility and adhesion was determined.

Eucaryotic cell motility plays a pivotal role in physiological and pathological processes, such as embryonic development, wound healing as well as tumor invasion and metastasis. Cell migration requires interactions between cellular adhesion molecules and the extracellular matrix at the leading edge of the cell and release of adhesive interactions at the trailing end (Lauffenburger, Cell 84 (1996), 359-369).

Cells plated on poly-lysine, fibronectin, or vitronectin were well-spread, but only cells expressing del 8 E-cadherin plated on collagen I showed the typical morphology of migrating cells with broad lamellipodiae at the leading edge and a trailing end.

In order to determine the adhesion of cells on the various ECM proteins, MDA-MB-435S cells expressing wt or del 8 E-cadherin were allowed to adhere for 20 min on cell culture plates precoated with poly-lysine, collagen I, vitronectin or fibronectin and the attached viable cells were quantified (FIG. 4A). Cell adhesion on poly-lysine and vitronectin was stronger than on collagen I or fibronectin and within the same range for wt or del 8 E-cadherin expressing cells. Next, the relationship between adhesion to the ECM and cell motility was examined (FIG. 4B). Cell motility was inversely correlated with cell-matrix adhesion. Only del 8 E-cadherin expressing cells plated on collagen I were able to migrate significantly (FIG. 4B, C). Flow cytometric analysis of collagen-specific integrin α1 which pairs with integrin β1 subunit carried out with non-transfected, wt and mutant E-cadherin expressing MDA-MB-435S cells revealed that both integrins are expressed at similar levels in all tested cell lines (FIG. 4D).

MDA-MB-435S cells expressing mutant E-cadherin showed increased cellular motility compared to wt E-cadherin expressing cells in a motility assay based on time-lapse laser scanning microscopy. As motility parameters, determination of the percentage of cells which leave the initial space within the observation time of 7 h as well as calculation of cellular velocity were used. Cell speeds within a transfected cell line differed up to 10 fold among individual cells. This might be due to the investigation of asynchronous cell populations because cells have been shown to exhibit differences in their motile behaviour according to their cell cycle phases in a different study (Hartmann-Petersen (2000), loc. cit.). Maximal cell speeds in mutant E-cadherin-cDNA expressing MDA-MB-435S cells was observed, suggesting that reduced cell-cell adhesive interactions are critical for cellular motility in vitro and presumably also in vivo.

A mathematical relationship between cell-substratum adhesion and cell migration was defined by Palecek (Nature 385 (1997), 537-540). Accordingly, the extent of cell adhesion to the ECM depends on the concentration of cell surface integrins and ECM proteins. Low integrin or ECM protein concentration resulted in weakly attached cells with low tractive forces. Increasing attachment led to an optimal rate of cell migration. With further attachment, however, cells displayed impaired motility presumably due to the inability to cycle between the adherent and nonadherent state. In this study, the extent of cell adhesion to different ECM proteins was inversely correlated with cell motility, suggesting that ECM proteins influence mutant E-cadherin-enhanced cell motility. Collagen I resulted in highest cell motilities of mutant E-cadherin expressing MDA-MB-435S cells, presumably because this ECM protein resulted in sufficient attachment and detachment of cells to enable cell migration. MDA-MB-435S cells were found to express α1 and β1 integrin which mediate as a heterodimer binding of collagen I. Mutations in E-cadherin did not influence the α1 and β3 integrin expression patterns of MDA-MB-435S transfectants, ruling out the possibility that mutant E-cadherin transcriptionally regulates the collagen I receptor expression pattern. Moreover, E-cadherin mutations did not alter the α2, α3 and αv integrin expression patterns (to be published elsewhere). Also, in this study E-cadherin expression was not transcriptionally downregulated by collagen I as observed by other authors [43], presumably because E-cadherin expression was driven by the β-actin promoter in our constructs and not by the native E-cadherin promoter.

Taken together, these data suggest that ECM conditions influence the migratory behaviour of cells and that strong cell-substrate adhesive interactions counteract cell migration. wt E-cadherin expressing MDA-MB-435S cells show low locomotion activity on all ECM proteins tested, indicating that also strong cell-cell adhesive forces block cell motility.

### EXAMPLE IV

### Motility Enhancement by Mutant E-Cadherin is Sensitive to Inhibitors Of EGFR and PI 3-Kinase

Different members of the cadherin family have multiple functions. E-cadherin is implicated in the maintenance of an epithelial and non-invasive phenotype (Frixen (1991), loc. cit.) and actively induces mesenchymal to epithelial transition (Auersperg, Proc. Natl. Acad. Sci. USA 96 (1999), 6249-6254). In contrast to N-cadherin and cadherin-11 which are known to upregulate cell motility, E-cadherin is known to counteract cell motility and invasion (Frixen (1999), loc. cit.). However, previous work revealed that E-cadherin, mutated within the linker region between extracellular domain 2 and 3, results in an increase in cell migration (Handschuh (1999), loc. cit.).

Cell migration is influenced by a number of factors including cell-cell and cell-matrix interactions as well as transduction of extracellular signals into cells. Next, the contribution of the cell motility-associated EGFR pathway to mutant E-cadherin-enhanced cell motility was investigated. The percentage of motile del 8 E-cadherin-expressing MDA-MB-435S cells as well as the median and range of cell speeds decreased to the level of wt E-cadherin expressing cells upon treatment with the specific EGFR kinase inhibitor Tyrphostin AG 1478 (FIG. 5A, B) (Osherov, Eur. J. Biochem. 225 (1994), 1047-1053).

EGFR activates diverse downstream signaling molecules including PI 3-kinase, Akt/PKB and MAP kinase (Prenzel, Endocr. Relat. Cancer 8 (2001), 11-31). In order to determine the importance of these signaling molecules for mutant E-cadherin-enhanced cell motility, cell tracking experiments were performed in the presence of MAP kinase kinase inhibitor PD 98059 which prevents threonine and tyrosine phosphorylation of MAP kinase (Alessi, J. Biol. Chem. 270 (1995), 27489-27494; Dudley, Proc. Natl. Acad. Sci. USA 92 (1995), 7686-7689), or the synthetic PI-3 kinase inhibitor LY 294002 (Baumann, Proc. Natl. Acad. Sci. USA 95 (1998), 14066-14070). Cell motility could be blocked by LY 294002, whereas PD 98059 was less effective (FIG. 5A, B). In order to determine whether PI 3-kinase was activated by mutant E-cadherin, PI 3-kinase activity was measured. Similar activation levels were observed for all tested cell lines, suggesting that a basic activation level rather than stimulation of PI 3-kinase by mutant E-cadherin is necessary for enhanced cell motility stimulated by mutant E-cadherin (FIG. 6). PI 3-kinase activation was sensitive to LY 294002 (FIG. 6).

Next, the effect of epidermal growth factor (EGF) on the motility of MDA-MB-435S transfectants was tested. The percentage of motile wt E-cadherin expressing MDA-MB-435S cells was increased to the level of del 8 E-cadherin expressing cells in response to EGF treatment (FIG. 5A). EGF-treated cells developed filopodia and lost cell to cell contacts (FIG. 5C). The influence of PD 98059, LY 294002, Tyrphostin AG 1478 and EGF on cell morphology is shown in FIG. 5C. The specifity of the kinase inhibitors used in this study was tested by Western blot analysis using phosphorylation-specific antibodies. Tyrphostin AG 1478 completely blocked EGF-induced activation of MAP and Akt/PKB kinases (data not shown). LY 294002 inhibited EGF-induced Akt/PKB activation to a similar extent and PD 98059 effectively blocked MAP kinase phosphorylation (data not shown).

In the present study, it was demonstrated that the motility promoting activity of mutant E-cadherin is sensitive to treatment with the EGFR specific inhibitor Tyrphostin AG 1478. EGFR has been shown to directly interact with the E-cadherin/catenin complex said interaction being mediated by β-catenin which is tyrosine phosphorylated in response to EGF treatment (Hoschuetzky, J. Cell Biol. 127 (1994), 1375-1380; Takahashi, Oncogene 15 (1997), 71-78). A direct interaction between the mutant E-cadherin/catenin complex and EGFR might activate cell motility by receptor crosstalk.

The motility promoting function of other members of the cadherin familiy and the involvement of cellular signalling pathways has been investigated in different studies. In contrast to E-cadherin, N-cadherin was suggested to induce an epithelial to mesenchymal transition and to promote motility, invasion, and metastasis of cancer cells (Nieman, J. Cell Biol. 147 (1999), 631-644; Hazan, J. Biol. Chem. 273 (1998), 9078-9084). The extracellular domain 4 of N-cadherin was shown to mediate the epithelial to mesenchymal transition and increased motility which indicates that the motility promoting activity of N-cadherin is distinct from the adhesive function which resides within extracellular domain 1 (Kim, J. Cell Biol. 151 (2000), 1193-1206). The motility promoting function of N-cadherin has been shown to be dramatically enhanced by fibroblast growth factor (FGF)-2 (Hazan (1998), loc. cit.). It was speculated that an interaction exists between N-cadherin and the FGF receptor which leads to increased cell motility. This idea is supported by the results of other studies which suggest that N-cadherin can interact with FGF receptors (Doherty, Mol. Cell Neurosci. 8 (1996), 99-111; Peluso, Biol. Signals Recept. 9 (2000), 115-121) and that N-cadherin-mediated cell motility of breast cancer cells can be blocked by an inhibitor of the FGF-mediated signaling pathway (Niemann (1999), loc. cit.). The relationship of another member of the cadherin family with a signaling pathway was also shown for VE-cadherin which forms a complex with β-catenin, PI 3-kinase and VEGFR-2, thereby activating Akt kinase and endothelial cell survival (Carmeliet, Cell 98 (1999), 147-157).

Stimulation of MDA-MB-435 cells with EGF has been demonstrated to activate Pi 3-kinase, as shown by rapid recruitment of the p85 subunit of PI-3 kinase to the phosphotyrosine-containing cellular fraction (Adelsman, Mol. Biol. Cell 10 (1999), 2861-2878). In this study, EGF treatment of wt E-cadherin expressing MDA-MB-435S cells enhanced cellular motility, which identifies the EGFR pathway as an important regulator of cell motility in our system. Enhancement of cell motility caused by mutant E-cadherin was inhibited by addition of the PI 3-kinase inhibitor LY 294002 Pi 3-kinase was not stronger activated in mutant E-cadherin expressing cells as compared to wt E-cadherin expressing cells, indicating that basal PI 3-kinase activity is necessary for the effect.

### EXAMPLE V

### AKT/PKB Activity is Increased by Expression of WT As Compared to Mutant E-Cadherin, whereas Members of the MAP Kinase Family are not Affected

The importance of MAP kinase activation for cell adhesion, spreading, and motility has recently been demonstrated (Klempke, J. Cell Biol. 137 (1997), 481-492; Renshaw, EMBO J. 16 (1997), 5592-5599; Fincham, EMBO J. 19 (2000), 2911-2923). To further investigate the role of p44/42 MAP kinase, a downstream signaling molecule of PI 3-kinase, in mutant E-cadherin-enhanced cell motility, p44/42 MAP kinase expression levels and activities were measured in non-transfected and wt or mutant E-cadherin-cDNA expressing MDA-MB-435S cells.

Western blot analysis performed with antibodies specific for the phosphorylated, active forms of MAP kinases p44 and p42 or detecting total MAP kinase revealed a a slight increase of p44 and p42 MAP kinase activity in MDA-MB-435S cells expressing wt or mutant E-cadherin as compared to non-transfected parental cells on uncoated or collagen I coated tissue cell culture plates. This increase was accompanied by elevated MAP kinase steady-state expression levels (FIG. 7A).

Next, activity and expression of Akt/PKB, another downstream molecule of PI 3-kinase, were investigated in non-transfected and wt or mutant E-cadherin-cDNA expressing MDA-MB-435S cells (FIG. 7B). By densitometric analysis of Western blots, it was found that Akt/PKB was around 2fold stronger activated in cells expressing wt E-cadherin in comparison to cells expressing mutant E-cadherin or non-transfected parental cells on uncoated and collagen I coated plates and the effect was not due to variations of the expression level.

Akt/PKB is among the main effectors of PI 3-kinase (Adelsman, (1999), loc. cit.). Akt/PKB has been shown to be activated by the formation of E-cadherin-mediated cell-cell junctions (Pece, J. Biol. Chem. 274 (1999), 19347-19351). By Western blots using phosphorylation-specific antibodies, Akt/PKB was found to be stronger activated in MDA-MB-435S cells by wt versus mutant E-cadherin in this study. Treatment with the PI 3-kinase inhibitor LY 294002 did not detectably interfere with the phosphorylation status of Akt/PKB of wt or mutant E-cadherin-cDNA expressing MDA-MB-435S cells, suggesting that beside PI 3-kinase other signaling molecules might be involved in the regulation of Akt/PKB activity. Akt/PKB has been demonstrated to be involved in mediating the anti-apoptotic effect of PI 3-kinase (Coffer, Biochem. J. 335 (1998), 1-13). Its role in regulation of cellular motility, however, is not defined.

MAP kinase p44/42 is among the main downstream effectors of PI-3 kinase (Coffer, (1998), loc. cit.) and was reported to regulate cell motility (Klempke, (1997), loc. cit.). Several publications indicate that MAP kinase is also activated by cell-cell and cell-matrix interactions. For example, integrin engagement activates MAP kinase (Fincham, (2000), loc. cit.; Zhu, Mol. Biol. Cell 6 (1995), 273-282) and E-cadherin signals to the MAP kinase pathway via EGFR engagement (Pece, J. Biol. Chem. 275 (2000), 41227-41233). Yet, in this study considerable differences in p44/42 MAP kinase activities and expression levels upon expression of wt or mutant E-cadherin-cDNAs could not be detected. Also no inhibitory effect of the MAP kinase inhibitor PD 98059 on cell motility of del 8 E-cadherin expressing MDA-MB-435S cells was observed, suggesting that MAP kinase plays only a minor role in mutant E-cadherin-enhanced cell motility in these cells.

In conclusion, the results presented herein suggest that E-cadherin mutations affect not only adhesive functions, but influence also the migratory behaviour of MDA-MB-435S cells. It was surprisingly found that increased cell motility stimulated by mutant E-cadherin is blocked by inhibitors of EGFR and Pi 3-kinase.

Inhibition of these signaling molecules with small molecule drugs is a promising approach in treatment of malignant tumors with E-cadherin mutations.

### EXAMPLE VI

### Effect of Wild-Type and Mutant E-Cadherin on Cell Proliferation And Responsiveness to the Chemotherapeutic Agents Cisplatin, Etoposide, and 5-Fluorouracil

In a further set of experiments the question whether E-cadherin mutations have an impact on the growth-suppressive function of E-cadherin was addressed. Genetic alterations play also a causative role in tumor formation and progression. Specific genetic alterations, like E-cadherin mutations, might also determine the patient's outcome after chemotherapeutic treatment. Alterations in the expression level and functionality of E-cadherin are frequently observed in human cancer. To answer the question whether the expression or mutation status of the E-cadherin gene influence chemosensitivity, the effect of cisplatin, etoposide and 5-FU, all of which have been in clinical use for several years, on parental, wt and mutant E-cadherin expressing MDA-MB-435S cells have been investigated.

In the following experiments, cells as described in section b1 of Example I were employed. Further material and methods comprise:

Reagents

Cisplatin (Sigma, Deisenhofen, Germany) was prepared as a 100 mM stock solution in DMSO. Etoposide (Calbiochem) was dissolved in DMSO as a 50 mM stock solution. 5-FU (Sigma) was prepared as a 100 mg/ml stock solution in DMSO.

XTT-Cell Proliferation and Viability Assay

Cells were seeded at a density of 2×1 0 cells per well in 96-well microtiter plates (Nunc, Wiesbaden-Biebrich, Germany) in 100 μl DMEM with 10% FCS per-well, and cell proliferation was investigated by XTT-cell proliferation and viability assay (Roche Molecular Biochemicals, Mannheim, Germany). After 24, 48 and 72 h, 50 μl XTT labeling mixture was added. The cleavage of the tetrazolium salt XTT to form a formazan dye that occurs in metabolically active viable cells was quantified spectrophotometrically by measuring the absorbance of the formazan product at 450 nm by an ELISA plate reader. The absorbance values obtained when culture medium without cells was assayed was subtracted from the values obtained with cells. Quadruplicate determinations were performed for each time point.

Sensitivity Profiling by XTT-Cell Proliferation and Viability Assay

The sensitivity of parental, wt and mutant E-cadherin expressing MDA-MB-435S cells to the chemotherapeutic agents cisplatin, etoposide and 5-FU was investigated by XTT-cell proliferation and viability assay as described above. Cells were seeded at a density of 2×10³ cells per well in 96-well microtiter plates (Nunc, Wiesbaden-Biebrich, Germany) in 100 μl DMEM with 10% FCS per well and exposed to each drug for 48 h at various concentrations. After 48 h, 50 μXTT labeling mixture was added and the absorbance values at 450 nm were measured spectrophotometrically by an ELISA plate reader. The absorbance values obtained when culture medium without cells was assayed, were subtracted from the values obtained with cells. Quadruplicate determinations were performed for each time point. The percentage of viable cells was determined corresponding to non-treated cells.

Colony Formation Assay

Chemosensitivity to cisplatin, etoposide and 5-FU of parental, wt and mutant E-cadherin expressing MDA-MB-435S cells was investigated by colony formation assay. Cells were seeded at a density of 2×10³ cells per 6-well and treated 3 h later for 2 h with cisplatin, etoposide or 5-FU. Colonies were fixed and stained after 7 days with Diff Quick Reagent (Dade Behring, Liederbach, Germany) and the colony number was determined with Scion Image Software from Scion Corporation (Frederick, USA). The percentage of colonies was determined corresponding to non-treated cells.

p53 Mutation Analysis

DHPLC which uses heteroduplex formation between wt and mutant DNA to detect mutations, was performed according to the method of Oefner and Underhill (33) on an automated DHPLC analysis system (Transgenomic, Omaha, Nebr.). p53 mutation analysis was performed with DNA isolated from parental and transfected MDA-MB-435S cells using a DNA preparation kit (Qiagen, Hilden, Germany). Detection of p53 mutations in exon 5-8 by DHPLC was performed as described previously (34). The primers and polymerase chain reaction (PCR) conditions for p53 sequence analysis were published by Keller et al. (34). The purification of PCR products from agarose gels was performed with a gel extraction kit (Qiagen). For Cycle Sequencing, the Ready Reaction Big Dye Terminator Cycle Sequencing kit (Applied Biosystems) and an automated sequencing system (ABI 377, Applied Biosystems) were used.

Results

In a further study, cell proliferation and sensitivity to chemotherapeutic agents of MDA-MB-435S mammary carcinoma cell transfectants expressing either wt or mutant E-cadherin molecules was compared with that of non-transfected, E-cadherin-negative parental cells. Mutant E-cadherin cDNA was cloned from diffuse-type gastric carcinomas and, as a control, wt E-cadherin cDNA was isolated from non-tumorous gastric mucosa as described previously (Handschuh (1999), loc. cit.). The mutations were in frame deletions of exons 8 (del 8) or 9 (del 9) and a point mutation in exon 8 (p8, D370A). Expression of wt or mutant E-cadherin in MDA-MB-435S cell transfectants was demonstrated by immunoblot and immunofluorescence analysis (Handschuh (1999), loc. cit.). At least two independent cell clones were investigated for each E-cadherin expression construct.

E-Cadherin Mutations Affect the Growth-Suppressive Function of Wt E-Cadherin

In accordance with previous observations that E-cadherin acts as a suppressor of cell growth (Watabe, J. Cell Biol. 127 (1994), 247-256; St. Croix, J. Cell Biol. 142 (1998), 557-571), we found that the proliferation rate of MDA-MB-435S cells was reduced by expression of wt E-cadherin as compared to parental cells, as shown by XTT cell proliferation and viability assay (FIG. 8). In contrast, E-cadherin mutations apparently interfere with the growth-suppressive function of E-cadherin (FIG. 8).

E-Cadherin Mutations Alter the Sensitivity to Cisplatin, While Responsiveness to Etoposide and 5-FU is not Affected

To investigate whether wt or mutant E-cadherin molecules influence the cell death response of MDA-MB-435S cells, chemosensitivity profiles of parental, as well as wt or mutant E-cadherin expressing cells to cisplatin, etoposide and 5-FU were compared. Exposure to cisplatin resulted in reduced sensitivity of wt or mutant E-cadherin expressing MDA-MB-435S cells as compared to parental cells in a XTT cell proliferation and a colony formation assay (FIG. 9). The sensitivity profiles of parental, wt and mutant E-cadherin expressing MDA-MB-435S cells in response to etoposide or 5-FU treatment were similar for all cell lines, as shown by XTT and colony formation assay (FIGS. 10 and 11).

p53 Mutation Analysis of Parental, Wt and Mutant E-Cadherin Expressing MDA-MB-435S Cells

Next, we investigated whether the observed differences in cisplatin sensitivity were caused by differences in the p53 genetic background or expression level.

Since stable transfection of cells leads to random integration of transfected cDNA into the host chromosome and can cause genetic alterations at the integration site, the mutational hot spot region exon 5-8 of p53 was analysed for mutations in DNA samples from MDA-MB-435S transfectants by denaturing high performance liquid chromatography (DHPLC) using a previously established protocol (Keller, Lab Invest. 81 (2001), 1735-1737). A change of p53 codon 266 (G266E, GGA>GAA, exon 8, nucleotide 797) was previously described in MDA-MB-435 cells (O'Connor, Cancer Res. 57 (1997), 4285-4300) and detected also in the subline MDA-MB-435S by sequence analysis (Keller (2001), loc. cit.). The same mutation pattern was found in parental MDA-MB-435S cells, as well as in wt and mutant E-cadherin expressing transfectants (FIG. 12A), indicating that transfection with E-cadherin cDNA did not alter the p53 mutation status. The presence of a wt as well as a mutant p53 allele indicate p53 heterozygosity (FIG. 12 B).

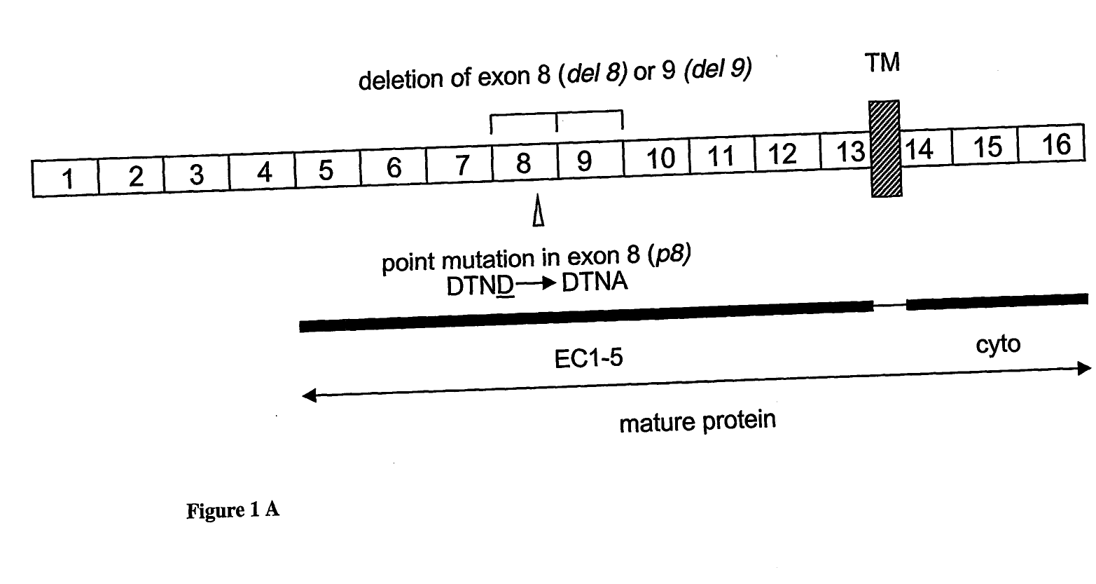

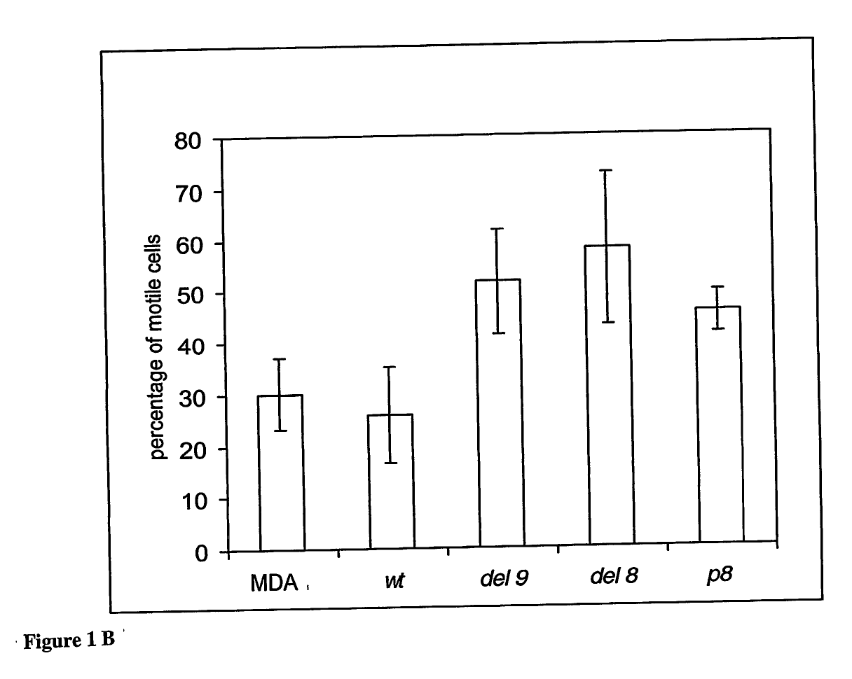

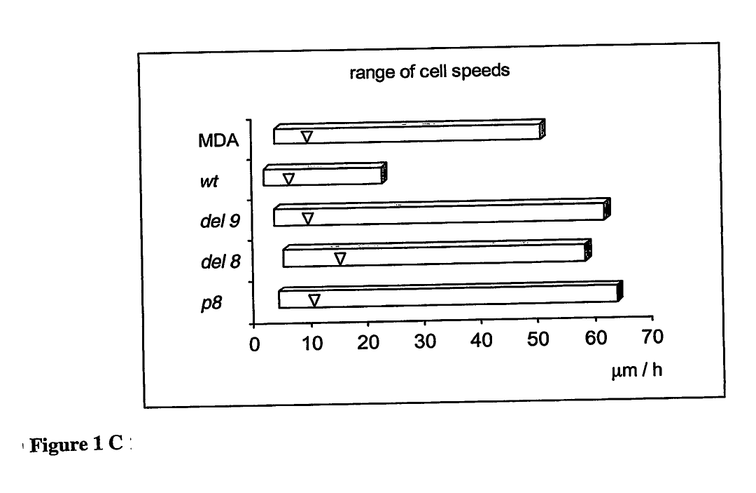

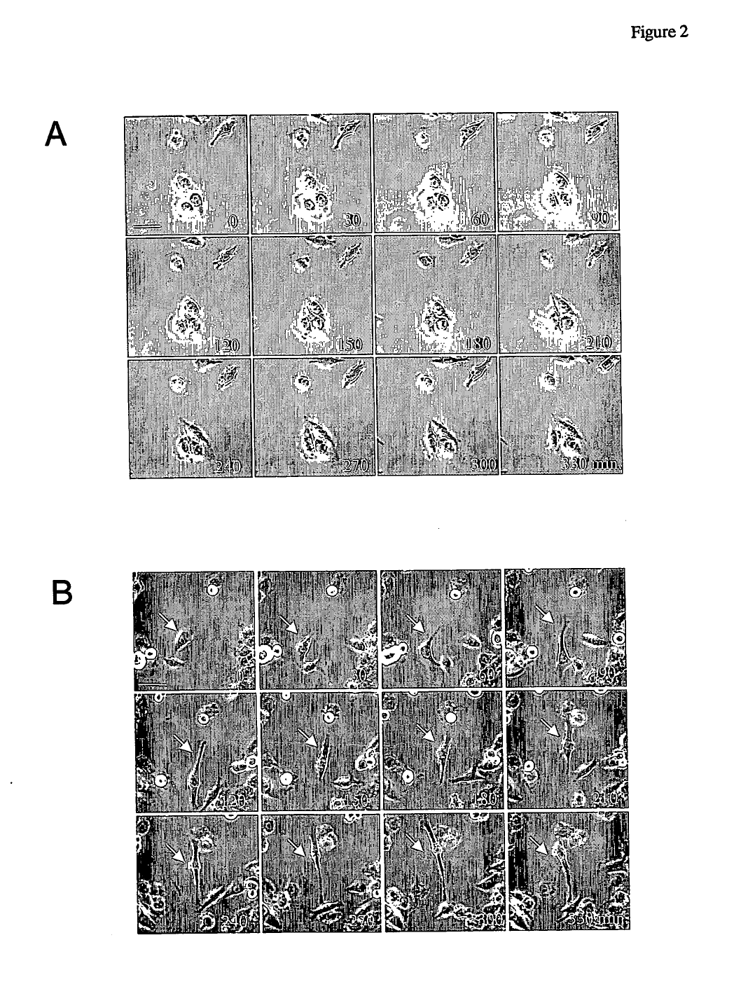

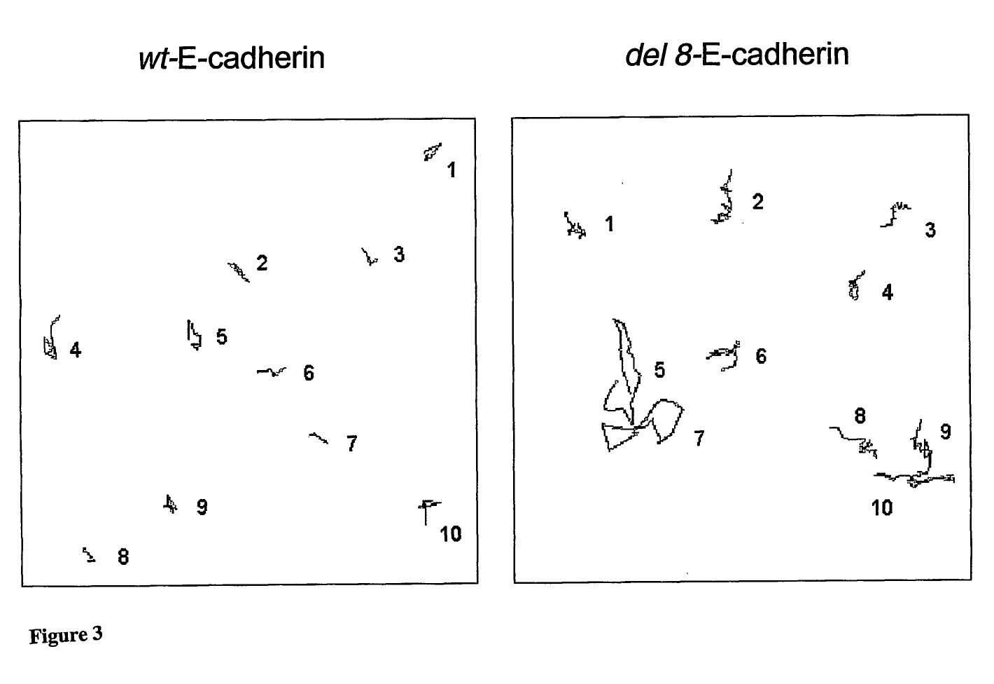

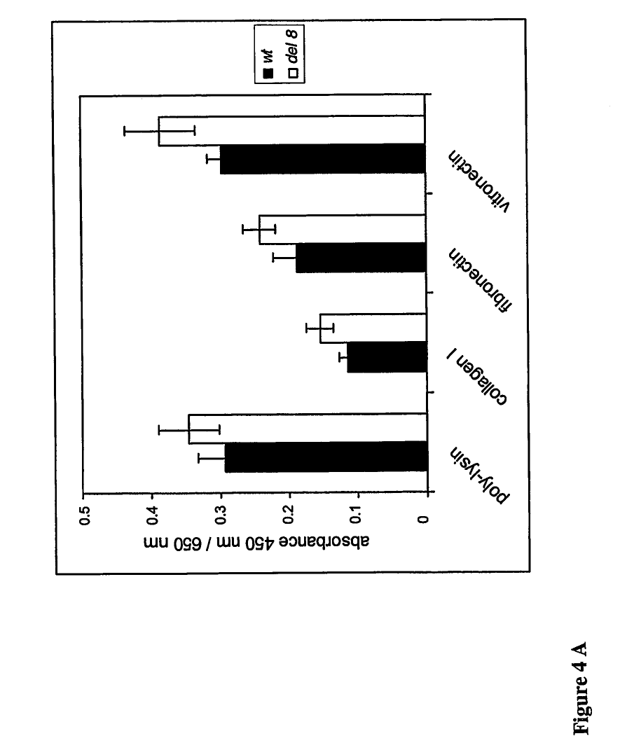

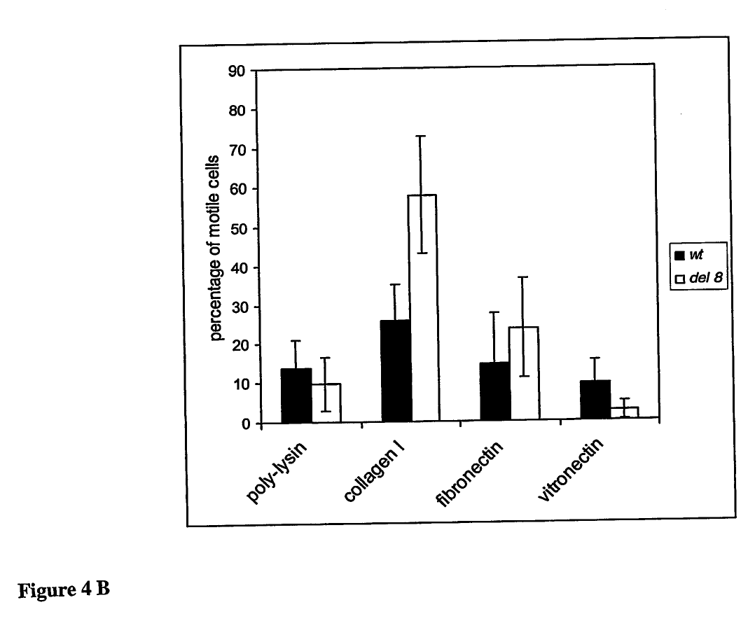

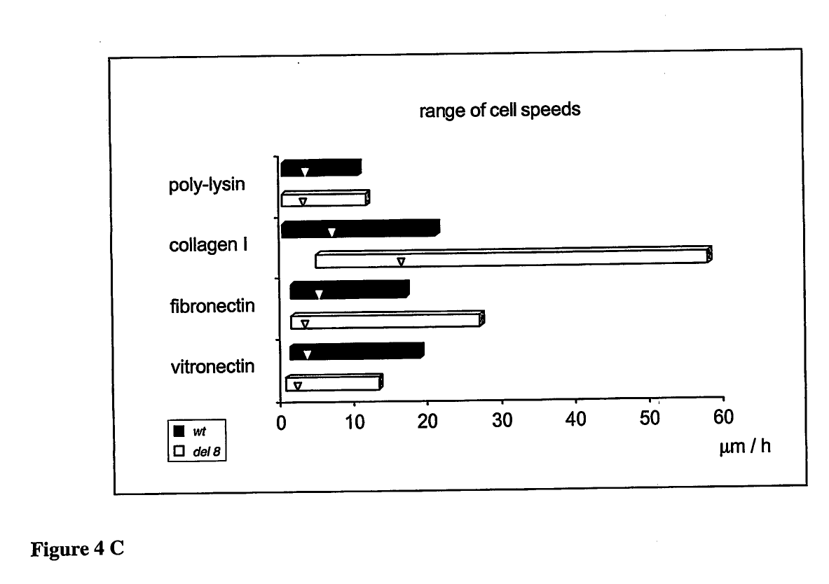

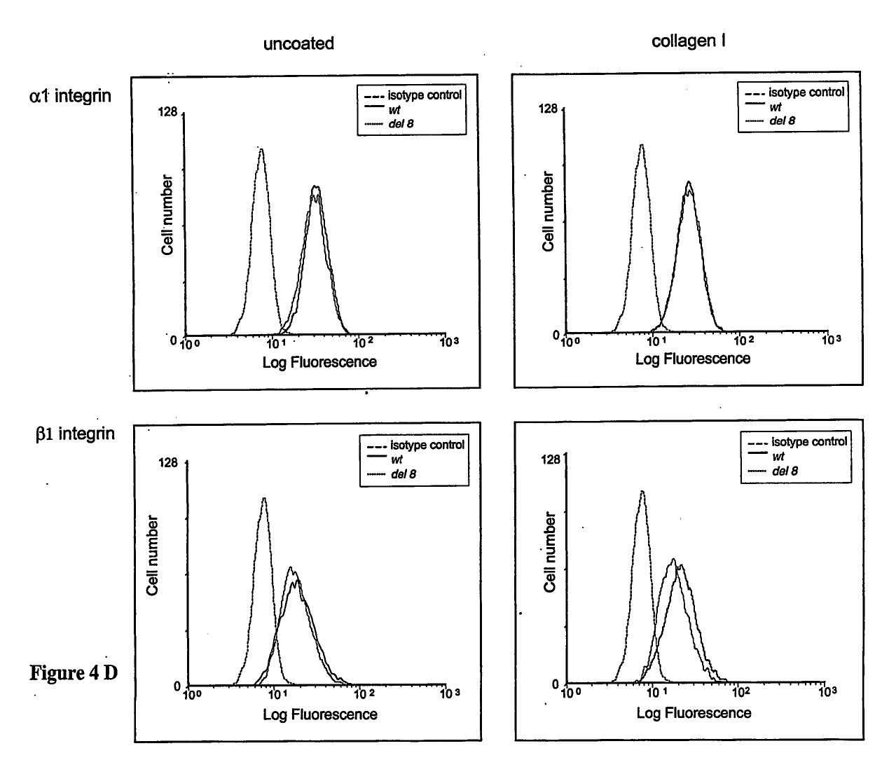

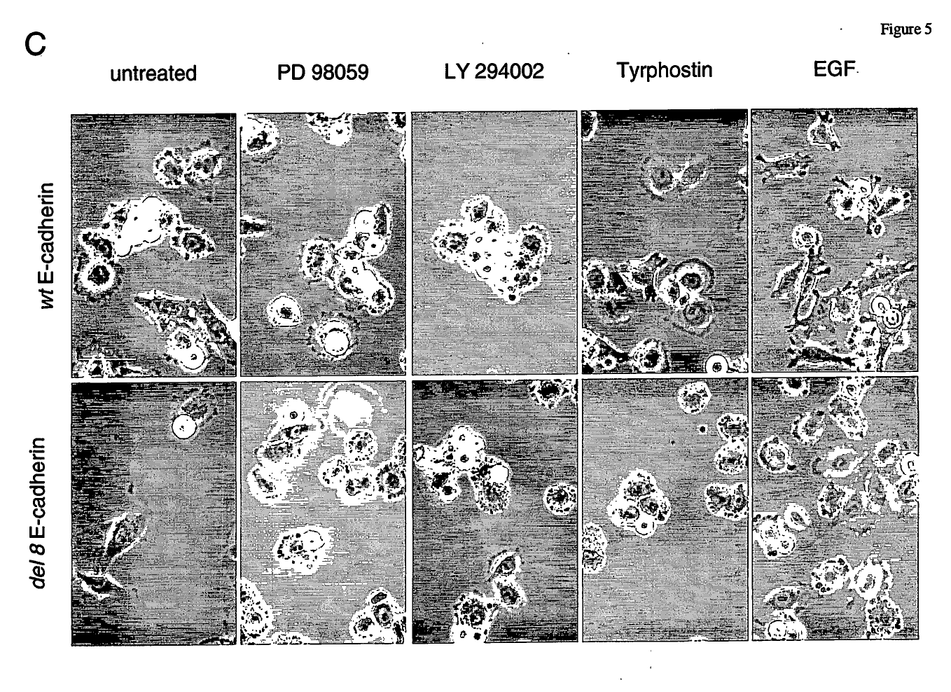

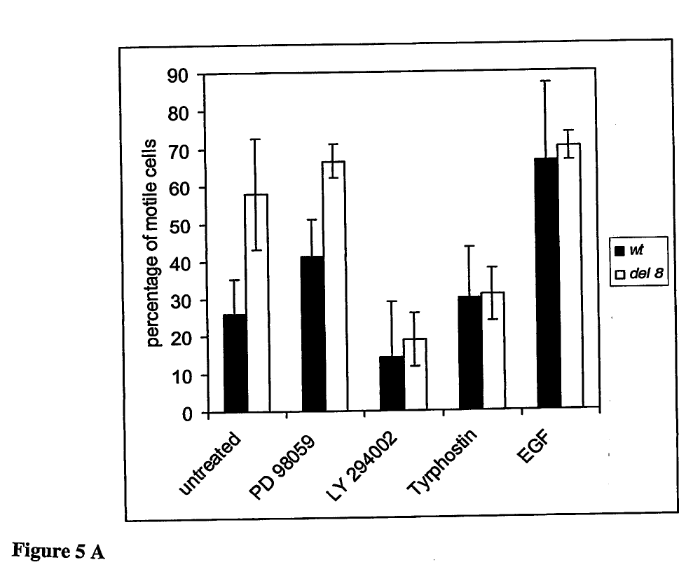

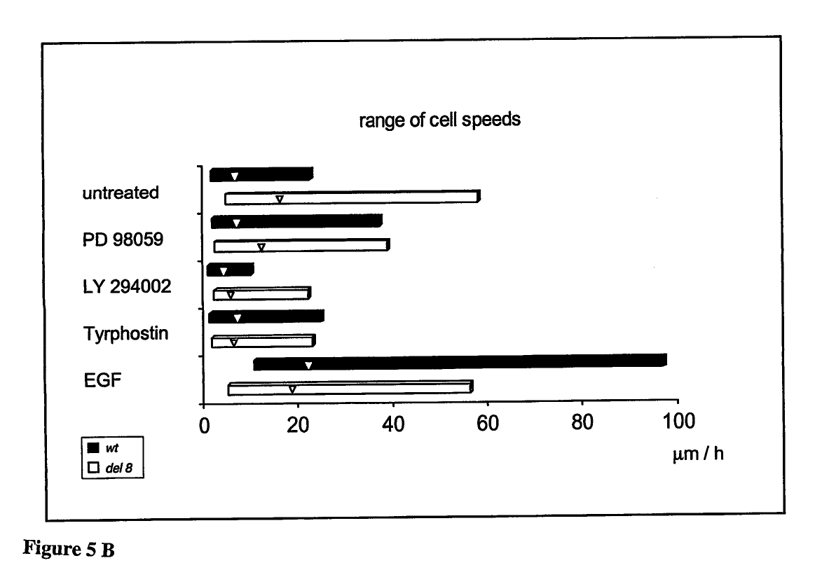

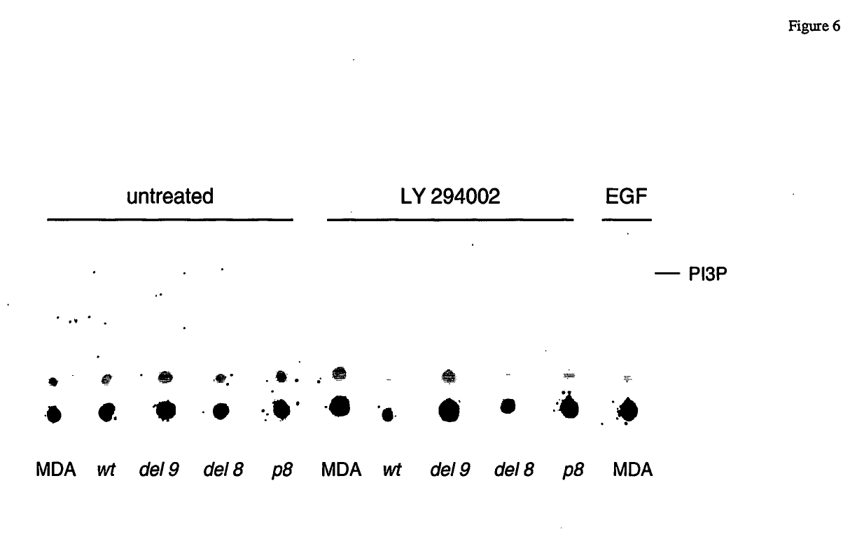

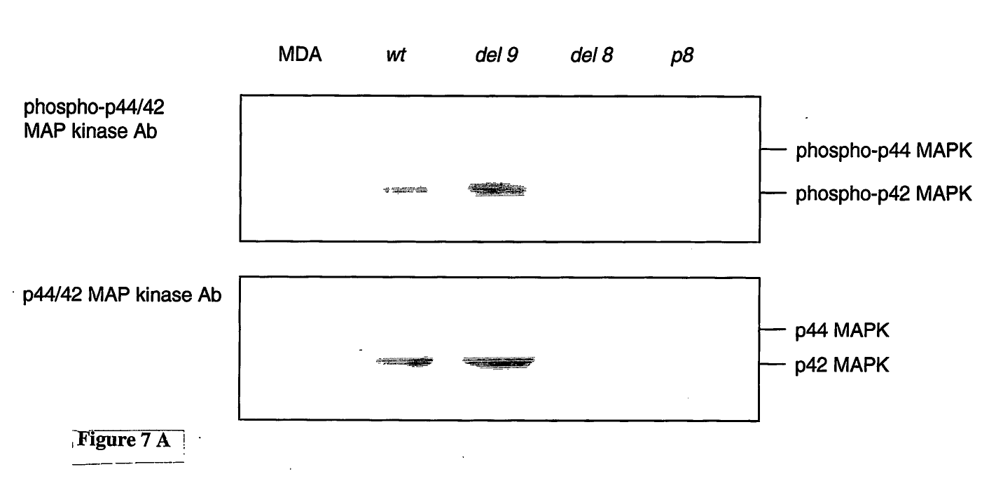

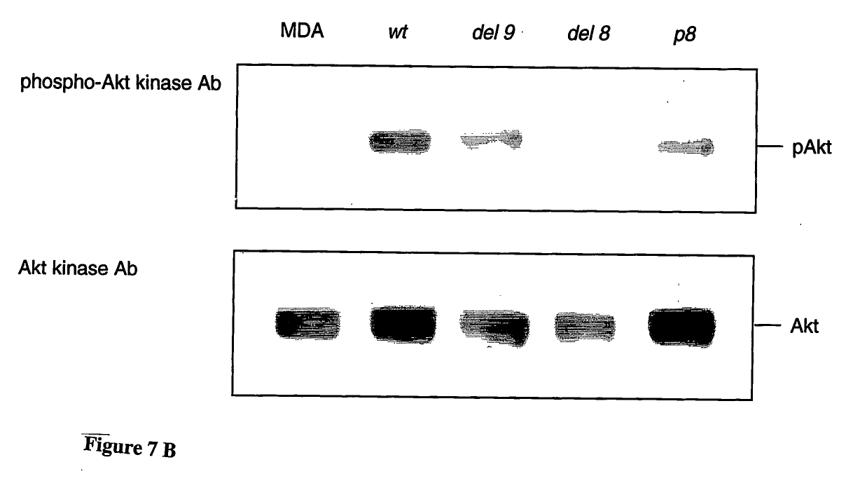

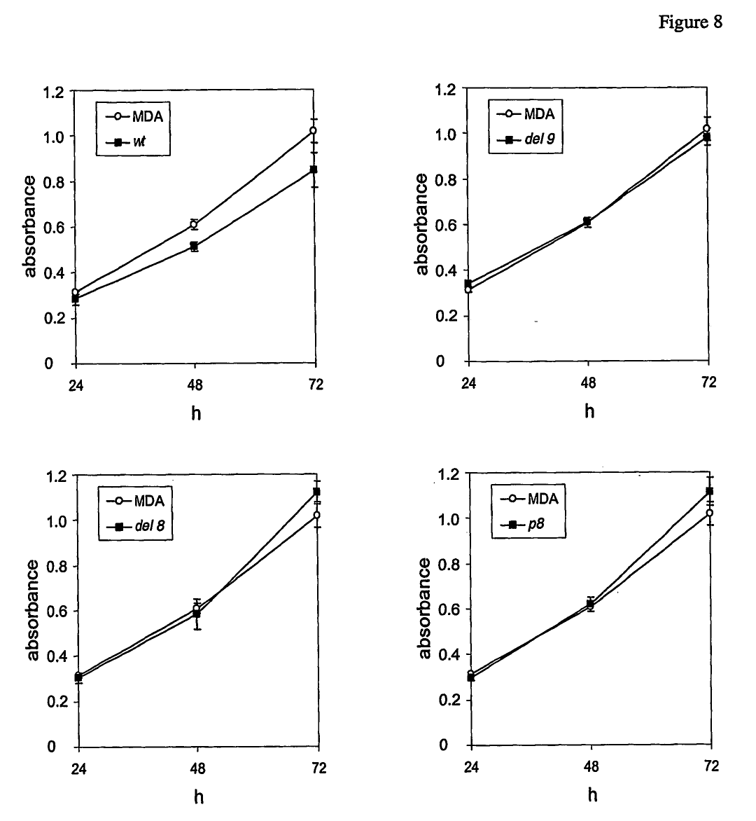

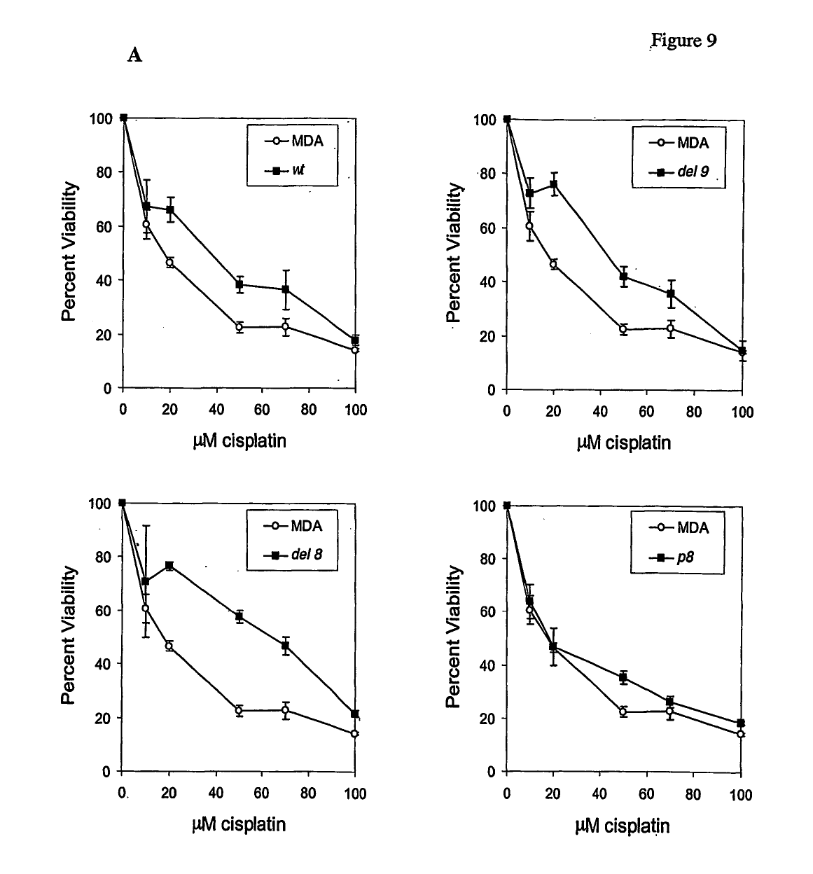

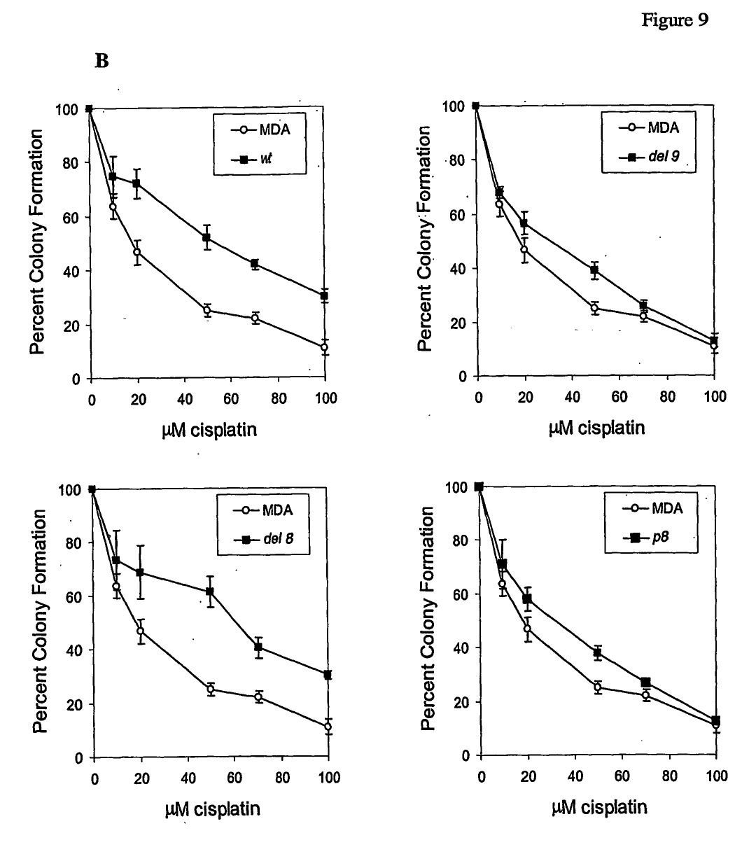

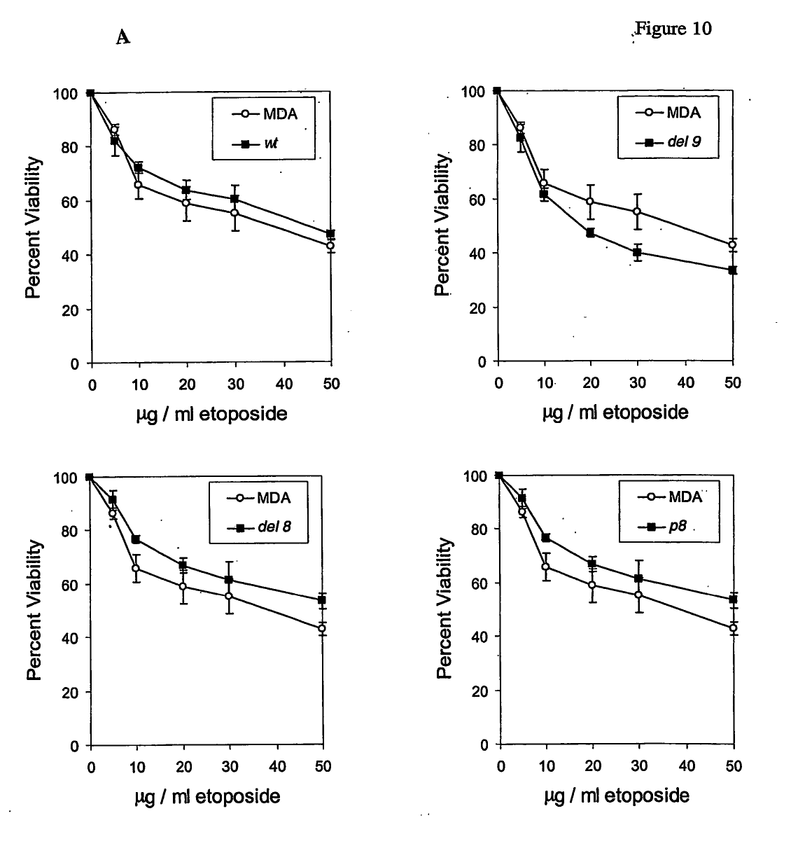

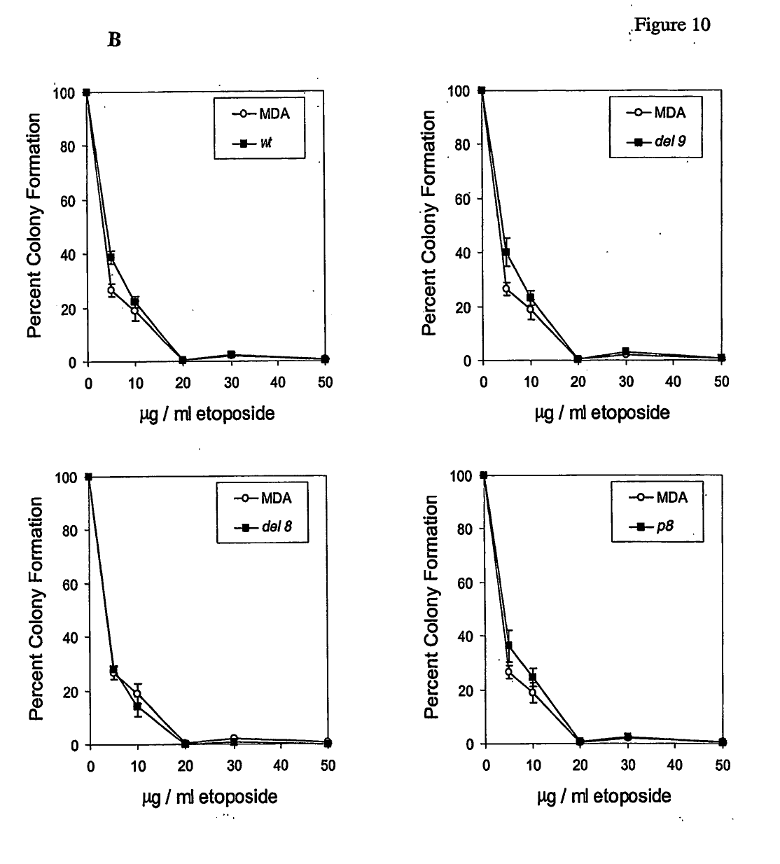

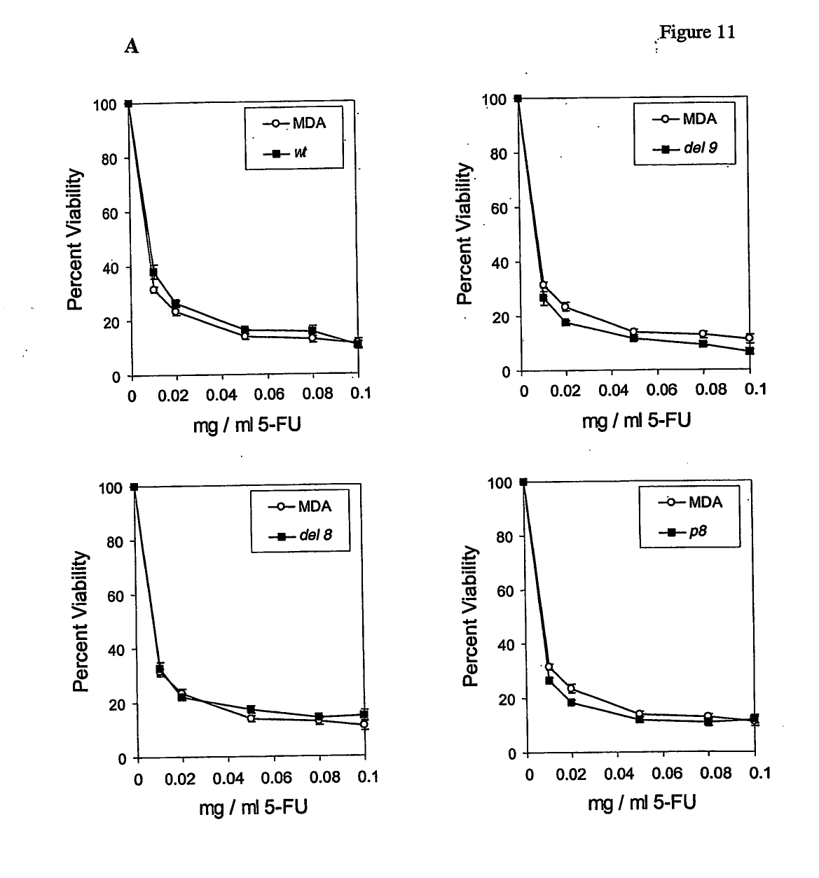

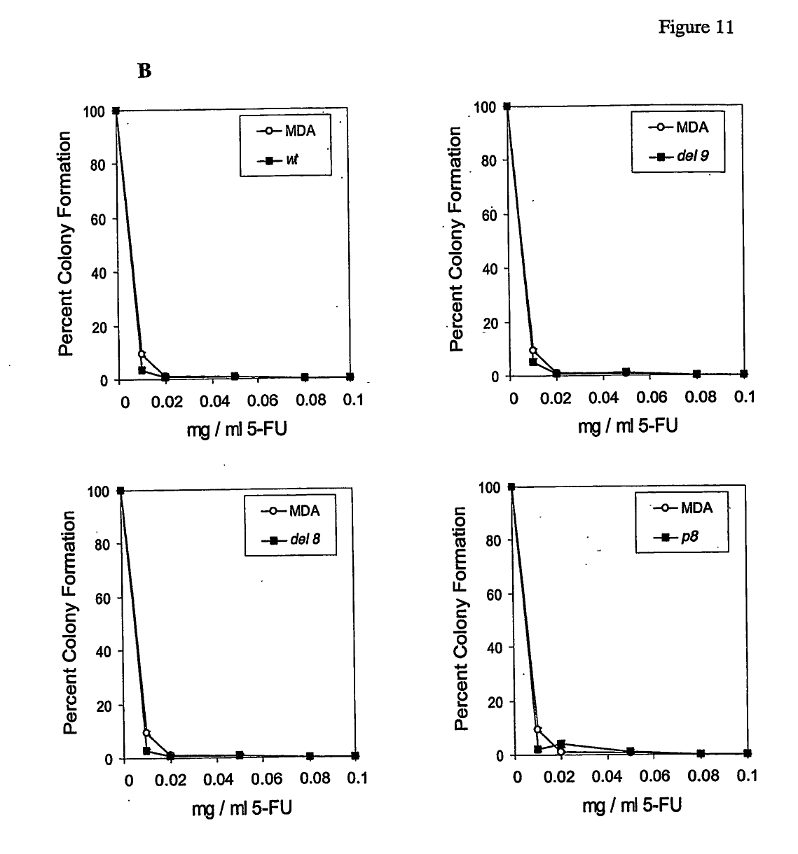

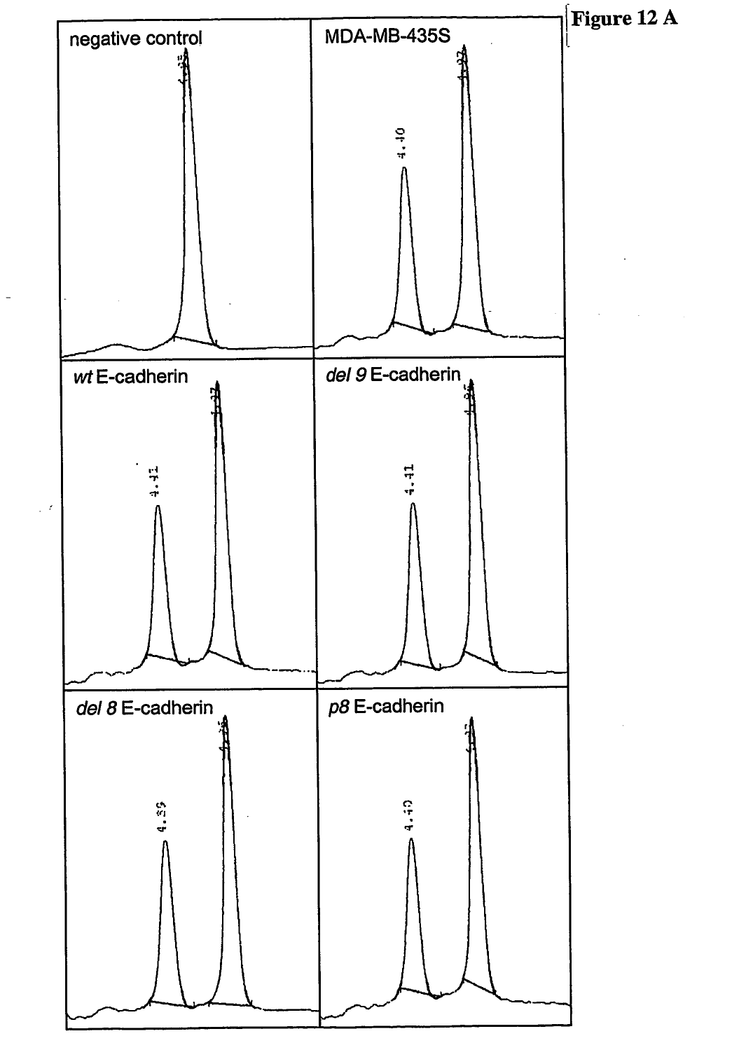

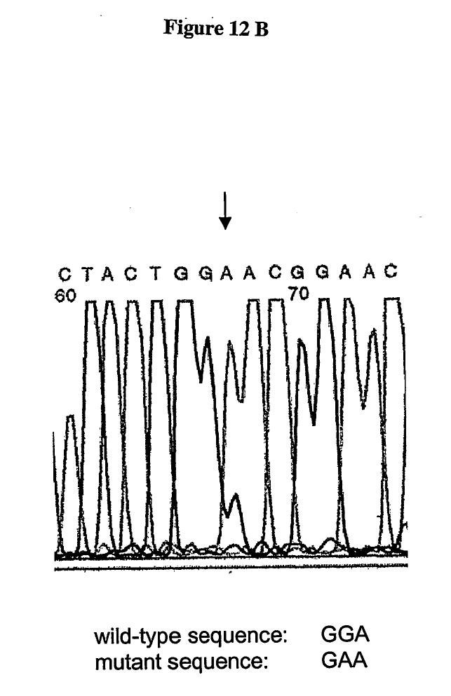

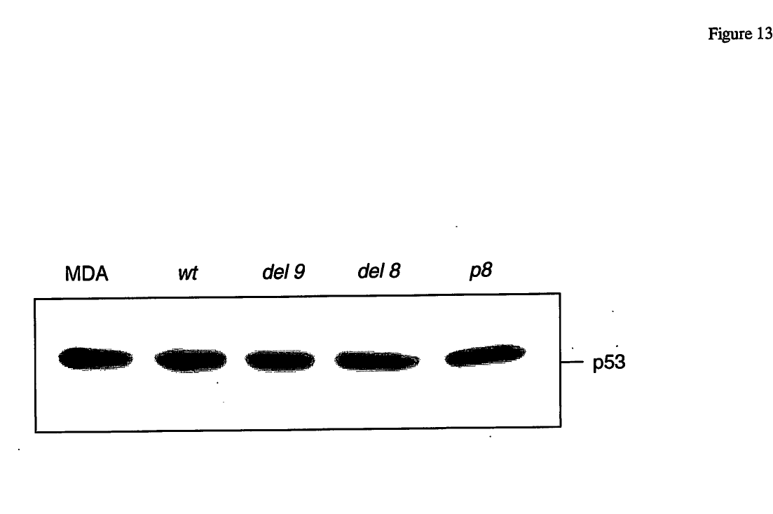

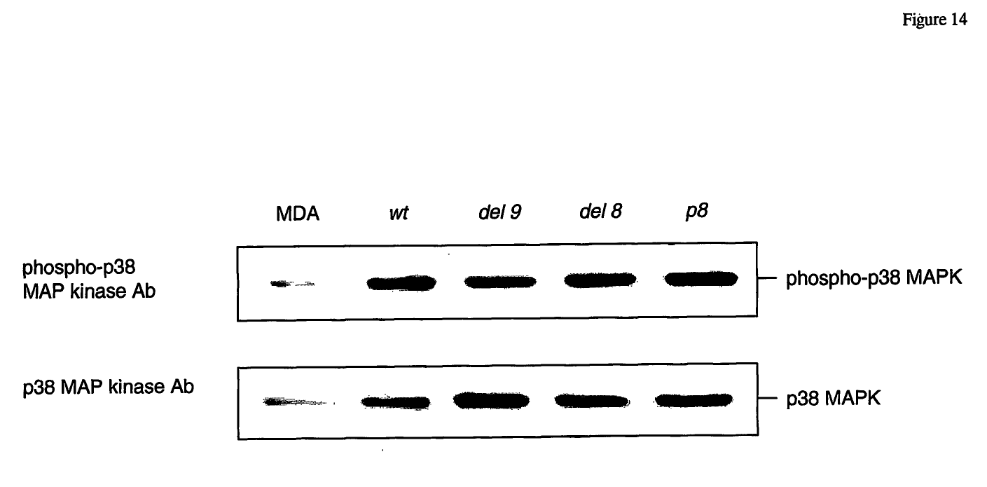

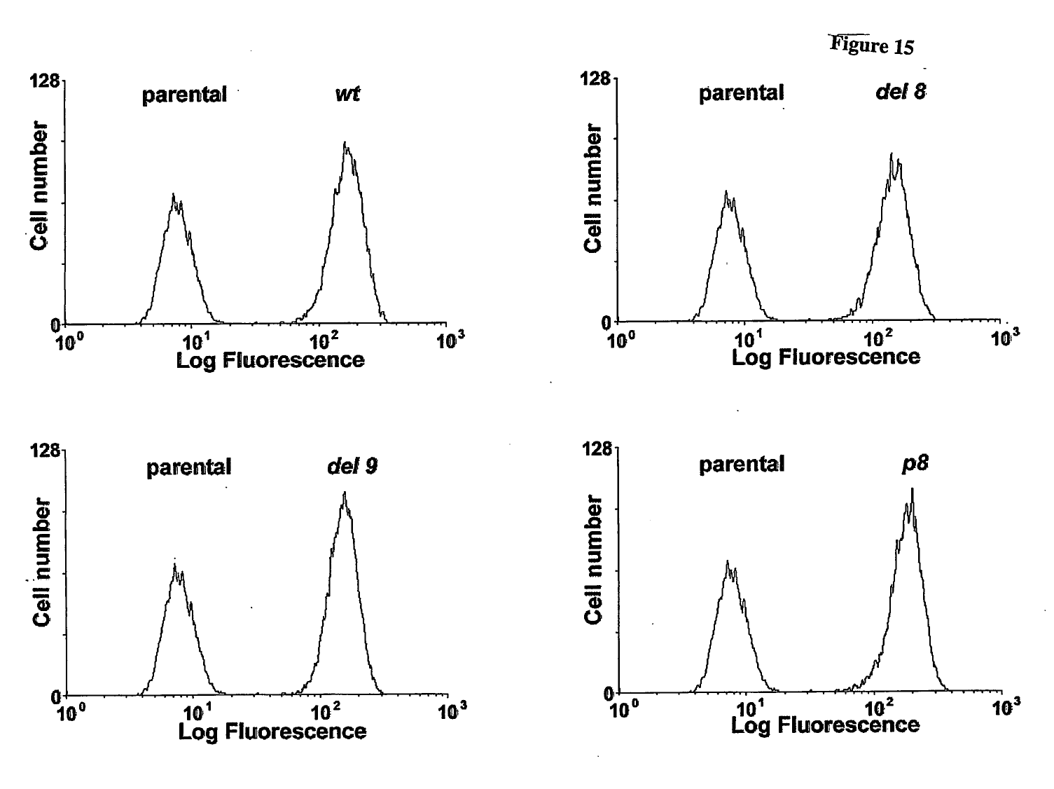

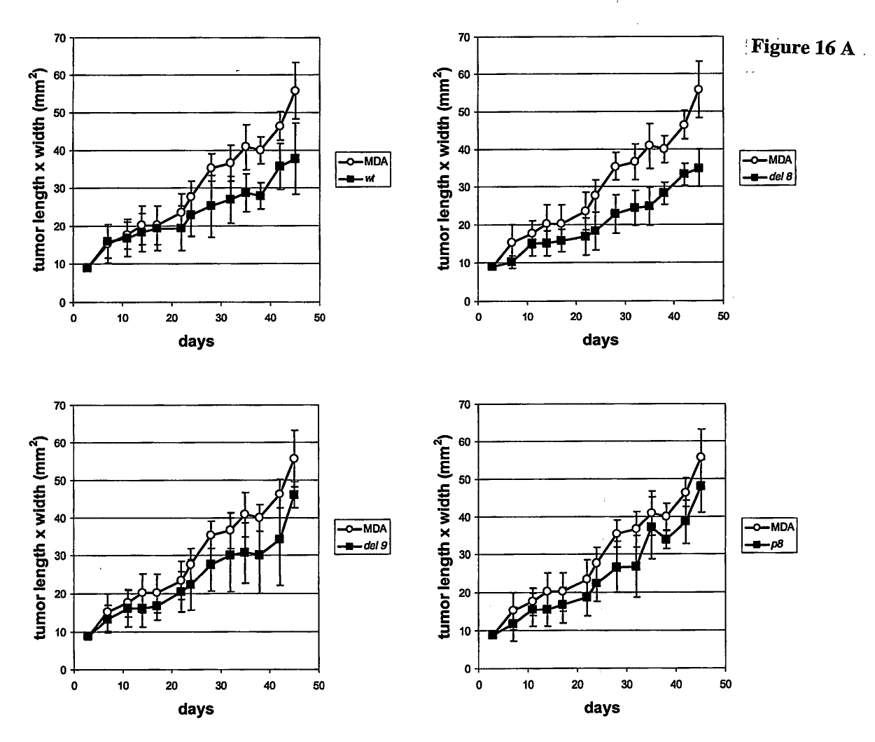

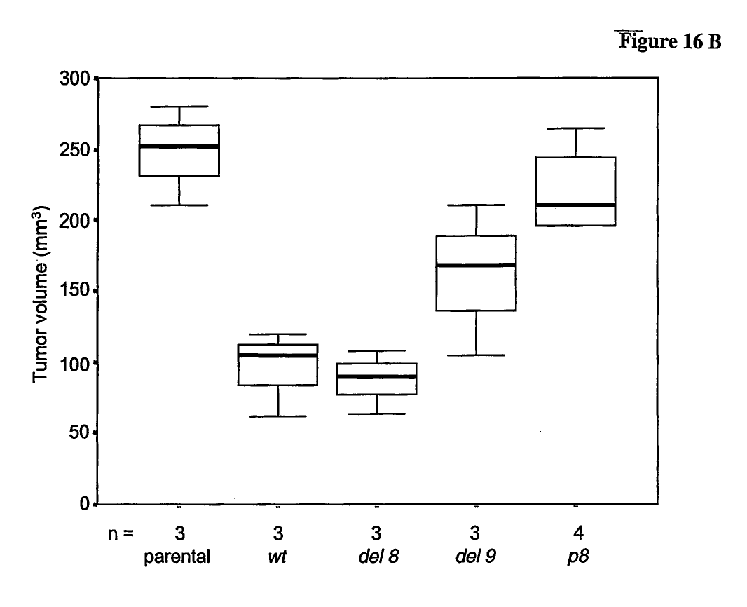

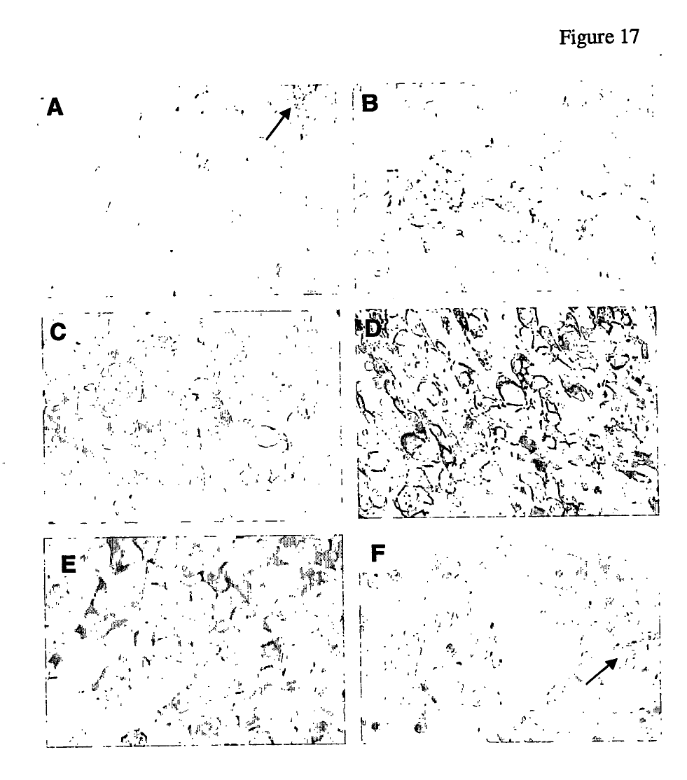

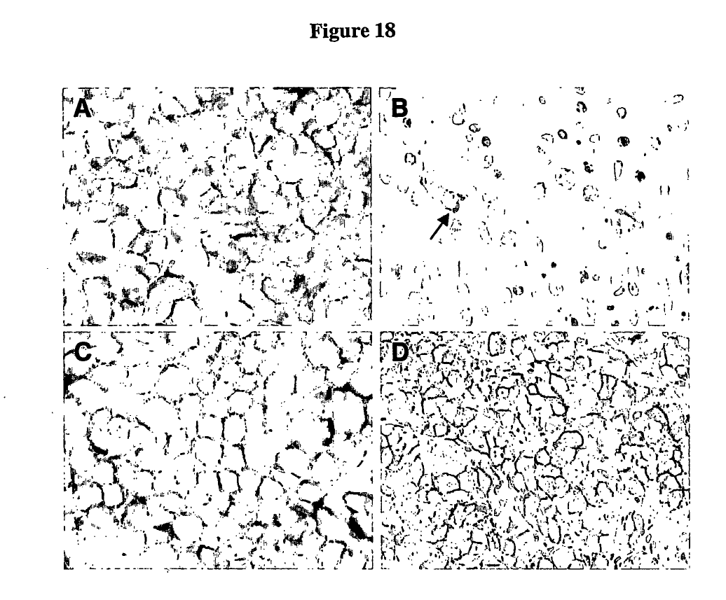

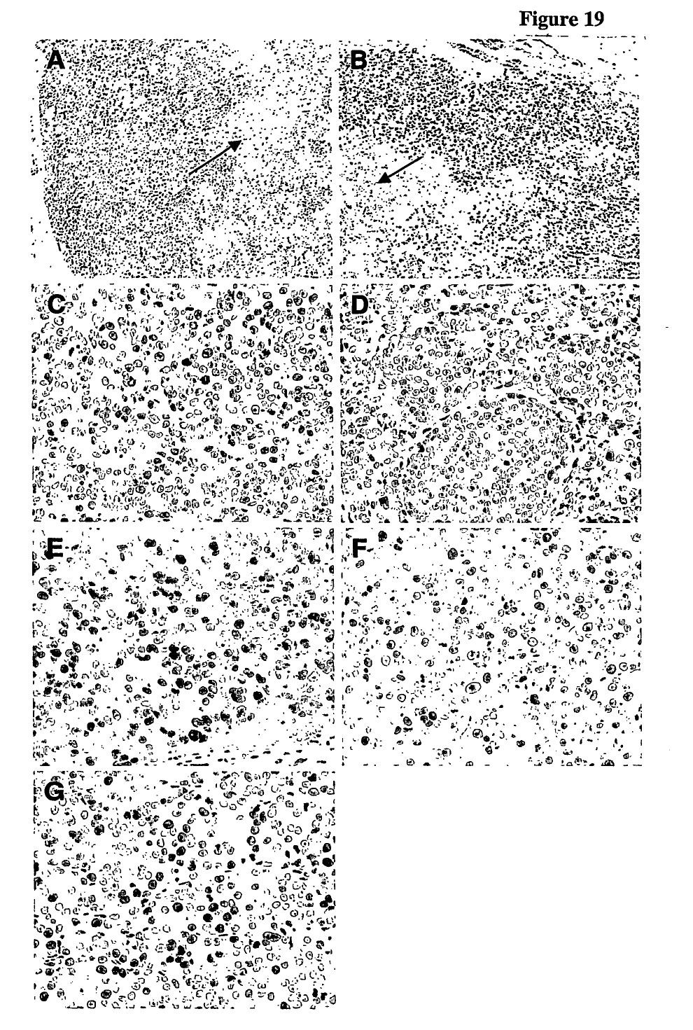

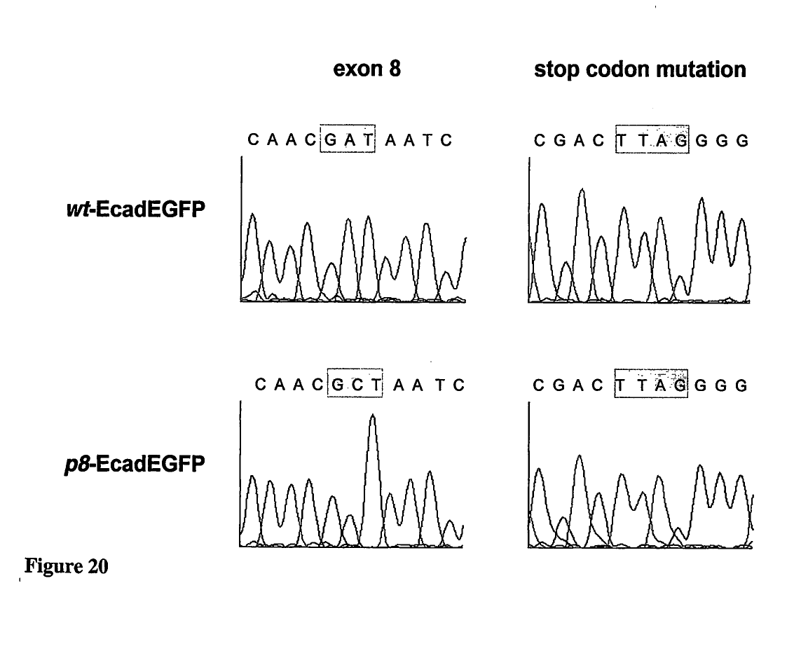

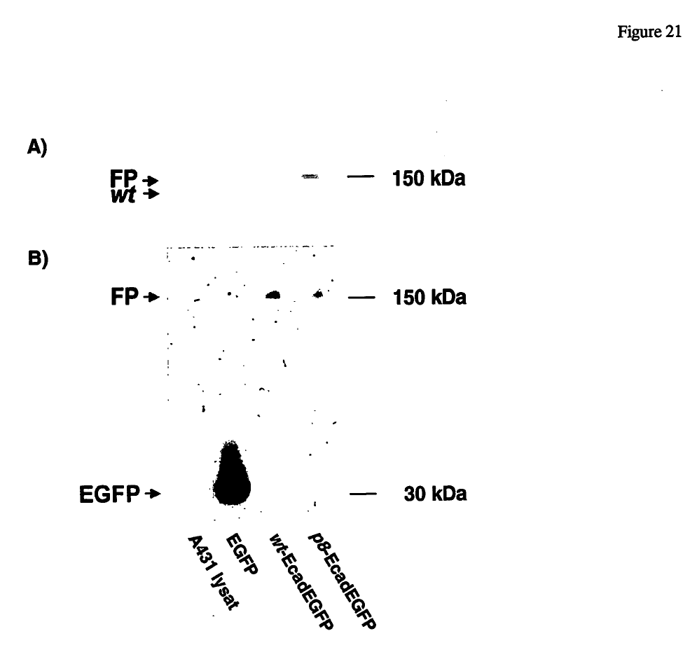

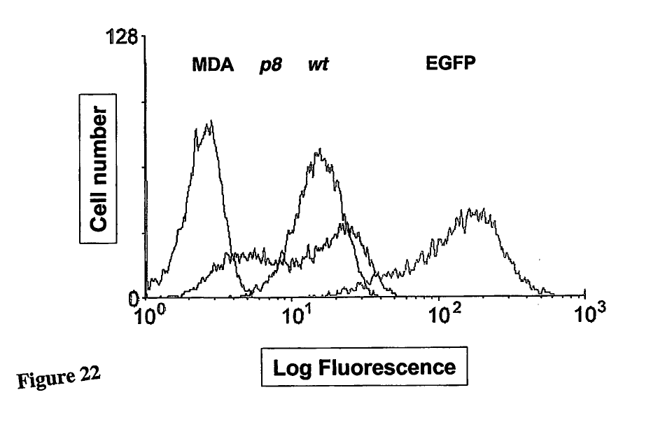

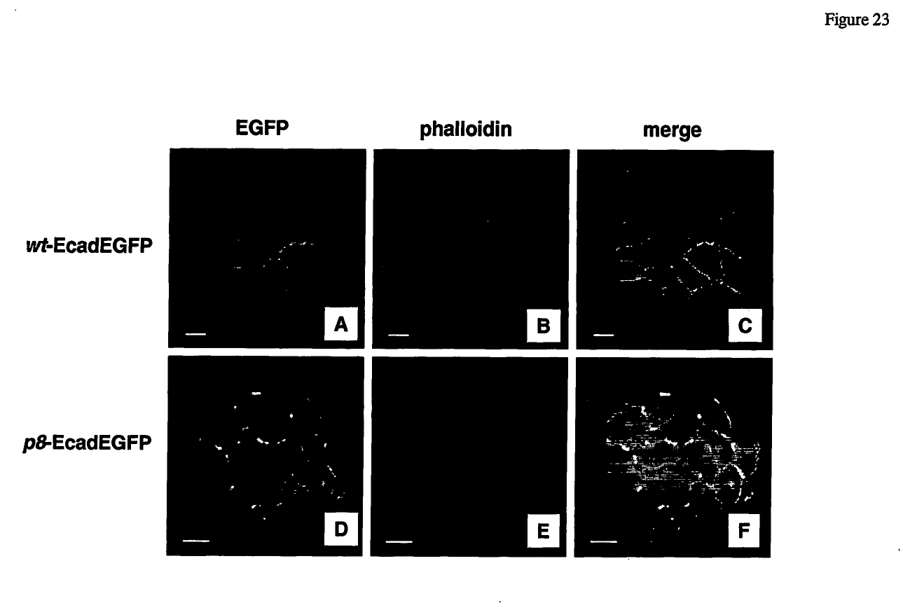

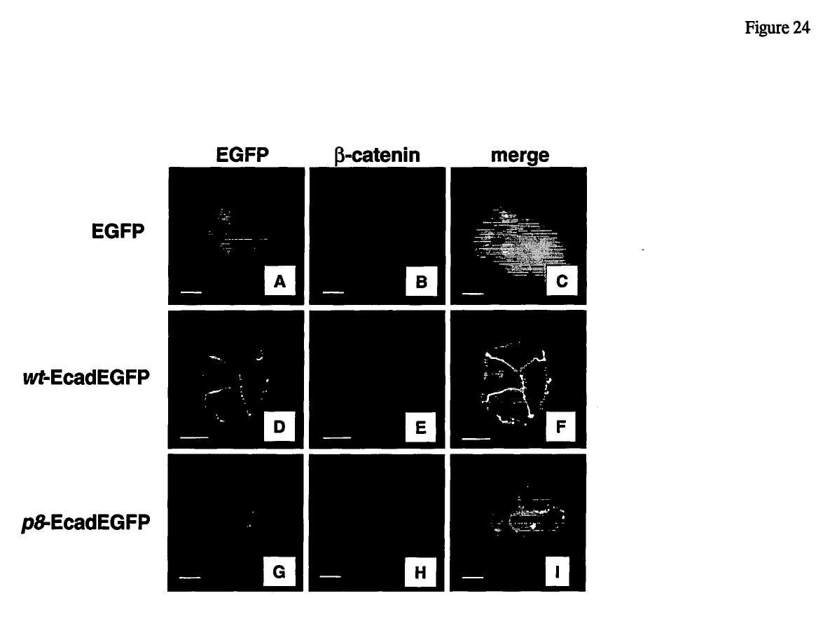

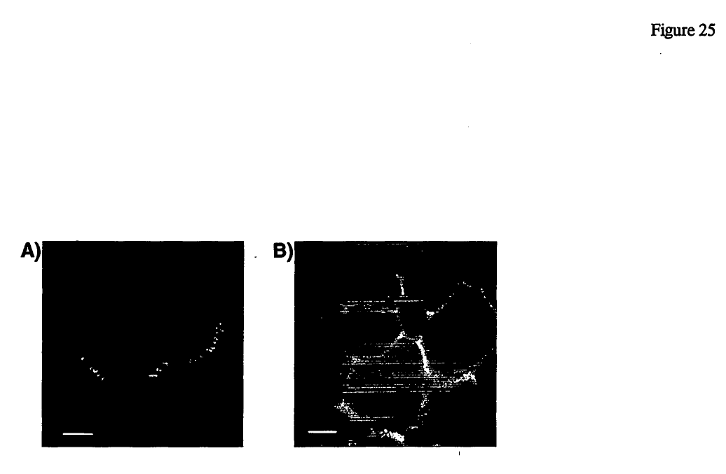

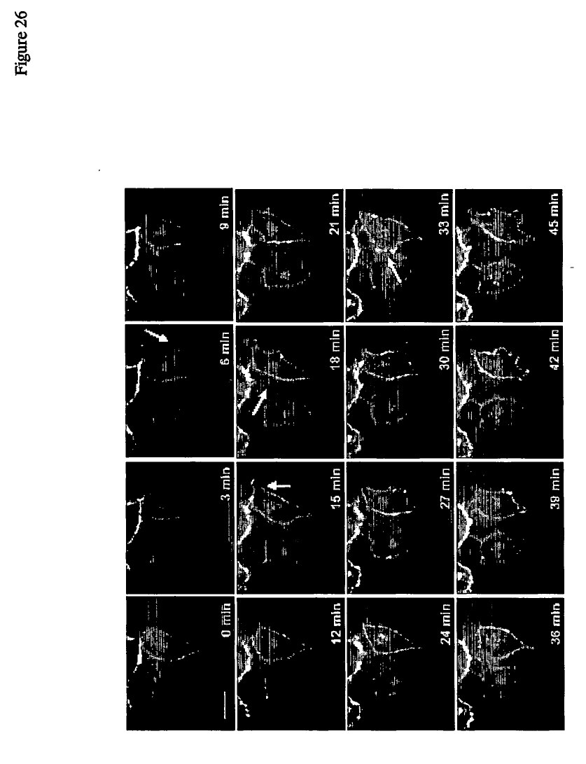

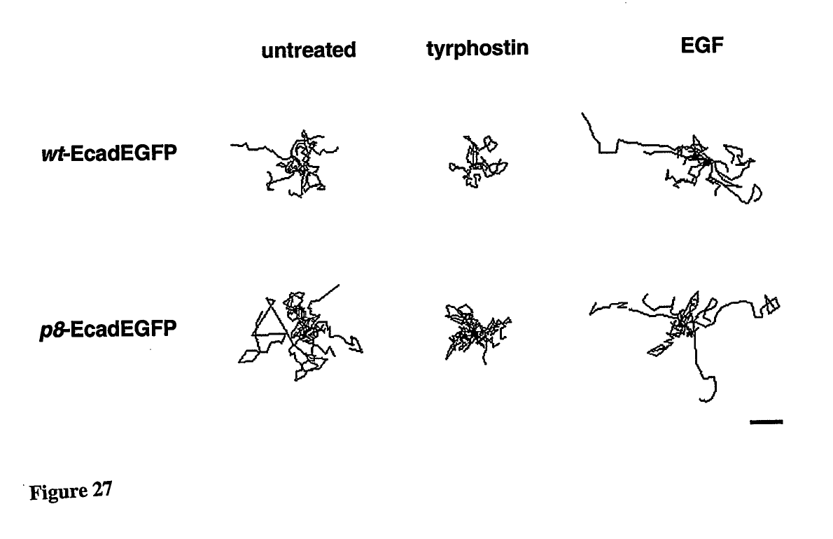

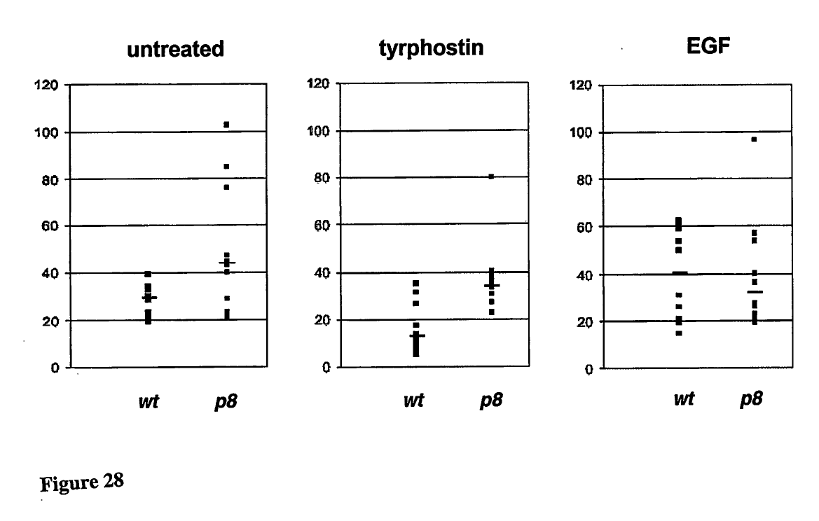

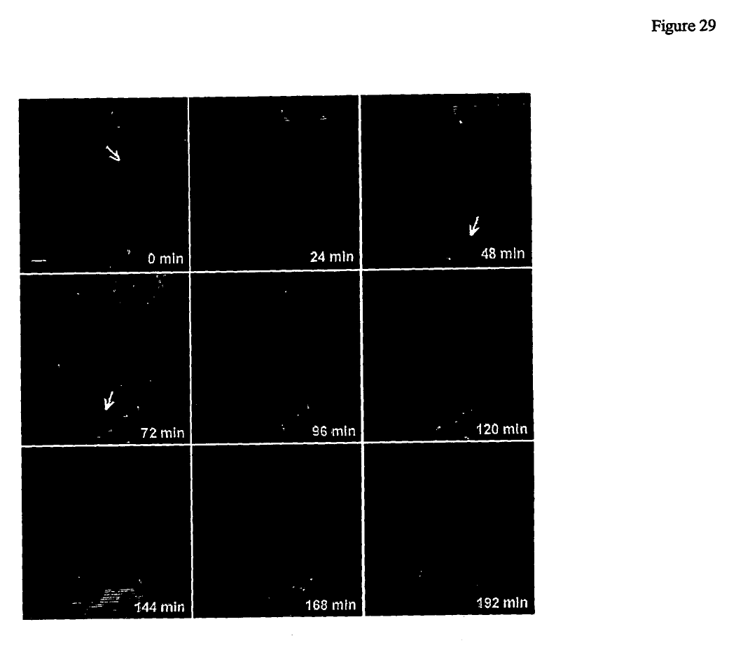

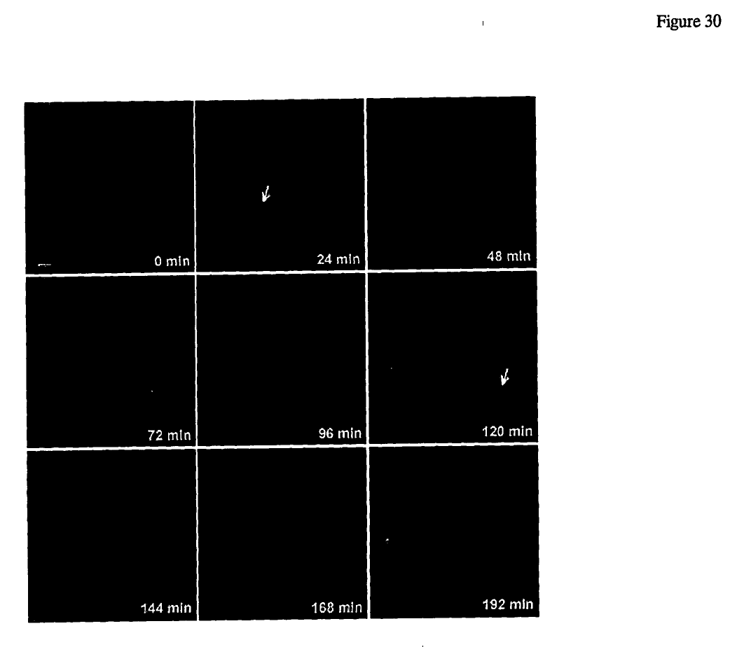

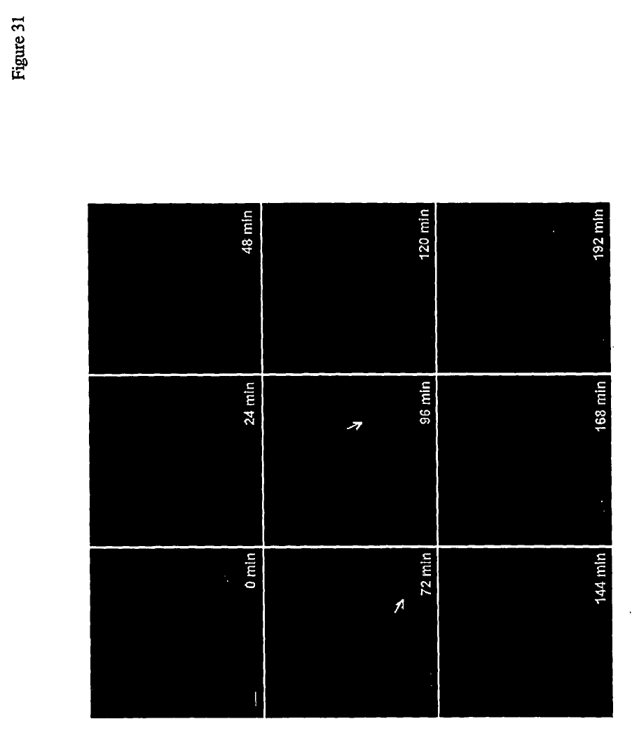

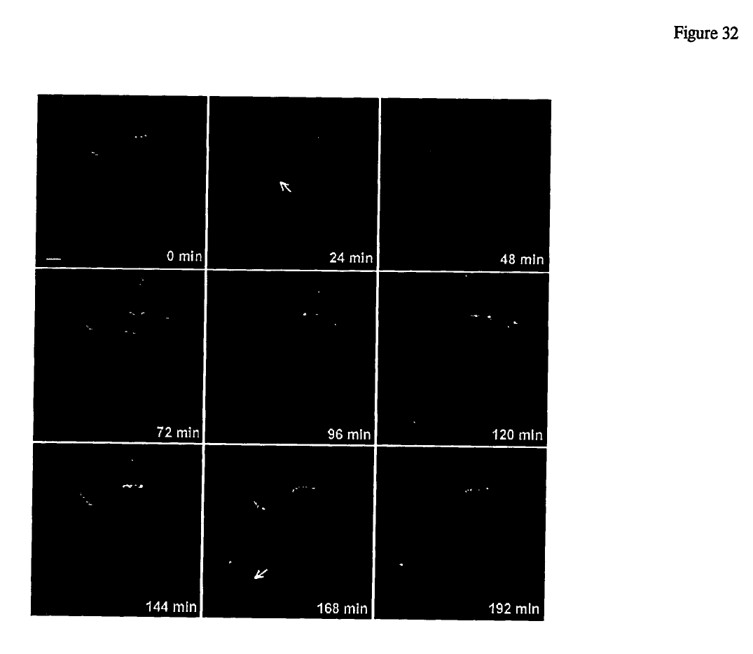

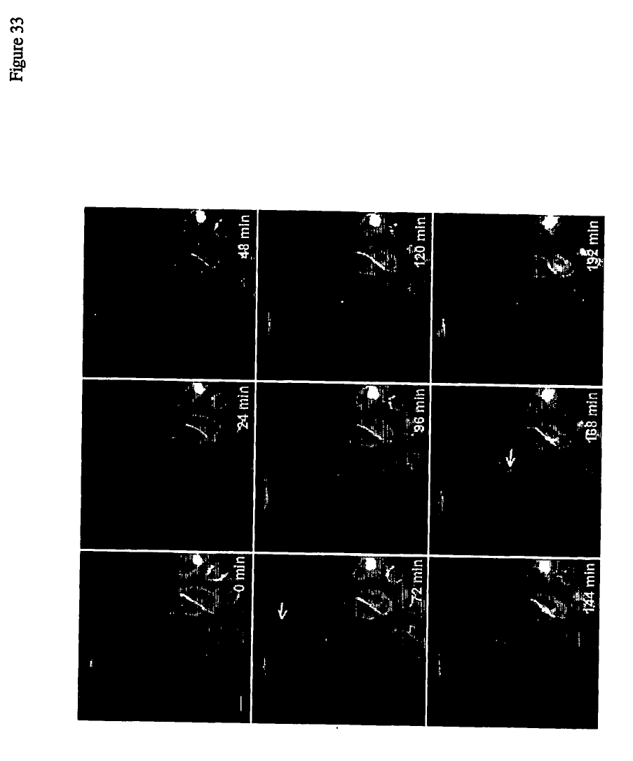

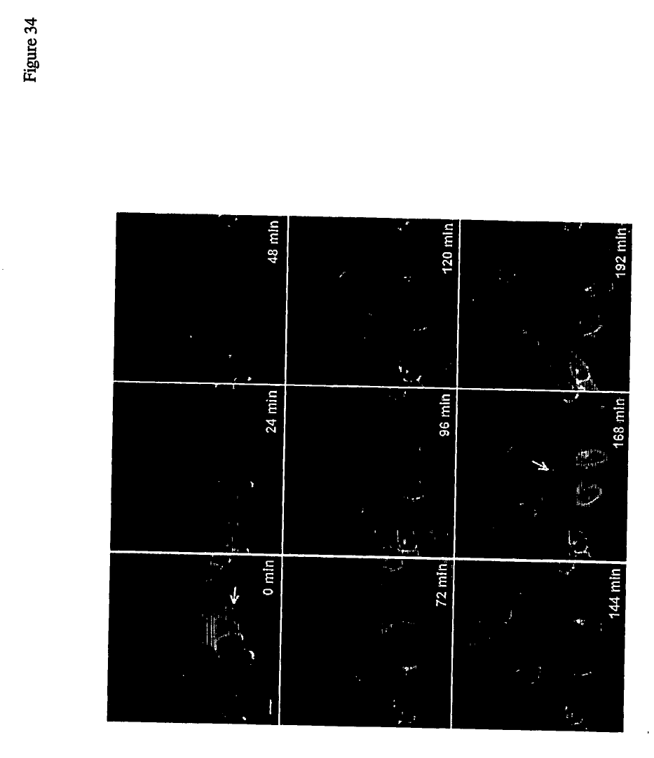

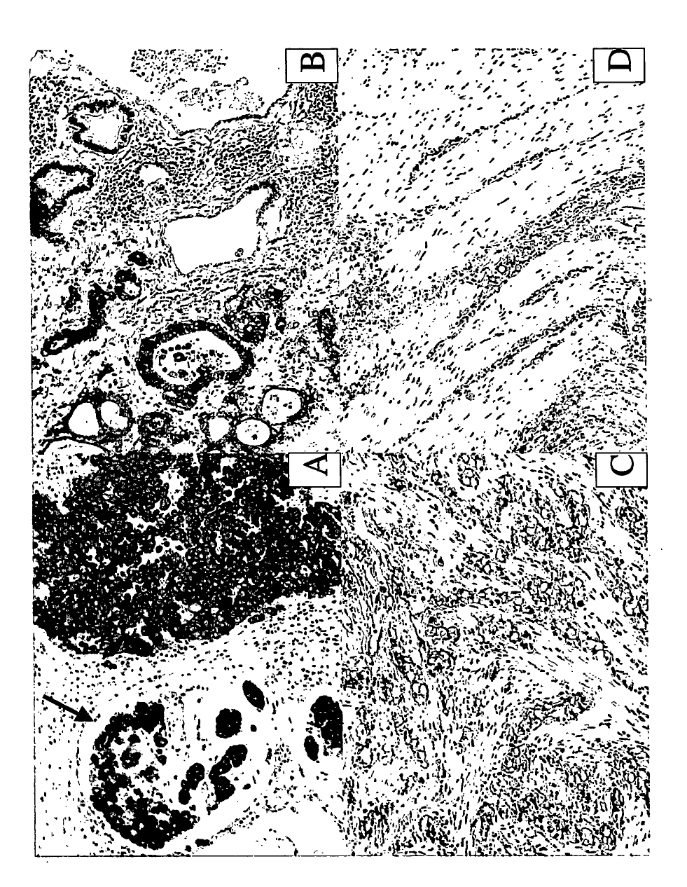

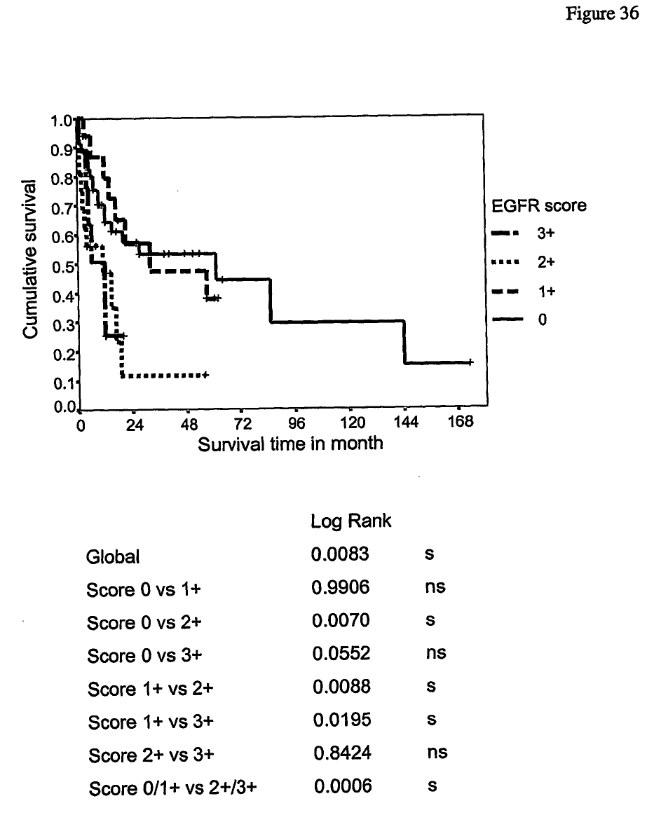

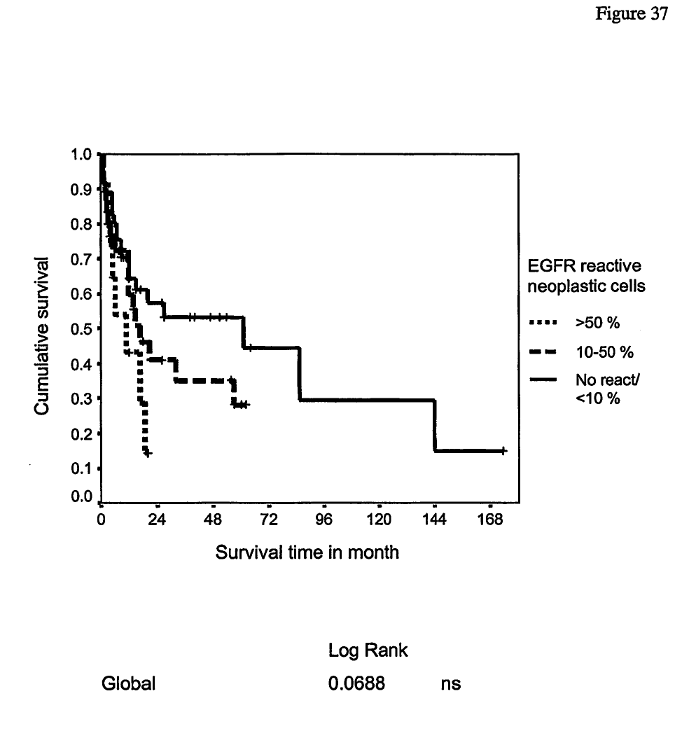

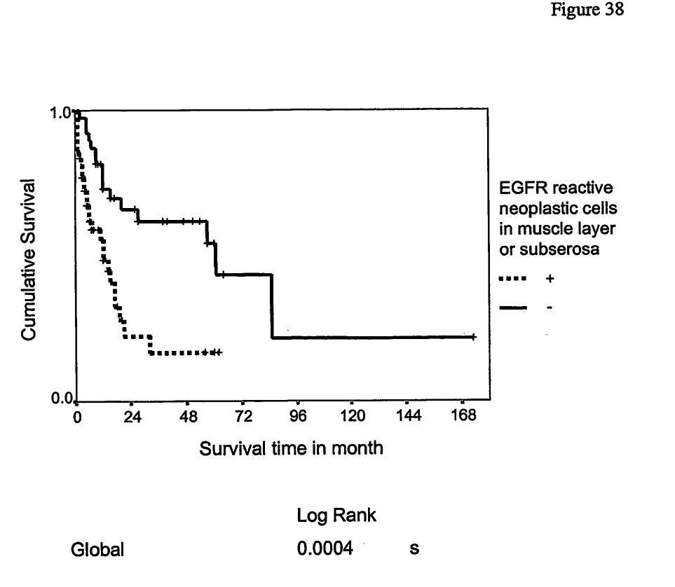

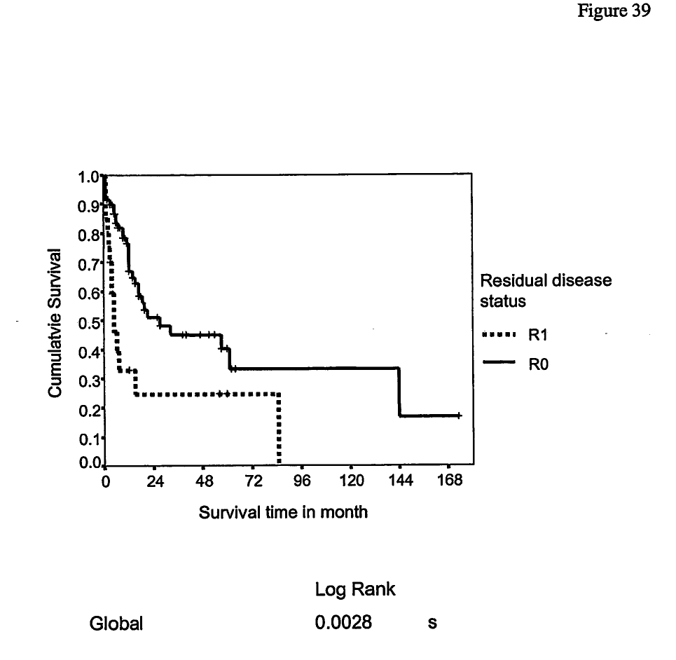

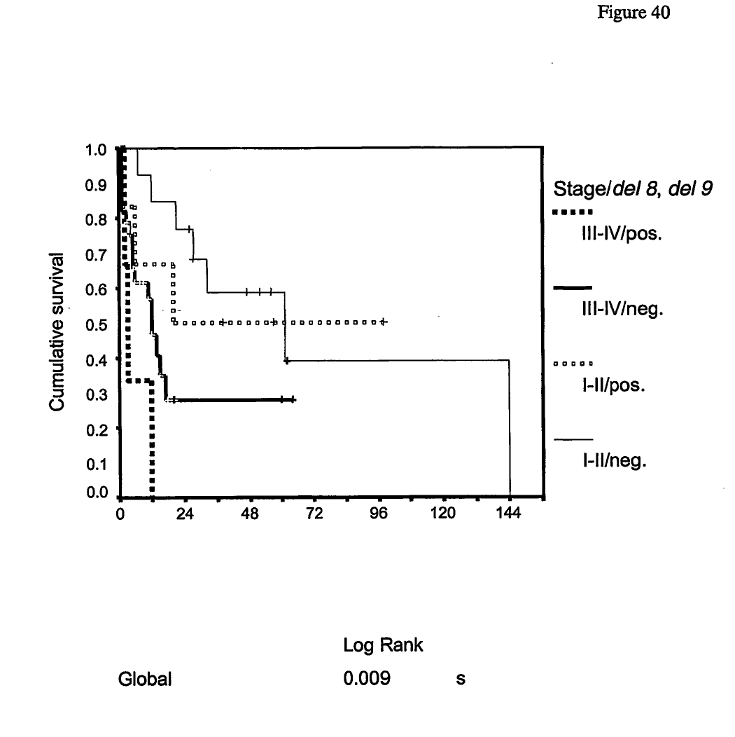

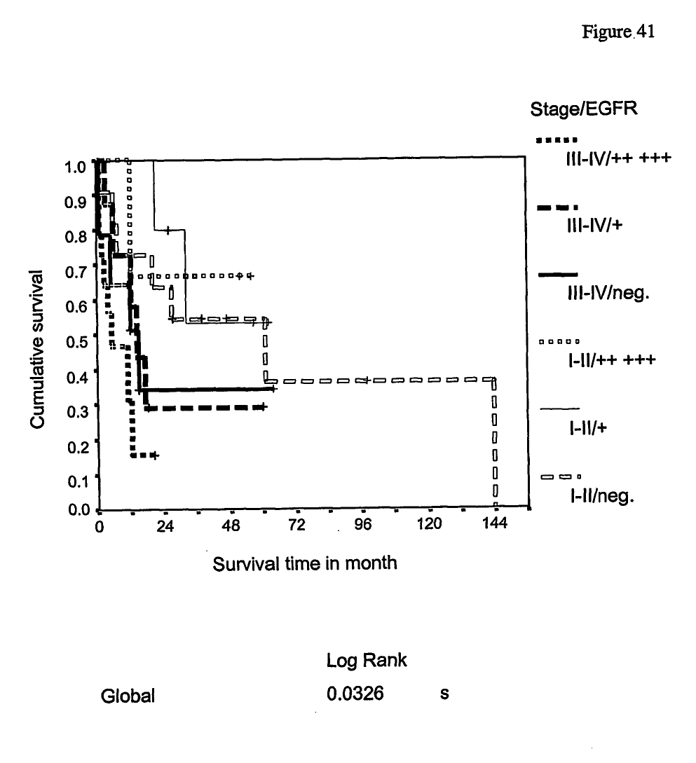

Immunoblot analysis performed with extracts from parental, wt or mutant E-cadherin expressing MDA-MB-435S cells using an antibody specific for p53 revealed that the p53 expression level was similar in all tested cell lines (FIG. 13). E-cadherin expression does not alter the p53 expression level.

Expression Level and Activity of the p38 Stress Activated Protein Kinase

Stress-activated kinase pathways transduce signals from a variety of stimuli including chemotherapeutics, irradiation, environmental changes, cytokines and growth factors. The p38 family of stress-activated kinases have been found to be involved in cell growth and differentiation, cell cycle and cell death (Ono, Cell Signal. 12 (2000), 1-13). To determine whether the expression level or activity of p38 were different in parental, wt or mutant E-cadherin expressing MDA-MB-435S cells, immunoblot analyses of cellular extracts were performed using an antibody specific for total p38 kinase or the phosphorylated, active form of p38. The expression level as well as the amount of phosphorylated p38 were induced by expression of wt and mutant E-cadherin to a similar extent (FIG. 14).

The above described experiments were undertaken to determine the effect of wt and mutant E-cadherin on cell proliferation and drug responsiveness to cisplatin, etoposide and 5-FU. The results suggest that the growth-supressive function of E-cadherin was impaired by tumor-associated E-cadherin mutations. Moreover, it was found that wt and mutant E-cadherin expressing MDA-MB-435S cells were less sensitive to cisplatin treatment than E-cadherin-negative, parental MDA-MB-435S cells. In contrast, treatment of parental, wt and mutant E-cadherin expressing MDA-MB-435S cells with 5-FU resulted in similar sensitivity profiles for all cell lines, as demonstrated by XTT and colony formation assay.

Although the mutant E-cadherin variants investigated in this study were cloned from diffuse-type gastric carcinomas, MDA-MB-435S mammary carcinoma cells were used instead of gastric carcinoma cells as recipient cells, because MDA-MB-435S cells have been widely used for studying E-cadherin function after transfection with E-cadherin expression constructs and are an established model system (Handschuh (1999), loc. cit.; Luber (2000), loc. cit.; Frixen, (1991), loc. cit.; Meiners (1998), loc. cit.). The parental MDA-MB-435S cell line lacks endogeneous E-cadherin due to methylation-associated silencing of E-cadherin gene expression (Graff (1995), loc, cit.) or downregulation of E-cadherin gene expression by snail (Cano (2000), loc. cit.), both mechanisms have been suggested. A deletion of exon 9 of E-cadherin, which is frequently detected in diffuse-type gastric carcinoma (Becker (1994), loc. cit.), was also found in the human breast cancer cell line MPE600 (Hiraguri, Cancer Res. 58 (1998), 1972-1977; Van de Wetering (2001), loc. cit.), indicating that investigation of E-cadherin mutations in breast cancer cells is of physiological relevance.

The data demonstrate that mutations in the extracellular domain of E-cadherin, which affect the adhesive function of the molecule, also interfere with its growth-suppressive function. It has been shown recently that E-cadherin regulates cell growth by modulating the transcriptional activity of β-catenin (Stockinger, J. Cell Biol. 154 (2001), 1185-1196). Besides its role in E-cadherin-mediated cell adhesion, β-catenin forms nuclear complexes with high mobility group transcription factors (Behrens, Nature 382 (1996), 638-642; Huber, Mech Dev 59 (1996), 3-10; Molenaar, Cell 86 (1996), 391-399). The growth-suppressive effect of E-cadherin required the presence of its cytoplasmic β-catenin interaction domain and/or correlated strictly with the ability to negatively interfere with β-catenin transcriptional activity (Stockinger (2001), loc. cit.).

The mutant E-cadherin variants investigated in the present study have intact α-catenin binding sites and form complexes with β-catenin (Luber (2000), loc. cit.). However, the E-cadherin mutations induce partially abnormal cytoplasmic and perinuclear β-catenin staining, possibly because the mutant E-cadherin variants show the same abnormal staining pattern (Luber (2000), loc. cit.). Whether the mislocalization of β-catenin correlates with the transcriptional activity in our cell lines needs to be investigated. In any case, since β-catenin signaling regulates expression of c-myc (He, Science 281 (1998), 1509-1512) and cyclin Dl (Shtutman, Proc. Natl. Acad. Sci. USA 96 (1999), 5522-5527; Tetsu, Nature 398 (1999), 422-426), an increase in β-catenin signaling in the mutant E-cadherin expressing MDA-MB-435S cell lines as compared to the wt E-cadherin expressing cells could explain why the former cells have a higher proliferate rate. Disregulation of cell growth in tumors with E-cadherin mutations may be advantageous to tumor cells.

In the present study, a difference in chemosensitivity to the anticancer drug cisplatin was detected between parental MDA-MB-435S cells and transfected cells expressing wt or mutant E-cadherin. Cisplatin is a platinum-containing, DNA damaging agent which is effective against solid tumors (Pinto, Biochim. Biophys. Acta. 780 (1985), 167-180). Cisplatin exposure leads to the formation of intrastrand cross-links. Several genes have been identified that mediate sensitivity to cisplatin (Niedner, Mol. Pharmacol. 60 (2001), 1153-1160), for instance DNA mismatch repair genes and hMSH2 and its heterodimer partners bind to cisplatin-DNA adducts. Defects in DNA mismatch repair genes produce resistance to cisplatin.

In contrast to the results obtained with cisplatin, the chemosensitivity of parental, wt and mutant E-cadherin expressing cells to etoposide and 5-FU was similar in all cell lines, independent of the expression or mutation status of E-cadherin. A possible explanation is that these drugs act by different mechanisms: Etoposide is an inhibitor of the enzyme DNA topoisomerase II which is essential for DNA replication, transcription, chromosomal segregation and DNA recombination (Hande, Eur. J. Cancer 34 (1998), 1514-1521); 5-FU acts as a competitive inhibitor of thymidylate synthase and blocks both RNA and DNA synthesis (Parker, Pharmacol. Ther. 48 (1990), 381-395).

Genetic abnormalities of the p53 tumor suppressor gene are among the most frequent mutations in tumorigenesis (Prives, J. Pathol. 187 (1999), 112-126). p53 protects cells from DNA damage by inducing either growth arrest or apoptosis in response to stress signals (Levine, Cell 88 (1999), 323-331). In response to cellular stress or DNA damage, p53 becomes activated and functional. A previous study has suggested a correlation between the p53 mutation status and growth inhibition of anticancer drugs in 60 cell lines of the National Cancer Institute (O'Connor (1997), loc. cit.). In light of these findings, we investigated the genetic p53 background and p53 expression level of wt and mutant E-cadherin MDA-MB-435S transfectants. Investigation of exon 5-8 by DHPLC and Western blot analysis revealed that the p53 mutation status and expression level were unaffected by wt or mutant E-cadherin. The presence of one wt p53 allele certainly influences the chemosensitivity of MDA-MB-435S cells, since cells which are heterozygote for p53 have been shown to be more sensitive to treatment with chemotherapeutic drugs than p53-deficient cells (Bunz, J. Clin. Invest. 104 (1999), 263-269).

In the present study, expression and activity of p38 kinase were increased by wt and mutant E-cadherin. This result is of relevance for cell survival, since p38 kinase has been implicated the reaction of cells to genotoxic stress induced by chemotherapeutic agents. For instance, the p38 kinase has been shown to play a key role in the activation of p53 by cisplatin (Sanchez-Prieto, Cancer Res. 60 (2000), 2464-2472). p38 associates physically with p53, and phosphorylates the NH2-terminal transactivation domain of p53, thereby stimulating its functional activity. Inhibition of the p38 kinase also diminished the apoptotic fraction of cells exposed to chemotherapeutic agents and increased cell survival, thus suggesting a role for p38 activation in the apoptotic response to genotoxic stress. Involvement of p38 in apoptosis-associated membrane blebbing and nuclear condensation and a role of p38 kinase in sensitizing cells to killing by chemotherapeutic agents have also been demonstrated in other studies (Pillaire, Biochem. Biophys. Res. Commun. 278 (2000), 724-728; Deschesnes, Mol. Biol. Cell 12 (2001), 1569-1582).

Genetic alterations play a causative role in tumor formation and progression. Specific genetic alterations might also determine the patient's outcome after chemotherapeutic treatment. For example, it has been shown that p53 alters the chemosensitivity of cells. In the present study, it is documented that the presence of E-cadherin alters the sensitivity against cisplatin. Since alterations of E-cadherin found in tumors are mutational inactivation and transcriptional downregulation (Van Aken, Virchows Arch. 439 (2001), 725-751), these results are of interest with regard to chemotherapeutic treatment of patients with abnormalities in the E-cadherin status. Accordingly, the present invention provides for the use of (an) EGF receptor antagonist(s)/inhibitor(s) in the medical intervention and/or prevention of gastric cancers, preferably of diffuse gastric carcinomas. The results documented herein above demonstrate that said EGF receptor antagonist may be employed in co-therapy approaches with further anti-cancer drugs useful in chemotherapeutic intervention of cancers with modifications in the E-cadherin status. The results and experiments provided herein demonstrate means and methods how EGF-receptor antagonists/inhibitors or chemotherapeutics may be tested for efficacy in a cell culture system, namely in carcinoma cell transfectants expressing wildtype or mutant E. cadherin molecules. Preferably said wildtype E-cadherin expressing carcinoma cell line is compared in its physiological, biochemical and/or morphological behavior to the carcinoma cell line expressing at least one mutant E-cadherin. It is also envisaged that these tests (screening. and/or assay systems) may comprise the use of other cells which are transfectable but which are not carcinoma cell(s) or (lines). In a most preferred embodiment of the assay described herein, said carcinoma cell line is the above described MD4-MB-435S.

### EXAMPLE VII

### Tumor and Metastasis Formation of Parental, Wildtype and Mutant E-Cadherin Expressing Carcinoma Cells in a SCID Mouse Model

Abbreviations as employed in the following experimental example:

del 8 E-cadherin, E-cadherin with deletion of exon 8; del 9 E-cadherin, E-cadherin with deletion of exon 9; DMEM, Dulbecco's modified Eagle medium; FCS, fetal calf serum; n: number of animals; p8 E-cadherin, E-cadherin with point mutation in exon 8; h: hours; min: minutes; PBS, phosphate-buffered saline; SCID, severe combined immunodeficiency; wt E-cadherin: wild-type E-cadherin.

It was further investigated whether tumor-associated E-cadherin mutations impair the tumor-suppressive function of E-cadherin and influence metastasis formation in an orthotopic severe combined immunodeficiency (SCID) mouse model. The investigated E-cadherin mutations were in frame deletions of exons 8 (del 8) or 9 (del 9) and a point mutation in exon 8 (p8, D370A). Human MDA-MB-435S breast carcinoma cells stably expressing wild-type (wo or mutant E-cadherin were injected into the mammary fat pads of SCID mice, the E-cadherin-negative parental MDA-MB-435S cell line served as control. Tumor incidence was 100% for all cell lines. We found that mice transplanted with wt E-cadherin expressing MDA-MB-435S cells developed significantly smaller tumors than animals transplanted with the parental cell line. Mice transplanted with MDA-MB-435S cells expressing mutant E-cadherin variants (del 9 or p8) developed medium-size tumors, smaller than those developed in animals injected with the parental cell line, but larger than those developed in mice transplanted with wt E-cadherin expressing cells. The investigated E-cadherin mutations may impair the tumor-suppressive function of E-cadherin. Mice transplanted with the third mutant E-cadherin variant (del 8) developed tumors of approximately the same size as animals transplanted with wt E-cadherin expressing cells, suggesting that the effect of E-cadherin mutations on tumor development depends on the type of mutation. The dynamic of tumor formation was different between wt and del 8 E-cadherin expressing cells, since tumors obtained after transplantation of del 8 E-cadherin expressing MDA-MB-435S cells showed the highest proliferation rate and at the same time the highest amount of necrotic areas. Lung metastases were induced by all tested cell lines with a metastatic incidence of 80% for parental and wt E-cadherin expressing cells and from 60-100% for mutant E-cadherin expressing cells. Immunohistochemical analysis of E-cadherin expression in the tumors revealed a heterogeneous staining pattern, indicating loss or down-regulation of E-cadherin expression in some tumor cells. Metastases were completely negative for E-cadherin expression, suggesting that in this model metastases are formed when E-cadherin expression is lost. Taken together, loss of E-cadherin-mediated adhesion may promote tumor cell detachment from the primary tumor and dissemination of malignant cells to the lung. E-cadherin mutations significantly influenced tumor size and/or the mechanism of tumor formation. In contrast, the metastatic incidence was not significantly affected by the E-cadherin mutation status of the original cell lines.

Materials and Methods in this Example

Cell Cultivation

The human E-cadherin-negative mammary carcinoma cell line MDA-MB-435S (ATCC, Rockeville, USA) and the wild-type and mutant E-cadherin-cDNA transfected derivatives that were established by Handschuh et al. (29) were grown in Dulbecco's modified Eagle medium (DMEM, Life Technologies, Eggenstein, Germany) supplemented with 10% fetal calf serum (FCS, PAN Biotech, Aidenbach, Germany) and penicillin-streptomycin (50 IU/ml and 50 μg/ml; Life Technologies, Eggenstein, Germany) at 37° C. and 5% CO₂.

Flow Cytometry

Cells were harvested with versene and 5×10⁵ cells were incubated with 4 μg/ml monoclonal antibody directed to E-cadherin SHE78-7 (Alexis Deutschland, Grünberg, Germany) for 1 h on ice in PBS, washed with 0.1% sodium azide and 0.1% bovine serum albumine (Sigma, Deisenhofen, Germany) and stained with DTAF-conjugated anti mouse IgG (Jackson ImmunoResearch Laboratories, West Grove, USA) for 1 h on ice. Purified mouse IgG1 (Pharmingen, Heidelberg, Germany) was used as K immunoglobulin isotype control. Cells were analyzed on a Beckman Coulter Epics XL (Beckman Coulter, Krefeld, Germany).

Orthotopic Injection of MDA-MB-435S Transfectants into the Mammary Fat Pads of SCID Mice

SCID bg mice (Harlan Winkelmann, Borchen, Germany) were housed under pathogen-free conditions. Mice were anesthetized with fentanyl/dormitor/dormicum (0.05/0.5/1 mg/ml; Janssen-Cilag, Neuss, Germany; Pfizer, Karlsruhe, Germany; Hoffmann-La Roche, Grenzach-Wyhlen, Germany). Then a 5 mm incision was made in the skin to expose the mammary fat pad and 5×10⁶ cells were injected into the fad pad. The wound was closed with vicryl 6/0 (Johnson & Johnson, Brüssel, Belgium) and the animals were subcutaneously injected with 0.5 ml 5% glucose solution (B. Braun, Melsungen, Germany). Tumor growth (tumor length×width in mm²) was measured twice per week. Mice were sacrificed when the primary tumor had reached an approximate size of 10 mm after 38 and 46 days. Primary tumors were prepared and the tumor volumes in mm³ calculated. Primary tumors and organs were formalin-fixed and paraffin-embedded and analyzed histologically for metastases. Animals were handled according to the German animal protection guidelines.

Histological and Radiological Analysis

Four to five micrometer thick sections from the primary tumors, as well as other organs including heart, lungs, brain, thymus, colon, bone marrow, lymph nodes, spleen kidney and liver were cut and stained with hematoxylin and eosin (H&E). All sections were reviewed by two of the authors (M. K., L.Q-M). The size of the primary tumor and the metastases was measured with a grid ocular. All animals were radiologically analyzed in order to detect bone metastases.

Immunhistochemical Analysis

Immunohistochemistry was performed on an automated immunostainer (Ventana Medical Systems, Inc., Tucson, Ariz.) according to the company's protocols, with minor modifications. Formalin-fixed an paraffin-embedded del 8 and del 9 E-cadherin expressing MDA-MB-435S cells as well as sections from primary tumors and lungs were analyzed. After deparaffinization and rehydration, the slides were placed in a microwave pressure cooker in 0.01 mol/L citrate buffer (pH 6.0) containing 0.1% Tween 20 and heated in a microwave oven at maximum power for 30 min. The sections were cooled in Tris-buffered saline and washed in 3% goat serum for 20 min. The antibodies used included pan-cytokeratin (Sigma, #C2562), wild-type E-cadherin (Transduction Laboratories, BD Biosiences, Heidelberg, Germany, #C20820), and mutation specific del 8 and del 9 E-cadherin antibodies that were produced in our laboratory and reported elsewhere (25, 32). The proliferation rate was assessed with the monoclonal antibody against Ki-67 antigen (clone MiB1, Dako, Glostrup, Denmark). Appropriate positive controls were used to confirm the adequacy of the staining. A grid occular objective was used to count positive cells over 10 high-power fields (×40), and the percentage of positive cells was reported as 0 to 100%.

Statistical Analysis

The Mann-Whitney test was performed as described by Marcus et al (33). Significance was set to 5%. An exact 2-sided Chi-Square test was performed when appropriate.

Results

Flow cytometric investigation of the expression of wild-type and mutant E-cadherin

In the present study, the tumorigenic and metastatic properties of MDA-MB-435S mammary carcinoma cell transfectants expressing wt or mutant E-cadherin variants (del 8, del 9 and p8 E-cadherin) in SCID mice was compared. wt and mutant E-cadherin cDNAs were previously cloned from non-tumorous gastric mucosa or from diffuse-type gastric carcinomas, respectively (Handschuh, Oncogene 18 (1999), 4301-4312). Flow cytometric analysis of E-cadherin carried out with non-transfected, wt and mutant E-cadherin expressing MDA-MB-435S cells revealed that E-cadherin was expressed in all transfected cell lines in >95% of the cells (FIG. 15). Expression of wt and mutant E-cadherin in MDA-MB-435S cell transfectants was also demonstrated by immunoblot and immunofluorescence analyses (Handschuh, (1999), loc. cit.).

Orthotopic Injection of MDA-MB-435S Transfectants into the Mammary Fat Pads of SCID Mice

Parental and transfected MDA-MB-435S cell lines were injected into the mammary fat pads of 5-6 SCID mice per cell line. Tumor and metastasis were observed during 38-46 days after transplantation. All mice developed tumors in the mammary fat pad. Tumor growth was measured twice per week. An increase in tumor size was detectable for all cell lines (FIG. 16). The growth curves of tumors derived after inoculation of parental, del 9 and p8 E-cadherin expressing cells were not statistically different, while tumors obtained after injection of wt and del 8 E-cadherin expressing cells started at day 35 (wt-E-cadherin) or 28 (del 8E-cadherin) to grow significantly slower than those derived from parental cells (FIG. 16). Two animals per cell line were sacrificed at day 38 to investigate whether metastases were detectable (see below). The remaining mice were sacrificed at day 46. Primary tumors were prepared and tumor volumes were calculated (FIG. 16). Tumors obtained after transplantation of wt E-cadherin expressing MDA-MB-435S cells were significantly smaller than those obtained after injection of non-transfected parental cells (p=0.008) (FIG. 16). Different mutant E-cadherin variants had diverse effects on tumor formation: MDA-MB-435S cells expressing del 8 E-cadherin induced tumors of similar size as cells expressing wt E-cadherin, whereas MDA-MB-435S cells expressing del 9 and p8 E-cadherin induced tumors that were smaller than those obtained after transplantation of parental cells, but larger than those obtained after injection of wt E-cadherin expressing cells (FIG. 16). Tumors induced after transplantation of del 8 E-cadherin expressing MDA-MB-435S cells were significantly smaller than those obtained after injection of the parental cell line (p=0.008). In contrast, the differences in tumor sizes between tumors induced after injection of del 9 and p8 E-cadherin expressing MDA-MB-435S cells, and parental or wt E-cadherin expressing cells, did not reach statistical significance.

Metastases were identified only in lungs; their sizes ranged from few cells to large groups of neoplastic cells. Radiological analysis of all animals revealed that no bone metastases were detectable. E-cadherin-negative MDA-MB-435S cells caused lung metastases in 4 of 5 animals (80%). The incidence of lung metastasis was 60% for del 8 and del 9 E-cadherin and 100% for p8 E-cadherin. The differences in metastatic incidence in all investigated animals between the various cell lines was not statistically significant (p=0.477, Chi-Square Test). No significant differences in the number of lung metastases per animal were observable, neither after 38 nor after 46 days, nor when all data were combined. The areas of metastases were determined after 38 and 46 days. Also in this case, no significant differences were found between the various cell lines, neither after 38 nor after 46 days, nor when all data were combined (p=0.64).

Histological and Immunhistochemical Findings

Morphologically, all of the tumors looked similar and were composed of epithelial cells with large nuclei, prominent nucleoli, and abundant cytoplasm. Karyorrhexis and focal or confluent necrosis were frequently observed, mostly in the center of the tumors (FIG. 19A, B).

Expression of E-cadherin and cytokeratin was investigated in two mice per cell line in primary tumors and metastases (FIG. 17, Table I). Immunohistochemical analysis of E-cadherin in the tumors derived from parental MDA-MB-435S cells revealed complete absence of E-cadherin expression, whereas tumors derived from wt E-cadherin transfected cells expressed the protein in 20% of the neoplastic cells in a membranous pattern (FIG. 17A, B). The expression of E-cadherin in the remaining three groups of animals inoculated with del 8, del 9 and p8 E-cadherin expressing cell lines was diverse. E-cadherin expression was noticed only in 5% of the neoplastic cells in the tumors composed of del 8 E-cadherin cells. In contrast, tumors with del-9 and p8 E-cadherin mutations revealed a much higher positivity (del 9: 70% and 80%; p8: 60% and 80%) (FIG. 17 C-E). Cytokeratin was negative in primary tumors carrying the parental and wt E-cadherin expressing cells, whereas tumors with del 8, del 9 and p8 E-cadherin showed a faint membranous positivity in 5% or less of the neoplastic cells. All lung metastases were negative for E-cadherin (FIG. 17F, Table I) and cytokeratin, regardless of the cell line of origin.

In order to prove the success of the transfection, the cell blocks of the original cell lines and the tumors were stained with the specific antibodies against the two deletion variants (del 8 and del 9) (FIG. 18A-D). The cell line with del 8 E-cadherin revealed a 100% positivity with the corresponding antibody (FIG. 18A). In contrast, tumors derived from this cell line showed with only rare cells with a membranous positivity (FIG. 18B). The cell line with del 9 E-cadherin and the tumors derived from this cell line showed a membranous staining in the majority of the tumor cells with the specific antibody (FIG. 18C, D). The lung metastases were negative for both del 8 and del 9 E-cadherin antibodies (Table I).

The mean percentage of MiB1 positive cells varied from 15% to 70% in the different tumors (FIG. 19 A-G). Tumors derived from del 8 E-cadherin expressing MDA-MB-435S cells showed the highest proliferation rate as measured by MiB1 (mean 70%) (FIG. 19 B, E). Moreover, these tumors revealed the highest percentage of necrosis (>50%). In contrast, tumors derived from wt E-cadherin expressing MDA-MB-435S cells showed the lowest proliferation rate (mean 15%) (FIG. 19A, D), followed by the tumors derived from parental (mean 40%) (FIG. 5C), del 9 (mean 40%) (FIG. 19F) and p8 (mean 50%) (FIG. 19G) E-cadherin expressing MDA-MB-435S cells. Tumors obtained after inoculation of wt E-cadherin expressing MDA-MB-435S cells showed the lowest percentage of necrosis (30%).

The E-cadherin-catenin complex is critical for epithelial cell adhesion and maintenance of tissue integrity. Expressional abnormalities and mutational inactivation of E-cadherin are associated with a plurality of cancers and have been postulated to be implicated in tumor development and progression. Consistent with these findings, a tumor and invasion suppressor role of E-cadherin has been proposed. In this study, orthotopic transplantation of parental, wt and mutant E-cadherin expressing MDA-MB-435S cells was performed to investigate the effect of E-cadherin mutations on tumor and metastasis formation in SCID mice. Three major observations were made: First, primary tumor sizes depended on the E-cadherin expression and/or mutation status. Second, E-cadherin expression in tumors was heterogenous, indicating down-regulation or loss of E-cadherin. Third, lung metastases were completely negative for E-cadherin.

Tumor Size is Influenced by the E-Cadherin Expression and/or Mutation Status

Tumor volumes determined after 48 days revealed that expression of wt E-cadherin resulted in smaller tumor sizes in comparison to non-transfected parental cells, a finding that is consistent with the tumor-suppressive function of E-cadherin. Two of three mutant E-cadherin variants (del 9 and p8 E-cadherin) induced tumors that were larger than those obtained after injection of wt E-cadherin cadherin expressing cells. This result indicates that mutations of the E-cadherin gene cause a partial loss of the tumor-suppressive E-cadherin function.

However, the effect seems to depend on the type of E-cadherin mutation since del 8 E-cadherin expressing cells induced tumors of similar size as wt E-cadherin expressing cells. Tumors derived from del 8 E-cadherin expressing cells showed the highest percentage of Ki-67 positive cells (70%) of all investigated tumors, whereas tumors induced by wt E-cadherin expression cells revealed only a low percentage of Ki-67 positivity (15%). Conversely, the extent of necrosis and the percentage of apoptotic cells were elevated in tumors induced by del 8 E-cadherin expressing cells when compared with tumors derived from wt E-cadherin expressing cells. This result suggests that the dynamic of tumor growth is different in tumors derived from wt and del 8 E-cadherin expressing cells. Tumor growth induced by the highly proliferating del 8 E-cadherin expressing cells may be limited by insufficient supply with nutrients, leading to extensive necrosis and apoptosis.

The E-Cadherin Staining Pattern of Primary Tumors is Heterogeneous

Immunohistochemical analysis of E-cadherin expression revealed a heterogeneous staining pattern for all tumors. Tumors induced after inoculation of wt and del 8 E-cadherin expressing MDA-MB-435S cells showed few E-cadherin positive tumor cells (20% or 5%, respectively). Tumors with del 9 and p8 E-cadherin mutations revealed much higher positivities (del 9: 70% and 80%; p8: 60% and 80%). To prove that E-cadherin expression was detectable after formalin fixation and paraffin-embedding of the cells, cell blocks of the original cell lines were stained with mutation-specific E-cadherin antibodies. In accordance with previous results (Becker, Am. J. Pathol. 155 (1999), 1803-1809; Becker, J. Pathol. 197 (2002), 567-574), both del 8 and del 9 deletion variants were 100% positive with the respective antibodies. While the cell blocks were positive for E-cadherin, tumors derived from del 8 E-cadherin expressing cells showed occasional membranous staining. These data are in accordance with previous observations that del 8 E-cadherin was found to be localized perinuclear and only in punctuate areas at lateral membranous cell contacts in subconflucent MDA-MB-435S cells by laser scanning microscopy (Handschuh (1999), loc. cit.).

E-cadherin expression can be down-regulated by transcriptional repressors such as Snail, SIP1, and SLUG (Batlle, Nat. Cell Biol. 2 (2000), 84-89; Cano, Nat. Cell Biol. 2 (2000), 76-83; Comjin, Mol. Cell 7 (2001), 1267-278; Hemavathy, Gene 257 (2000), 1-12; Hajra, Cancer Res. 62 (2002), 1613-1618), by extracellular cleaving and shedding of E-cadherin mediated by matrix metalloproteinases (Noe, J. Cell Sci. 114 (2001), 111-118; Davies, Clin. Cancer Res. 7 (2001), 3289-3297) and by ubiquitination of the E-cadherin-catenin complex by Hakai, a c-Cbl-like protein (Fujita, Nat. Cell Biol. 4 (2002), 222-231). During apoptosis, cadherin-mediated cell-cell adhesion is disrupted by a mechanism that involves E-cadherin cleavage (Vallorosi, J. Chem. Biol. 275 (2000), 3328-3334; Steinhusen, J. Biol. Chem. 276 (2001), 4972-4980); the involved catalytic activities are caspases and metalloproteinases. The high number of apoptotic cells observed in tumors obtained after inoculation of del 8-E-cadherin expressing cells may be responsible for the low E-cadherin positivity, due to E-cadherin cleavage during apoptosis. Further observations point to an important role of the soluble tumor microenvironment for the presence of E-cadherin on tumor cells: plasmin has recently been shown to produce an E-cadherin fragment, thereby stimulating cancer cell invasion (40). In further experiments, it remains to be determined at which stage of tumor development E-cadherin is down-regulated. Since wt-E-cadherin expressing cells induce small-size tumors, we hypthesize that down-regulation of E-cadherin occurs as a late event. E-cadherin DNA methylation which frequently occurs in tumors (Strathdee, Cancer Biol. 12 (2002), 373-379), is unlikely to play a role in our model system, since the E-cadherin cDNA is expressed under the transcriptional control of the β-actin promoter, but not the native E-cadherin promoter.

Down-regulation of E-cadherin in nude mouse tumors has been observed by other authors, for instance in Harvey-murine-sarcoma-virus-transformed Madin Darby canine kidney cells (MDCK-ras) which produce malignant (i.e., invasive and metastatic) tumors in nude mice (Mareel, Int. J. Cancer 47 (1991), 922-928). Primary tumors as well as large metastases were heterogeneous, showing E-cadherin-positive well differentiated epithelial structures and E-cadherin-negative undifferentiated areas. Metastasis-derived cell cultures contained both E-cadherin-positive and E-cadherin-negative MDCK-ras-e cells during early passages in vitro. During further culture, however, they regained the homogeneous E-cadherin-positive characteristic of the original MDCK-ras-e cell line. In accordance with our own observatioons, the authors hypothesize that this result points to the existence of host factors, which are able to down-regulate E-cadherin expression, and they hypothesize that this down-regulation plays a basic role in invasion.

The Metastases are E-Cadherin Negative

Loss of E-cadherin-mediated adhesion may facilitate tumor cell detachment from the primary tumor and promote tumor cell dissemination. In the present study, all investigated cell lines induced lung metastases. The incidence of lung metastasis formation was 80% for parental and wt E-cadherin expressing cells, 60% for del 8 and del 9 E-cadherin expressing cells and 100% for p8 E-cadherin expressing cells. All metastases were completely negative for E-cadherin. Since all primary tumors showed heterogenicity for E-cadherin expression, the data suggest that loss or down-regulation of E-cadherin expression may promote tumor cell detachment from the primary tumor and dissemination of malignant cells.

The role of E-cadherin in the process of metastasis formation of MDA-MB-435S mammary carcinoma cells has been previously analysed (Meiners, Oncogene 16 (1998), 9-20). The mouse cDNA for E-cadherin was stably expressed in MDA-MB-435 carcinoma cells, and the altered cells were then injected into the mammary fat pads of nude mice, where they formed tumors, which spontaneously metastasized to the lungs. Expression of E-cadherin was inhibitory to metastasis formation. E-cadherin expression was detected throughout the primary tumors, but was completely absent in lung metastases. The authors concluded that induction of metastasis is detected when cell have lost epithelial characteristics.

Several studies show that E-cadherin function is critical in the induction and maintenance of cell polarity and differentiation, and its loss or down-regulation is associated with an invasive and poorly differentiated phenotype (Wijnhoven, Br. J. Surg. 87 (2000), 992-1005). The data of Pignatelli and collegues indicate that loss of membranous E-cadherin expression is associated with high grade and advanced stage in pancreatic cancer (Pignatelli, J. Pathol. 174 (1994), 243-248). The function of E-cadherin in preventing metastasis in tumour development is believed to be dependent on intracellular catenins. In another study (Bukholm, J. Pathol. 185 (1998), 262-266), the expression of alpha-, beta-, and gamma-catenins has been examined in a subset of the same tumours in order to evaluate their possible role in breast cancer metastasis. Tumour tissues from 90 primary breast carcinomas were immunostained for α-, β-, and γ-catenins. Reduced expression of each of the catenins alone failed to correlate with the probability of developing metastasis. However, when all of the four proteins (E-cadherin, α-, β-, and γ-catenin) were analysed as one group, a significant association was seen between reduction in immunoreactivity, of at least one of these four proteins and the presence of metastases. These results indicate that if one of these proteins is down-regulated, the function of the others in suppressing metastasis is altered. Taken together, an intact E-cadherin-catenin complex is required for the maintenance of normal tissue architechture and functional or expressional loss of E-cadherin promotes tumor growth. In the present study, we demonstrated that E-cadherin mutations impair the tumor-suppressive function of E-cadherin and/or alter the dynamics of tumor development. The tumors showed heterogeneous E-cadherin staining patterns, which indicates a loss or down-regulation of E-cadherin during tumor development. In accordance with this invention, loss of E-cadherin mediated cell adhesion promotes tumor cell dissemination, as suggested here, where the E-cadherin staining was absent in lung metastases.

### EXAMPLE VIII

### Time-Lapse Imaging of Tumor-Associated Mutant E-Cadherin Fused to Enhanced Green Fluorescent Protein During Cell Adhesion and Migration in Living Cells

In diffuse-type gastric carcinoma, mutations of the cell adhesion molecule E-cadherin are frequently observed. These mutations predominantly affect putative calcium binding motifs in the linker region between the second and third extracellular domains and have been shown previously to down-regulate cell adhesion and to up-regulate cell motility. The aim of the study was to investigate the localization of mutant E-cadherin during cell adhesion and migration. Accordingly, mutant E-cadherin with point mutation in exon 8 (p8, D370A) and wild-type (wIt) E-cadherin as control were C-terminally were fused to enhanced green fluorescent protein (EGFP) and expressed in human MDA-MB-435S mammary carcinoma cells. Time-lapse images were taken by laser scanning microscopy. The following results were obtained. First, spacial and temporal localization of the E-cadherin-EGFP fusion proteins was imaged during formation of cell contacts. Localization of the fusion protein p8-EcadEGFP differed significantly from that of wt E-cadherin fused to EGFP (wt-EcadEGFP). While wt-EcadEGFP was mainly localized at lateral membranes of contacting cells, p8 EcadEGFP was detectable occasionally and in a transient way at lateral contact sites. Instead, p8EcadEGFP was predominantly found in lamellipodia. Second, localization of the fusion proteins was determined during cell migration. Cell motility of p8-EcadEGFP expressing cells was enhanced as compared with low motile wt-EcadEGFP. p8EcadEGFP was predominantly found in lammelipodia during cell migration, while wt-EcadEGFP localized mainly at lateral cell contact sites. Third, inhibition of cell motility of p8-EcadEGFP expressing cells by application of a pharmacologic inhibitor of the epidermal growth factor receptor (EGFR) signalling pathway caused lateral membrane staining of p8EcadEGFP. Conversely, activation of EGFR by application of EGF induced motility and caused localization of wt-EcadEGFP in lamellipodia. These data show that the localization of mutant E-cadherin differs significantly from that of the wt molecule during cell adhesion and motility. The EGFR signalling pathway plays a regulatory role in the effect.

Materials and Methods for this Example

Construction of GFP-E-Cadherin cDNA

E-cadherin was fused to the N-terminus of enhanced green fluorescent protein by ligation of wild-type (wt) or mutant E-cadherin cDNA with point mutation mutation in exon 8 into vector pEGFP-N2 (Clontech, Palo Alto, USA, #6081-1). pEGFP-N2 encodes a red shifted GFP variant with an excitation maximum of 488 nm and an emission maximum of 507 nm. Isolation of wt E-cadherin cDNA from non-tumourous gastric mucosa or mutant E-cadherin cDNA from diffuse-type gastric carcinoma was described previously (Handschuh et al., 1999). Genomic mutations led to a point mutation in exon 8 changing codon GAT (position 370; clone HSECAD, Genbank/EMBL Z13009) to GCT. The stop codon at position 2741-2743 was mutated (TAG to TTA) to allow translation of the E-cadherin-EGFP fusion protein. The mutation was introduced into the wt and mutant E-cadherin expressing cDNAs using the Quick Change™ Site-Directed Mutagenesis Kit (Stratagene Europe, Amsterdam Zuidoost, Netherlands) following the instructions of the supplier. The correct sequences of all constructs was confirmed by sequencing.

Cell Cultivation and Transfection

The human E-cadherin-negative mammary carcinoma cell line MDA-MB-435S (ATCC, Rockeville, USA) was transfected either with vector pEGFP-N2 alone which encodes a neomycin-resistance cassette or with E-cadherin-EGFP-cDNA constructs using Transfast (Promega, Heidelberg, Germany, #2431). Stable clonal cell lines were established after selection with G418 and E-cadherin expression was tested by immunofluoresence and Western-Blot analysis. Transfectants were grown in Dulbecco's modified Eagle medium (DMEM, Life Technologies, Eggenstein, Germany) supplemented with 10% fetal calf serum (FCS, PAN Biotech, Aidenbach, Germany) and penicillin-streptomycin (50 IU/ml and 50 μg ml; Life Technologies, Eggenstein, Germany) at 370C and 5% CO₂. Genomic DNA was isolated from the stable transfectants and the correct sequences of wt or mutant p8E-cadherin was confirmed by sequencing.

Dynabeads Selection

E-cadherin-EGFP-expressing MDA-MB-435S clonal cell lines were selected with Dynabeads (CELLection™ Pan Mouse IgG kit, #115.19, Dynal Biotech, Hamburg, Germany). Antibody HECD-1 (Takara Shuzo Co., Shiga, Japan, distributed by Alexis Deutschland, Grünberg, Germany) was used for isolation of wt and p8 E-cadherin-EGFP expressing cells.

Time-Lapse Laser Scanning Microscopy

For time-lapse laser scanning microscopy, cells were cultivated overnight in a microscope-coupled incubation chamber (Zeiss, Jena, Germany) at 370C under 5% CO₂. Cells were seeded at a density of 1×10⁵ cells per 3.5-cm in collagen I-coated plates with a glass bottom which were purchased from MatTek Corporation (Ashland, Mass., USA). Uncoated plates were coated for 4 h at 37° C. with collagen I (100 μg/ml). EGFR kinase inhibitor Tyrphostin AG 1478 (Sigma, Deisenhofen, Germany) was used at a final concentration of 6.3 μM. EGF was used at a concentration of 100 ng/ml (Sigma). Phase contrast images were taken at 2-3 min intervals with an Axiovert laser scanning microscope LSM 510 (Zeiss) with lens PNF 63× (oil) and an argon laser at 488 nm with a transmission filter of 515 nm.

E-Cadherin Immunofluorescence Analysis

1×10⁵ cells were plated on glass coverslips in 6-well plates and cultivated for 3 days. Then, cells were fixed with methanol (−20° C.) and incubated for 2 h with anti-E-cadherin antibody HECD-1 (Alexis Deutschland, Grünberg, Germany; final concentration of 4 μg/ml). Next, cells were incubated for 30 min with a FITC-coupled secondary antibody (goat-anti-mouse IgG; Dianova, Hamburg, Germany).

Coverslips were mounted on slides using antifading-reagent (Molecular Probes, Leiden, Netherlands) and analyzed with an Axiovert laser scanning microscope.

Western Blot

For Western blot analysis, cells were lysed at a density of 80% in a 10 cm tissue culture dish with 500 μl L-CAM buffer (140 mM NaCl, 4.7 mM KCl, 0.7 mM MgSO₄, 1.2 mM CaCl₂, 10 mM Hepes pH 7.4, containing 1% (v/v) Triton-X-100 and 1 mM phenylmethylsulfonylfluoride. Proteins were separated by 10% SDS-polyacrylamide gel electrophoresis followed by transfer to a nitrocellulose membrane (Schleicher & Schuell, Dassel, Germany). Monoclonal antibodies against E-cadherin or GPF were purchased from Transduction Laboratories (anti-E-cadherin antibody #C20820 distributed by Dianova, Hamburg, Germany) or Clontech (GFP monoclonal antibody #8362-1 which reacts also with EGFP). For signal detection the enhanced chemoluminescence system (Amersham, Braunschweig, Germany) was used.

Temporal and Spatial Change in the Localization of Mutant E-cadherin During Cell Migration

The aim of the present study was the investigation of the temporal and spatial change in the localization of mutant E-cadherin during cell migration using a temperature and CO₂-controlled incubation chamber after application of inhibitors of the epidermal growth factor receptor (EGFR) and phosphatidyl inositol-3 kinase pathways, tyrphostin AG1478 and LY294002, and of the growth factor EGF. E-Cadherin-EGFP (EcadEGFP) was construced by fusion of the E-cadherin gene in front of the EGFP gene in the vector pEGFP-N2. MDA-MB-435S breast cancer cells which do not express endogenous E-Cadherin (Graff et al., 1995; Cano et al., 2000) were transfected with these Ecad-EGFP vectors using TransFast™ transfection reagent. As a control, MDA-MB-435S cells were transfected with pEGFP-N2 vector. Stable clones, expressing EGFP alone or the fusion protein were established. MDA-MB-435S transfectants expressing wild-type (wt)-E-cadherinEGFP and showing low locomotion activity, and highly motile cells expressing E-cadherin with point mutation in exon 8 (p8)-EcadEGFP were chosen. EGFP is a modified version of green fluorescent protein (GFP) which is characterized by enhanced fluoresence in comparison to GFP.

Sequence Analysis of the E-Cadherin Gene

In order to prove the expression and identity of E-cadherin-EGFP fusion protein in all transfectants, genomic DNA was isolated and the entire E-cadherin gene was sequenced. All clones revealed the expected mutation in the STOP codon (TAG [symbol] TTAG) (FIG. 20) which is necessary to prevent a translational stop and indicates that the fusion protein can be expressed. All p8-EcadEGFP transfectants expressing mutant E-cadherin carried the expected point mutation in exon 8 (GAT [symbol] GCT, D370A) (FIG. 20). No additional mutations in the E-cadherin sequence were detected.

Detection of EcadEGFP—Expression by Western Blot Analysis

Expression of EGFP, wt- and p8-EcadEGFP was shown by Western blot analysis. Cellular lysates of cells expressing EGFP or the fusion protein, were analysed using antibodies directed against E-Cadherin or GFP. Using both antibodies, a 150 kDa band corresponding to the fusion protein was detected in EcadEGFP expressing cell lines. This band migrates slower than un-tagged wt E-cadherin (120 kD) which is present in the control lysates (FIG. 21, A+B). A 30 kD band corresponding to EGFP was detectable in lysates from vector-transfected cell lines (FIG. 21, B).

Besides Western Blot analysis, FACS analysis was performed to show the expression of E-cadherin at the cell surface. Non-transfected MDA-MB-435S cells (MDA) served as negative control. Vector-transfected MDA-MB-435S cells (EGFP) revealed the strongest signal intensity (FIG. 22). Transfectants expressing the fusion proteins (wt-EcadEGFP, p8-EcadEGFP) showed strong signals (FIG. 22). While wt-EcadEGFP transfectants revealed a small peak, p8-EcadEGFP transfectants showed a broad fluoresence spectrum which partially overlapped with the signal of control cells.

Immunfluorescence Analysis

Previous investigations from our laboratory had shown that E-cadherin influences the organisation of the actin cytoskeleton (Handschuh et al., 1999, Luber et al., 2000). To investigate whether these changes are also detectable in EcadEGFP expressing cells, the transfectants were stained with rhodamine-conjugated phalloidin. In accordance with our previous observations, wt-EcadEGFP expressing cells formed colonies with tight cell cell contacts. wt-EcadEGFP was predominantly localized at cell contact sites and only in small amounts in the cytoplasm (FIG. 23, A). The actin cytoskeleton was organized as a circumferential ring and parallel actin fibers were detectable (FIG. 23, B). In contrast, p8-EcadEGFP transfectants were characterized by reduced cell cell contacts with only few contact sites and gaps in the monolayer (FIG. 23, D). Cells at the edge of the clone formed lamellipodiae and showed a tendency to separate from the clone. p8-EcadEGFP was localized predominantly at cell cell contact sites and in the cytoplasm as well as around the nucleus and in lamellipodiae (FIG. 4, D). Occasionally, a circumferential actin belt was visible. Actin fibers were disorganized and did not reveal the parallel organization as present in wt-EcadEGFP expressing cells. These results were in accordance with previous observation by Handschuh et al. (1999) and showed that EGFP does not impair the E-cadherin function in actin cytoskeleton organization.

Next, the influence of mutant EcadEGFP on the intracellular localization of α-catenin was investigated and the results obtained by Luber et al. (2000) were confirmed. Besides its function as outside-in signalling molecule, β-catenin binds to the cytoplasmic domain of E-cadherin and connects the cell adhesion molecule with the actin cytoskeleton (Ozawa et al., 1990; Aberle et al., 1994; Hülsken et al., 1994; Rimm et al., 1995). This interaction is a prerequisite for cell adhesiveness and suppression of cell motility and invasion (Frixen et al., 1991; Vleminckx et al., 1991). First, localization of EGFP and β-catenin were investigated in vector-transfected MDA-MB-435S cells. In these cells, the EGFP signal was diffusely distributed in the cytoplasm, and predominantly localized around the nucleus (FIG. 24, A). The reason for this observation is that the protein is small enough to enter the nucleus. No enhanced fluorescence was detectable at cell cell contact sites. β-catenin was localized predominantly at cell cell contact sites and partially in the nucleus in vector-transfected cells (FIG. 24, B).

Superposition of both fluorescence chanels revealed no co-localization between EGFP and β-catenin. Consequently, EGFP mediated effects on β-catenin localization in vector-transfected cells were ruled out. β-catenin localization at the cell membrane can be mediated by N-Cadherin which is expressed in MDA-MB-435S cells (Handschuh et al., 2002). Similar results were obtained for the localization of β-catenin in wt-EcadEGFP expressing cells (FIG. 24, E+F). In these cells, β-catenin was localized at cell cell contact sites and was associated with N-Cadherin or wt-EcadEGFP and partially in the nucleus. Perinuclearly localized wt-EcadEGFP may be associated with the golgi apparatus. In p8EcadEGFP expressing cells, signals from mutant EcadEGFP and from β-catenin were diffusely distributed in the cells with enhanced signal intensity at the cell cell contact sites (FIG. 24, G+H). Perinuclear p8EcadEGFP localization was stronger as in wt-EcadEGFP transfectants.

Taken together, the results of the immunofluoresence analysis demonstrate a similar 13-catenin distribution in MDA-MB-435S cells as previously described by Luber et al. (2000) and shows that the EGFP-tag has no detectable effect on the subcellular localization of β-catenin.

In Vitro Motility Studies in the Incubation Chamber

To investigate the temporal localization of mutant E-cadherin during cell migration, EcadEGFP transfected cells were cultivated in an incubation chamber coupled with a Zeiss Axiovert 100 microscope. Cells transfected with the vector pEGFP-N2 Vektor or the different EcadEGFP constructs (wt-EcadEGFP and p8-EcadEGFP), were seeded on collagen I coated dishes with glas bottom. Films were started the next day 30 min. to 2 h after addition of medium with EGF (100 ng/ml) or Tyrphostin (6,3 μM).

Localization of Wt- and Mutant E-Cadherin and Motile Behaviour of Cells

wt-ECadEGFP-transfected cells showed low motility (4,8-9,9 μm/h). Cell paths were short (19-39,4 μm, FIG. 27). Cells mostly rotated around their own axis or showed movements at the cellular edges. At low density, cells formed small colonies and maintained cell-cell contacts. Dividing cells re-attached to the extracellular matrix immediately after division and formed new contacts to neighbouring cells. wt-EcadEGFP was localized at cell cell contact sites and not at free clone edges. At higher magnification, structures resembling E-cadherin bundles were visible (FIG. 25, A). At the edges of connecting cells, the contact was even stronger and E-cadherin accumulations were detectable (FIG. 25, B). p8-EcadEGFP expressing MDA-MB-435S cells (21,4-102,8 μm) were higher motile than wt-EcadEGFP transfected cells (5,3-25,7 μm/h; p=0,023; Mann-Whitney-Test). Motile cells showed a strong tendency to separate from each other and formed only transiently cell cell contacts (FIG. 26). During migration, lamellipodiae and strong membrane ruffling was visible. The direction of cell movement was random. After cell division, cells separate from each other instead of forming contacts (FIG. 32). EGFP-tagged p8-E-cadherin was detectable at the edge of cells during migration, in lamellipodiae and at cell cell contact sites. Moreover, it was localized perinuclear and in the cytoplasm (FIG. 26+32).

The EGFR Inibitor Tyrphostin AG1478 Induces Localization of Mutant E-Cadherin At Cell Cell Contact Sites

To investigate the influence of EGFR on the localization of wt and p8-EcadEGFP, cell motility was investigated in the presence of Tyrphostin AG1478. In wt-EcadEGFP transfected cells, Tyrphostin AG1478 significantly influenced cell motility (1,3-8,8 μm/h) and cell paths (15,1-35,2 μm) (FIG. 27+28; p=0,016; Kruskal Wallis Test). Morphologically, cells were characterized by a small and round shape and enhanced cell cell contact but the localization of wt-EcadEGFP was not significantly changed (FIG. 30). In contrast, the migratory phenotype of p8-EcadEGFP expressing cells was strongly influenced by Tyrphostin AG1478. Cell motility was reduced (5,7-20 μm/h; p=0,199; Kruskal Wallis Test) and cells resembled morphologically wt-EcadEGFP transfected cells. The changes of the direction of cell movement were not as abrupt as before and cell paths were shorter (22,8-80,3 μm, FIG. 27+28). Membrane ruffling decreased (FIG. 33). After cell division, cells migrated toward each other instead of separating from one another. p8-EcadEGFP was not only localized in lamellipodiae and diffusely distrubuted in the cytoplasm, but was also localized perinuclear and at cell cell adhesion sites (FIG. 33).

EGF induces Localization of Wt-EcadEGFP in Lamellipodiae

To investigate whether EGF induces the motility of wt-EcadEGFP expressing MDA-MB-435S transfectants, cells were starved overnight (0,5% (v/v) FCS in DMEM). Starved cells were spindle-shaped with reduced cell cell contacts and formed no colonies. After addition of EGF, cells resembled p8-EcadEGFP expressing cells, formed lamellipodiae and showed membrane ruffling. wt-EcadEGFP was localized at cell cell contact sites and at the cell edges and in lamellipodiae (FIG. 31). Cell paths increased (15,1-62,4 μm; 3,8-15,6 μm/h; FIG. 27 +28), cell cell contacts formed only for short time periods. EGF had only minor effects on the motility of p8-EcadEGFP transfected MDA-MB-435S cells (FIG. 34, (p=0,364; Kruskal Wallis Test). p8-EcadEGFP was detectable at the cell edges and in lamellipodiae and at cell cell contact sites. Cell motility and cell paths did not increase (19,7-97,2 μm; 5,0-24,3 μm/h; FIG. 27+28).

Taken together, our results demonstrate that the localization of E-cadherin after application of Tyrphostin AG1478 or EGF depends on the mutation status of E-cadherin. wt-EcadEGFP which is normally localized at cell cell contact sites, is found in the cytoplasm, the pericnuclear region and in lamellipodia after application of EGF. In contrast, the EGFR inhibitor Tyrphostin AG1478 has no influence on the localization of wt-EcadEGFP. p8-EcadEGFP is normally localized at cell edges, in lamellipodiae and at transiently formed cell cell contact sites. EGF had no influence on p8EcadEGFP localization, while Tyrphostin AG1478 caused that p8EcadEGFP was localized at cell cell contacts and perinuclear.

### References of Example VIII

Aberle H, Bauer A, Stappert J, Kispert A, Kemler R (1997). Beta-catenin is a target for the ubiquitin-proteasome pathway. EMBO J. 16: 3797-3804.
Adams C L, Chen Y T, Smith S J, Nelson W J (1998). Mechanisms of epithelial cell-cell adhesion and cell compaction revealed by high-resolution tracking of E-cadherin-green fluorescent protein. J. Cell Biol. 142: 1105-1119.
Cano A, Perez-Moreno M A, Rodrigo I, Locascio A, Blanco M J, del Barrio M G, Portillo F and Nieto M A (2000). The transcription factor snail controls epithelial-mesenchymal transitions by repressing E-cadherin expression. Nat Cell Biol. 2: 76-83.
Davenport D, Nichol J A C (1955). Luminescence in Hydromedusae. Proceedings of the Royal Society, Series B 144: 399-411.
Frixen U H, Behrens J, Sachs M, Eberle G, Voss B, Warda A, Lochner D, and Birchmeier W (1991). E-cadherin-mediated cell-cell adhesion prevents invasiveness of human carcinoma cells. J. Cell Biol. 113: 173-185.
Graff J R, Herman J G, Lapidus R G, Chopra H, Xu R, Jarrard D F, Isaacs W B, Pitha P M, Davidson N E, and Baylin S B (1995). E-cadherin expression is silenced by DNA hypermethylation in human breast and prostate carcinomas. Cancer Res. 55: 5195-5199.
Handschuh G, Candidus S, Luber B, Reich U, Schott C, Oswald S, Becke H, Hutzler P, Birchmeier W, Höfler H, and Becker K-F (1999). Tumour-associated E-cadherin mutations alter cellular morphology, decrease cellular adhesion and increase cellular motility. Oncogene 18: 4301-4312.
Heim R, Prasher D C, Tsien R Y (1994). Wavelength mutations and posttranslational autoxidation of green fluorescent protein. Proc Natl Acad Sci U S A. 91: 12501-12504.
Hülsken J, Birchmeier W, Behrens J (1994). E-cadherin and APC compete for the interaction with beta-catenin and the cytoskeleton. J Cell Biol. 127: 2061-2069.
Lino R, Koyama I, Kusumi A (2001). Single molecule imaging of green fluorescent proteins in living cells: E-cadherin forms oligomers on the free cell surface. Biophys J. 80: 2667-2677.
Jefferson R A, Kavanagh T A, Bevan M W (1987). GUS fusions: beta-glucuronidase as a sensitive and versatile gene fusion marker in higher plants. EMBO J, 6: 3901-3907.
Luber B, Candidus S, Handschuh G, Mentele E, Hutzler P, Feller S, Voss J, Hofler H, Becker K F (2000). Tumor-derived mutated E-cadherin influences beta-catenin localization and increases susceptibility to actin cytoskeletal changes induced by pervanadate. Cell Adhes Commun. 7: 391-408.
Morin, J. G., and Hastings, J. W. (1971) Energy Transfer in a Bioluminescent System. J. Cell Physiol. 77:313-317.
Morise H, Shimomura 0, Johnson F H, Winant J (1974). Intermolecular energy transfer in the bioluminescent system of Aequorea. Biochemistry. 13: 2656-2662.
Ozawa M, Ringwald M, Kemler R (1990). Uvomorulin-catenin complex formation is regulated by a specific domain in the cytoplasmic region of the cell adhesion molecule. Proc Natl Acad Sci USA. 87: 4246-4250.
Rimm D L, Sinard J H, Morrow J S (1995). Reduced alpha-catenin and E-cadherin expression in breast cancer. Lab Invest. 72: 506-512.
Sanger F, Nicklen S, Coulson A R (1977). DNA sequencing with chain-terminating inhibitors. Proc Natl Acad Sci USA 74: 5463-7.
Shiozaki H, Oka H, Inoue M, Tamura S, Monden M (1996). E-cadherin mediated adhesion system in cancer cells. Cancer 77:1605-1613. Review.
Vleminckx K, Vakaet L Jr, Mareel M, Fiers W, van Roy F (1991). Genetic manipulation of E-cadherin expression by epithelial tumor cells reveals an invasion suppressor role. Cell 66: 107-119.

### EXAMPLE IX

### Epidermal Growth Factor Receptor Immunohistochemical Reactivity at the Invasion Front Correlates with Poor Survival in Gastric Adenocarcinoma from Mexican Patients

The aim of this example was to determine epidermal growth factor receptor (EGFR) expression in gastric adenocarcinoma by standardized immunohistochemistry using an EGFR detection system and to correlate EGFR expression with clinical features and patient survival.

For this purpose, EGFR expression was investigated in paraffin sections of resection specimens of 89 gastric carcinomas. Membrane staining of EGFR was evaluated in the neoplastic cells and graded using a semiquantitative score (0-3+). Staining of neoplastic cells was negative in 47 cases (52.8%), weak in 17 tumors (19.1%, score 1+), moderate in 16 adenocarcinomas (18.0%, score 2+) and strong in 9 neoplasms (10.1%, score 3+). EGFR reactivity was very heterogeneous, frequently showing completely negative up to 3+positive areas. A correlation was found between EGFR reactivity score and distant metastases (p=0.002) or clinical stage (p=0.033), but not between EGFR score and histotype, tumor invasion, perigastric lymph node status or residual disease. EGFR score 0/1+ was significantly associated with an increase in patient survival, when compared to score 2+/3+ (p=0.0006). The presence of EGFR reactive cells in muscle layer and subserosa was associated with a decrease in patient survival (p=0.0004). Cox regression analysis revealed that the prognosis was associated with the EGFR reactivity score, EGFR reactive neoplastic cells in mucosa (p=0.019), muscularis or subserosa (p=0.001), submucosa, muscularis or subserosa (0.002), distant metastases (p=0.0001) and residual disease (p=0.012) in an univariate analysis. A multivariate analysis revealed that EGFR positive cells in muscularis or subserosa (p=0.004), distant metastases (p=0.016) and residual disease were significantly correlated with a decrease in survival (0.012). Accordingly, it can be shown that EGFR reactivity at the deep tumor invasion front is correlated with poor survival in gastric adenocarcinoma.

Materials and Methods for this Example

Patient Selection

Patients with total gastrectomy operated with the diagnosis of adenocarcinoma since 1982 to 2001 in the Instituto Nacional de Ciencias Medicas y Nutricion Salvador Zubiran, with available clinical information and follow-up were considered. The clinical variables obtained from the charts were age at diagnosis, gender, survival time and cause of death. A blinded review of all the slides was made by pathologists and a diagnosis according to Laurén's classification of gastric adenocarcinoma was made (Laurén, Acta Pathol. Microbiol. Scand. 64 (1965), 31-49). Mestizo Mexican patients with available paraffin material and a morphologic diagnosis of poorly differentiated intestinal, mixed or diffuse-type adenocarcinoma in which UICC staging criteria (Spiessl, (1992), loc. cit.) could be reproduced, were included.

EGFR Immunohistochemistry

A hematoxylin/eosin stained section was obtained and reviewed for morphologic confirmation and two consecutive sections were mounted on charged slides for immunohistochemistry. Immunostainings for EGFR were performed using the Dako EGFRpharmDx™ assay detection system (Dako Corporation, Carpinteria, Calif.) which recognizes a 170 kDa transmembrane receptor encoded by the human HER1 gene. The manual staining protocol was precisely followed, and no substitutions were made. After dewaxing in fresh xylene, 100% ethanol, 95% ethanol and 70% ethanol (4 baths each), the slides were placed in a humid chamber for proteolytic digestion with proteinase K solution (100 μl for 5 min), and quenching of endogenous peroxidase for 5 min. The primary antibody was incubated for 30 min followed by 30 min incubation with labelled polymer, and DAB localization of the positive cells. Counterstain was made with hematoxylin followed by 10 slide dips in a bath containing 37 mmol/l amonia water. In every run control slides were included which were provided to validate the performance of the reagents of the Dako EGFRpharmDX™ assay detection kit. The control slides contained sections of pelleted, formalin-fixed, paraffin-embedded cell line HT-29 with a moderate level of EGFR protein expression (positive control, 1HC staining score of the cell pellet is 2.5+0.5) and of the EGFR negative CAMA-1 cell line (negative control, score 0).

EGFR Reactivity Evaluation

Membrane staining was evaluated in the neoplastic cells and quantified and graded as recommended in the detection kit:

| | |
|---|---|
| 0 score | No staining observed, or membrane staining in <10% |
| | neoplastic cells. Negative. |
| 1+ score | Weak complete and/or incomplete membrane staining |
| | in >10% neoplastic cells. Positive. |
| 2+ score | Moderate complete and/or incomplete membrane staining |
| | in >10% neoplastic cells. Positive. |
| 3+ score | Strong complete and/or incomplete membrane staining |
| | in >10% neoplastic cells. Positive. |

Localization and intensity of reactivity was evaluated for mucosa, submucosa and deeper zones (muscle layer and subserosa). Statistical analyses were performed using Fisher's exact, K and X² tests when appropriate. Kaplan-Meier survival time analysis was used to correlate EGFR reactivity, localization of positive cells (surface or deep), pT, pN and pM status with clinical evolution. Cox regression analysis was performed correlating EGFR reactivity, localization of positive cells, and stage with prognosis. A two sided p value less than 0.05 was considered to be statistically significant.

Results

Clinicopathological Features

The clinicopathological features of 89 Mestizo Mexican patients with gastric cancer are shown in appended Table 1. The mean and median of the patient ages at the time of diagnosis were 57.8 or 60.0 years, respectively, with a range of 14-86 years and a standard deviation of 15.2 years. 44 patients (49.4%) were female, 45 patients (50.6%) were male. The gastric cancer histotype was classified according to Laurén: 36 cases (40.4%) were of poorly differentiated intestinal type, 49 tumor samples (55.1%) were of diffuse type and 4 cases (4.5%) were of mixed type, containing both intestinal and diffuse components. The stages (UICC) were IB in 1 patient (1.1%), II in 25 patients (28.1%), IIIA in 20 patients (22.5%), IIIB in 14 cases (15.7%) and IV in 29 cases (32.6%). The residual disease status was R0 in 69 cases (77.5%) and R1 in 20 cases (22.5%).

EGFR Score of Reactivity

89 slides from gastric carcinomas were stained with the EGFR immunohistochemical detection system. Membrane staining was evaluated in the neoplastic cells and quantified and graded as recommended in the detection kit (appended Table 2). 47 cases (52.8%) were negative or reactive in <10% of neoplastic cells (score 0). Complete and/or incomplete membrane staining in >10% of neoplastic cells was weak in 17 tumors (19.1%, score 1+), moderate in 16 adenocarcinomas (18.0%, score 2+, FIG. 37) and strong in 9 neoplasms (10.1%, score 3+). The percentage of EGFR reactive cells per case was also evaluated, without considering the staining intensity. 26 cases (29.2%) were completely EGFR negative. 21 cases (23.6%) showed reactivity in <10% of neoplastic cells, 30 cases (33.7%) were reactive in 10-50% of tumor cells and 12 cases (13.5%) were positive in >50% of neoplastic cells. Nerve and muscle cells served as reactive internal control. Normal gastric mucosa showed no EGFR staining. EGFR reactivity frequently showed a striking variability in the tumor tissue. In some cases, only few tumor cells were highly reactive (score 3+), while the rest of the tumor showed low reactivity or complete absence of EGFR expression.

EGFR Score and its Correlation with Clinicopathological Features and Morphology

The EGFR score of reactivity was correlated with clinicopathological features and morphology (appended Table 3). EGFR reactivity score 2+/3+ was present in 9/36 intestinal type, in 1/4 mixed type and in 15/49 diffuse type gastric carcinomas. There was no statistically significant association between EGFR score and histotype according to Laurén (p=0.201). In 0/8 cases with tumor invasion stage pT 2 and in 25 cases with stage pT 3-4, EGFR reactivity score 2+/3+ was detectable. EGFR score and depth of tumor invasion were not significantly correlated (p=0.304). 3/21 cases with perigastric lymph node status pN0 and 22/68 cases with status pN1-2 showed EGFR score 2+/3+. There was no correlation between EGFR score and perigastric lymph node status (p=0.313). EGFR reactivity score 2+/3+ was present in 16/71 cases without distant metastases and in 9/18 cases with metastases. The association between distant metastases and EGFR reactivity score was statistically significant (p=0.002). In 17/69 cases with residual disease status R0 and in 8/20 cases with status R1 EGFR score 2+/3+ was present. There was no significant association between EGFR score and residual disease (p=0.406). EGFR reactivity score 2+/3+ was detectable in 2/26 cases with clinical stage I-II and in 22 cases with stage III-IV. The correlation between EGFR score and clinical stage was statistically significant (p=0.033).

Influence of EGFR Score and Percentage of EGFR Reactive Neoplastic Cells on Survival

Kaplan Meier survival time analysis was used to correlate EGFR score and percentage of reactive neoplastic cells with patient survival. Survival time of patients with EGFR scores 0/1+ was significantly increased when compared with EGFR score 2+/3+(p=0.0006, FIG. 36). The log rank test statistical analysis indicates a global p value 0.0083. When the percentage of EGFR reactive cells was correlated with patient survival, no EGFR reactivity or reactivity in <10% cells resulted in increased patient survival when compared with EGFR reactivity in 10-50% or >50% cells. This trend was observable, although the result did not reach statistical significance (global p value 0.0688). The mean and median of the overall patient follow-up were 21.3 month or 12.0 month, respectively, with a range of 1-173 months and a standard deviation of 28.8 month.

Distribution of EGFR Reactive Neoplastic Cells and Association of EGFR Reactivity at the Invasion Front with Survival

Localization and intensity of reactivity was evaluated for mucosa, submucosa and deeper zones (muscle layer and subserosa). The presence of EGFR reactive cells at the invasion front was significantly associated with a decrease in patient survival (global p value 0.0004, FIG. 38).

Cox regression analysis was performed to correlate EGFR reactivity score, percentage and localization of positive cells, stage, distant metastases and residual disease status with prognosis (appended Table 4, univariate all patients). EGFR reactivity score was associated with the length of survival (p=0.003). In contrast, the percentage of EGFR positive cells was not correlated with patient survival (p=0.071), although a trend was detectable. Furthermore, a signifcant association with survival was observed for positive neoplastic cells in mucosa (p=0.019), muscularis or subserosa (p=0.001), submucosa, muscularis or subserosa (p=0.002), distant metastases (p=0.001) and residual disease (p=0.012). A multivariate analysis revealed that EGFR positive cells in muscularis or subserosa (p=0.004), distant metastases (p=0.016) and residual disease (p=0.039) were significantly correlated with a decrease in survival (appended Table 4).

Kaplan Meier survival time analysis was used to correlate residual disease status with patient survival. Survival time of patients with status R0 was significantly increased when compared with status R1 (global p value 0.0028, FIG. 5).

EGFR as Prognostic Marker in Gastric Cancer

Data reported in this example finding that EGFR reactivity score has been identified as a prognostic indicator in gastric cancer is in accordance with several studies which have demonstrated that EGFR expression correlates with poor prognosis (Nicholson, Eur. J. Cancer 27 Sppl. 4 (2001), 9-15). Recently, the relationship between EGFR expression and cancer prognosis was investigated based on the analysis of literature data of more than 200 studies published between 1985 and 2000 (Nicholson (2001), loc. cit.). It was found that EGFR expression was a strong prognostic indicator in cancers of the head and neck, ovary, cervix, bladder, and esophagus, and that EGFR expression correlated with reduced recurrence-free and overall survival in 70% of studies included in the literature search. In gastric, breast, endometrial and colorectal cancers EGFR expression was associated with poor survival in 52% of the included studies, while in non-small-cell-lung cancer only 30% of studies showed such a correlation between EGFR expression and survival. For gastric cancer, co-expression of EGFR and its ligands EGF or TGF-a was found to be correlated with a decrease of survival or the relapse-free survival interval (Yasui, Int. J. Cancer 41 (1988), 211-217; Yonemura, Oncology 49 (1992), 157-161; Tokunaga, Cancer 75 (1995), 1418-1425). Amplification (Tsugawa, Oncology 55 (1998), 475-481; Hirono, Oncology 52 (1995), 182-188) or expression of EGFR (Yasui (1988), loc. cit.) was correlated with advanced clinical stage and the presence of lymph node metastasis (Yasui, Cancer Res. 48 (1988), 137-141; Lida, Oncology 52 (1995), 189-195).

EGFR Positivity at the Deep Invasion Front

In the present study, the presence and staining intensity of EGFR reactive cells were evaluated in mucosa, submucosa and at the deep invasion front in muscle layer and subserosa after exclusion of patients with early cancer in muscosa and submucosa. The localization of EGFR reactive cells in muscle layer and subserosa was associated with a decrease in patient survival which indicates that EGFR positivity at the deep invasion front is critical in determining the patient's outcome. In a recent study using the same technique in colonic adenocarcinoma, positivity at the invasion front also showed the strongest correlation with survival duration as well as with EGFR positivity of lymph node and liver metastases (Goldstein, Cancer 92 (2001), 1331-1346). Increased EGFR expression at the most invasive parts of carcinomas has also been reported for oral squamous cell carcinomas (Bankfalvi, J. Pathol. 198 (2002), 343-351). These data support the hypothesis that the invasive front of carcinomas is the most critical area for prognostication (Goldstein (2001), loc. cit.; Bryne, Anticancer Res. 18 (1998), 4757-4764; Bankfalvi, J. Oral Pathol. Med. 29 (2000), 291-298).

Heterogeneity of EGFR Expression

EGFR reactivity showed a marked intratumoral heterogeneity, frequently showing a range of variations of completely negative up to 3+positive neoplastic cells within an individual case. EGFR staining heterogeneity was also observed for colonic adenocarcinoma (Goldstein (2001), loc. cit.). These observations argue for an up-regulation of EGFR expression in later stages of tumor progression. Different mechanisms, like autocrine stimulation by growth factors, genetic instability or transcriptional disregulation may be considered. With regard to anti-EGFR therapy, the impact of EGFR heterogeneity on the therapeutic response has to be clarified. It may also be of importance in the evaluation of small tumor samples, e.g. pretherapeutic endoscopic samples.

### EXAMPLE X

### Correlative Analysis of Epidermal Growth Factor Receptor Expression and Immunohistochemical Reactivity with Mutation-Specific E-Cadherin Antibodies

Abbreviations Used in this Example

CI: confidence intervall; del 8 E-cadherin, E-cadherin with deletion of exon 8; del 9 E-cadherin, E-cadherin with deletion of exon 9.

This example X was undertaken to determine epidermal growth factor receptor (EGFR) expression in gastric adenocarcinoma by standardized immunohistochemistry and immunohistochemical reactivity with mutation-specific E-cadherin antibodies recognizing E-cadherin lacking exon 8 (del 8) or 9 (del 9). EGFR and del 8 or del 9 E-cadherin expression were examined in paraffin-embedded resection specimens of 92 gastric carcinomas from Mexican Mestizo patients. The gastric cancer histotype according to Laurén was intestinal type in 37 cases (40.0%), diffuse type in 51 tumor samples (55.0%) and mixed type in 4 cases (5.0%). EGFR expression was investigated using a standardized detection system. Membrane staining of EGFR was evaluated in the tumor cells and graded using a semiquantitative sore (0-3+). EGFR expression was observed in 43 patients (47.0%). Staining of neoplastic cells was weak in 17 tumors (19.5%, score 1+), moderate in 17 adenocarcinomas (18.5%, score 2+), strong in 9 neoplasms (10.0%, score 3+), and negative in 49 cases (53.0%). In an univariate analysis, EGFR reactivity score 2+/3+(p=0.002) and stage III/IV (p=0.02) were significantly associated with prognosis. Multivariate analysis using Cox's proportional hazard model revealed that EGFR reactivity score 2+/3+(p=0.012) and stage III/IV (p=0.028) were significantly associated with poor prognosis. del 8 or del 9 E-cadherin reactivity in combination with EGFR reactivity and stage further decreases the survival prognosis. Since del 8 or del 9 E-cadherin reactivity is predominantly found in diffuse and mixed type gastric carcinoma, further analysis was performed with 55 cases, after exclusion of intestinal type tumor patients. In an univariate analysis, stage III/IV (p=0.007) was significantly associated with prognosis. Multivariate analysis using Cox's proportional hazard model revealed that stage III/IV (p=0.005) was significantly associated with poor prognosis. del 8 or del 9 E-cadherin reactivity in combination with stage further decreases the survival prognosis.

Conclusion: Our data indicate that in combination with EGFR score 2+/3+ and stage III/IV, del 8 or del 9 E-cadherin reactivity contributes to poor prognosis in gastric carcinoma. When only diffuse and mixed type gastric carcinomas are considered, stage III/IV is the most important prognostic factor and additional del 8 or del 9 E-cadherin further decreases the patient's survival chances.

Materials and Methods

Patient Selection

Patients with total gastrectomy operated with the diagnosis of adenocarcinoma since 1982 to 2001 in the Instituto Nacional de Ciencias Medicas y Nutricion Salvador Zubiran, with available clinical information and follow-up were considered. The clinical variables obtained from the charts were age at diagnosis, gender, survival time and cause of death. A blinded review of all the slides was made by pathologists and a diagnosis according to Laurén's classification of gastric adenocarcinoma was made. Mestizo Mexican patients with available paraffin material and a morphologic diagnosis of poorly differentiated intestinal, mixed or diffuse-type adenocarcinoma in which UICC staging criteria could be reproduced, were included.

EGFR Immunohistochemistry

A hematoxylin/eosin (H&E) stained section was obtained and reviewed for morphologic confirmation and two consecutive sections were mounted on charged slides for immunohistochemistry. Immunostainings for EGFR were performed using the Dako EGFRpharmDx™ assay detection system (Dako Corporation, Carpinteria, Calif.) which recognizes a 170 kDa transmembrane receptor encoded by the human HER1 gene. The manual staining protocol was precisely followed, and no substitutions were made. After dewaxing in fresh xylene, 100% ethanol, 95% ethanol and 70,% ethanol (4 baths each), the slides were placed in a humid chamber for proteolytic digestion with proteinase K solution (1001 μl for 5 min), and quenching of endogenous peroxidase for 5 min. The primary antibody was incubated for 30 min followed by 30 min incubation with labelled polymer, and DAB localization of the positive cells. Counterstain was made with hematoxylin followed by 10 slide dips in a bath containing 37 mmol/l amonia water. In every run control slides were included which were provided to validate the performance of the reagents of the Dako EGFRpharmDx™ assay detection kit. The control slides contained sections of pelleted, formalin-fixed, paraffin-embedded cell line HT-29 with a moderate level of EGFR protein expression (positive control, 1HC staining score of the cell pellet is 2.5+0.5) and of the EGFR negative CAMA-1 cell line (negative control, score 0).

EGFR Reactivity Evaluation

Membrane staining was evaluated in the neoplastic cells and quantified and graded as recommended in the detection kit:

| | |
|---|---|
| 0 score | No staining observed, or membrane staining in <10% |
| | neoplastic cells. Negative. |
| 1+ score | Weak complete and/or incomplete membrane staining |
| | in >10% neoplastic cells. Positive. |
| 2+ score | Moderate complete and/or incomplete membrane staining |
| | in >10% neoplastic cells. Positive. |
| 3+ score | Strong complete and/or incomplete membrane staining |
| | in >10% neoplastic cells. Positive. |

E-Cadherin Immunohistochemical Analysis

Immunohistochemistry was performed on an automated immunostainer (Ventana Medical Systems, Inc., Tucson, Ariz.) according to the company's protocols, with minor modifications. Formalin-fixed and paraffin-embedded sections from primary tumors were analyzed. After deparaffinization and rehydration, the slides were placed in a pressure cooker in 0.01 mol/L citrate buffer (pH 6.0) containing 0.1% Tween 20 and heated in a microwave oven at maximum power for 30 min. The sections were cooled in Tris-buffered saline and washed in 3% goat serum for 20 min. The antibodies used included anti-E-cadherin antibody AEC (clone 36, Tranduction laboratories, Lexington, N.Y., dilution 1:1000) and mutation specific del 8 and del 9 E-cadherin antibodies that were produced in our laboratory and reported elsewhere (Becker et al, 1999; 2002). Appropriate positive controls were used to confirm the adequacy of the staining. AEC reactivity was defined as normal (membranous), atypic (partial membran staining, cytoplasmic or heterogenous staining) or negative staining. del 8 and del 9 E-cadherin was considered positive when membranous staining was observed.

Statistical Analysis

Statistical analyses were performed using Fisher's exact, K and X² tests when appropriate. Kaplan-Meier survival time analysis was used to correlate EGFR reactivity, localization of positive cells (surface or deep), pT, pN and pM status with clinical evolution. Cox regression analysis was performed correlating EGFR reactivity, localization of positive cells, and stage with prognosis. A two sided p value less than 0.05 was considered to be statistically significant.

Results

Clinicopathological Features

The clinicopathological features of 92 Mestizo Mexican gastric cancer patients are shown in Table 5. Median patient age at the time of diagnosis was 58 years (range of 14-86 years). 47 patients (51%) were female, 45 patients (49%) were male. Classification of gastric cancer histotype according to Laurén was poorly differentiated intestinal type in 37 cases (40%), diffuse type in 51 tumor samples (55%), mixed type with intestinal and diffuse components in 4 cases (5%). The stages according to the UICC classification were IA, B in 3 patients (3%), II in 25 patients (27%), IIIA in 20 patients (22%), IIIB in 14 cases (15%) and IV in 30 cases (33%).

EGFR Score of Reactivity and del 8 or del 9 G-Cadherin Reactivity

Gastric carcinomas were stained with standardized EGFR immunohistochemical detection systems. Membrane staining was evaluated in the neoplastic cells and quantified and graded as recommended in the detection kits (Table 6). For EGFR expression, 49 cases (53.0%) were negative or reactive in <10% of neoplastic cells (score 0). Complete and/or incomplete membrane staining in >10% of neoplastic cells was weak in 17 tumors (18.5%, score 1+), moderate in 17 adenocarcinomas (18.5%, score 2+) and strong in 9 neoplasms (10.0%, score 3+). Nerve and muscle cells were reactive and served as reactive internal control. Normal gastric mucosa was negative for EGFR expression. EGFR reactivity showed a broad variability with a range from few positive tumor cells surrounded from a majority of negative neoplastic cells to equal EGFR expression in almost all tumor cells.

del 8 or del 9 E-cadherin expression was investigated using mutation-specific anti-E-cadherin antibodies (Becker et al, 1999; 2002, loc. cit.). del 8 or del 9 E-cadherin staining was observed in 10/92 cases (10.9%).

Influence of EGFR Score, del 8 or del 9 E-Cadherin Reactivity, and Stage on Survival

In an univariate analysis, the correlation of EGFR reactivity score, del 8 or del 9 E-cadherin reactivity, and stage with prognosis was investigated (Table 7). A statistically significant association was found between the length of survival and EGFR reactivity score 2+/3+(p=0.002) as well as stage III/IV (p=0.02).

A multivariate analysis using Cox's proportional hazard model revealed that EGFR score 2+/3+(p=0.012) and stage III/IV (p=0.028) were significantly correlated with a decrease in survival. The presence of del 8 or del 9 E-cadherin reactivity is disadvantageous for the patients. The relative risk to dye in patients with EGFR score 2+/3+ and del 8 or del 9 E-cadherin reactivity, was elevated 2.454 fold×2.142 fold (5.256 fold). Consequently, del 8 or del 9 E-cadherin reactivity is an additional risk factor.

Influence of EGFR Score, del 8 or del 9 E-Cadherin Reactivity and Stage on Survival in diffuse and mixed type Gastric Carcinomas

Previous observations have demonstrated the presence of E-cadherin mutations in diffuse and mixed type, but not in intestinal type gastric carcinomas (Becker et al, 1994, loc. cit.). In the present study with tumors from Mexican patients, del 8 or del 9 E-cadherin reactivity was found in 6 diffuse, 3 mixed and 1 intestinal type gastric carcinomas (Gamboa-Dominguez et al, in preparation).

EGFR reactivity score, del 8 or del 9 E-cadherin reactivity, and stage were correlated with prognosis in diffuse and mixed type cases (Table 8, univariate all patients). Only stage III/IV was statistically correlated with patient survival (p-0.007). A multivariate analysis using Cox's proportional hazard model revealed that del 8 or del 9 E-cadherin reactivity was more important for prognosis in diffuse and mixed type gastric carcinomas than EGFR score 2+/3+, this trend was observable (p=0.174). del 8 or del 9 E-cadherin reactivity is an additional risk factor. Stage III/IV (p=0.005) was significantly correlated with a decrease in survival.

Influence of del 8 or del 9 E-Cadherin Reactive Neoplastic Cells or EGFR Score and Stage on Survival

Kaplan Meier method was used to correlate stage and del 8 or del 9 E-cadherin reactivity with patient survival (FIG. 40). In the presence of del 8 or del 9 E-cadherin reactivity, survival time of patients in stage I/II or III/IV was significantly decreased. The log rank test indicates a global p value 0.009.

Kaplan Meier method was used to investigate the correlation between EGFR expression and stage with patient survival (FIG. 41). In the presence EGFR reactivity, survival time of patients in stage III/IV was decreased. The log rank test indicates a global p value 0.0326. Taken together, for patients in stage I/II or II/IV, survival prognosis is poorer in the presence of EGFR or del 8 or del 9 E-cadherin reactivity. Taken together, the data suggest that expression of EGFR as well as presence of del 8 or del 9 E-cadherin reactivity in combination with tumor stage contribute to poor prognosis. Therefore, treatment of patients with these two abnormalities is highly recommended.

Tables relating to the examples:

TABLE 1
Clinicopathologic features of 89 patients with gastric cancer.

| Age | years |
|---|---|
| Mean | 57.8 |
| Median | 60.0 |
| Standard deviation | 15.2 |
| Range | 14-86 |

| | n | % |
|---|---|---|
| Gender | | |
| Female | 44 | 49.4 |
| Male | 45 | 50.6 |
| Histotype (Laurén) | | |
| Intestinal | 36 | 40.4 |
| Diffuse | 49 | 55.1 |
| Mixed | 4 | 4.5 |
| Stage (UICC) | | |
| IB | 1 | 1.1 |
| II | 25 | 28.1 |
| IIIA | 20 | 22.5 |
| IIIB | 14 | 15.7 |
| IV | 29 | 32.6 |
| Residual Disease | | |
| R0 | 69 | 77.5 |
| R1 | 20 | 22.5 |

TABLE 2
EGFR reactivity in 89 patients with gastric cancer.

| | |
|---|---|
| n | % |

EGFR score of reactivity

| | | |
|---|---|---|
| 0 | 47 | 52.8 |
| 1+ | 17 | 19.1 |
| 2+ | 16 | 18.0 |
| 3+ | 9 | 10.1 |

Percentage of EGFR positive cells

| | | |
|---|---|---|
| 0% | 26 | 29.2 |
| <10% | 21 | 23.6 |
| 10-50% | 30 | 33.7 |
| >50% | 12 | 13.5 |

TABLE 3
EGFR score of reactivity and its correlation with clinicopathological
features and morphology in 89 Mexican Mestizo patients with gastric
cancer.

| EGFR score | | | | |
|---|---|---|---|---|
| 0 | 1+ | 2+ | 3+ | total |

Histotype (Laurén)

| | | | | | |
|---|---|---|---|---|---|
| Intestinal | 23 | 4 | 7 | 2 | 36 |
| Mixed | 1 | 2 | 0 | 1 | 4 |
| Diffuse | 23 | 11 | 9 | 6 | 49 |
| | | p = 0.201 | | | |

Tumor invasion

| | | | | | |
|---|---|---|---|---|---|
| pT 2 | 7 | 1 | 0 | 0 | 8 |
| pT 3-4 | 40 | 16 | 16 | 9 | 81 |
| | | p = 0.304 | | | |

Perigastric lymph node status

| | | | | | |
|---|---|---|---|---|---|
| pN0 | 13 | 5 | 1 | 2 | 21 |
| pN1-2 | 34 | 12 | 15 | 7 | 68 |
| | | p = 0.313 | | | |

Distant metastases

| | | | | | |
|---|---|---|---|---|---|
| pM0 | 40 | 15 | 7 | 9 | 71 |
| pM1 | 7 | 2 | 9 | 0 | 18 |
| | | p = 0.002 | | | |

Residual disease

| | | | | | |
|---|---|---|---|---|---|
| R0 | 39 | 13 | 10 | 7 | 69 |
| R1 | 8 | 4 | 6 | 2 | 20 |
| | | p = 0.406 | | | |

Clinical stage (UICC)

| | | | | | |
|---|---|---|---|---|---|
| I-II | 19 | 5 | 1 | 1 | 26 |
| III-IV | 28 | 12 | 15 | 8 | 63 |
| | | p = 0.033 | | | |

TABLE 4
Analysis of prognostic factors in gastric carcinomas
univariate

| | Significance (p value) |
|---|---|
| EGFR reactivity score | 0.003 |
| Percentage of EGFR reactive neoplastic cells | 0.071 |
| EGFR reactive cells in mucosa | 0.019 |
| EGFR reactive cells in submucosa | 0.124 |
| EGFR reactive cells in muscularis or subserosa | 0.001 |
| EGFR reactive cells in submucosa, | 0.002 |
| muscularis or subserserosa | |
| Stage III-IV | 0.064 |
| Distant metastases | 0.0001 |
| Residual Disease | 0.012 |

multivariate: Cox proportional hazard model
in a stepwise forward fashion

| | | 95% CI for relative risk | | |
|---|---|---|---|---|
| | Significance | relative | | |
| | (p value) | risk | Lower | Upper |
| EGFR reactive cells in | 0.004 | 2.679 | 1.373 | 5.224 |
| muscularis or subserosa | | | | |
| Distant metastases | 0.016 | 2.583 | 1.190 | 5.607 |
| Residual Disease | 0.039 | 2.057 | 1.037 | 4.082 |

CI: confidence intervall

TABLE 5
Clinicopathologic features of 92 patients with gastric cancer

| | |
|---|---|
| Age (median), years | 58 (Range 14-86) |

| | n | % |
|---|---|---|
| Gender | | |
| Female | 47 | 51 |
| Male | 45 | 49 |
| Histotype (Laurén) | | |
| Intestinal | 37 | 40 |
| Diffuse | 51 | 55 |
| Mixed | 4 | 5 |
| Stage (UICC) | | |
| IA, B | 3 | 3 |
| II | 25 | 27 |
| IIIA | 20 | 22 |
| IIIB | 14 | 15 |
| IV | 30 | 33 |

TABLE 6
EGFR score of reactivity in 92 patients with gastric adenocarcinoma

| EGFR Score | n | % |
|---|---|---|
| 0 | 49 | 53.0 |
| 1+ | 17 | 18.5 |
| 2+ | 17 | 18.5 |
| 3+ | 9 | 10.0 |

TABLE 7
Analysis of prognostic factors in gastric carcinomas
univariate

| | Significance (p value) |
|---|---|
| EGFR reactivity score 2+/3+ | 0.002 |
| del 8 or del 9 E-cadherin reactivity | 0.853 |
| Stage III/IV | 0.020 |

multivariate: Cox proportional hazard model
in a stepwise forward fashion

| | | 95% CI for Relative Risk | | |
|---|---|---|---|---|
| | Significance | Relative | | |
| | (p value) | Risk | Lower | Upper |
| EGFR reactive score 2+/3+ | 0.012 | 2.454 | 1.220 | 4.934 |
| del 8-, del 9 E-cadherin | 0.125 | 2.142 | 0.809 | 5.671 |
| reactivity | | | | |
| Stage III/IV | 0.028 | 2.241 | 1.096 | 5.002 |

CI: confidence intervall

TABLE 8
Analysis of prognostic factors in diffuse and mixed type
gastric carcinomas
univariate

| | Significance (p value) |
|---|---|
| EGFR reactivity score 2+/3+ | 0.106 |
| del 8 or del 9 E-cadherin reactivity | 0.690 |
| Stage III/IV | 0.007 |

multivariate: Cox proportional hazard model
in a stepwise forward fashion

| | | 95% CI for Relative Risk | | |
|---|---|---|---|---|
| | Significance | Relative | | |
| | (p value) | Risk | Lower | Upper |
| del 8-, del 9 E-cadherin | 0.174 | 1.948 | 0.745 | 5.094 |
| reactivity | | | | |
| Stage III-IV | 0.005 | 3.687 | 1.492 | 9.114 |

CI: confidence intervall

1. Use of (an) EGF receptor antagonist(s)/inhibitor(s) for the preparation of a pharmaceutical composition for the prevention, amelioration or treatment of gastric carcinomas.

2. The use of claim 1 wherein said gastric carcinoma is a diffuse gastric carcinoma.

3. The use of claim 1 or 2 for inhibiting the motility of tumor cells in a subject suffering from said carcinomas.

4. The use of any one of claims 1 to 3 wherein the carcinoma cells of the patients suffering from said carcinomas do not comprise an overexpression of EGF receptor.

5. The use of any one of claims 1 to 4 wherein the cells derived from said carcinomas comprise at least one mutation in the β-catenin signal transduction pathway.

6. The use of any one of claims 1 to 4 wherein cells derived from said carcinoma comprise a mutation in E-cadherin.

7. The use of claim 6 wherein said E-cadherin mutation is selected from the group consisting of a full or partial deletion of exon 8, a full or partial deletion of exon 9, a full or partial deletion of exon 10 and one or more point mutations.

8. The use of any one of claims 1 to 7, whereby said EGF receptor antagonist(s)/inhibitor(s) inhibits/inhibit the motility or metastasis formation of cells comprising at least one mutation in the β-catenin signal transduction pathway.

9. A method for the treatment of gastric carcinomas as defined in any one of the claims 1 to 8 comprising the administration of (an) EGF-receptor antagonist(s)Anhibitor(s) to a subject in need of such a treatment.

10. The use of any one of claims 1 to 8 or the method of claim 9 whereby the EGF-receptor antagonist/inhibitor is selected from the group consisting of an anti-EGF-receptor antibody or a derivative or a fragment thereof, an EGF-toxin or immunotoxin, antisense oligonucleotides specifically interacting with nucleic acid molecules encoding EGFR, siRNA or RNAi directed against EGFR, ribozymes specifically interacting with EGFR nucleic acid molecules or tyrosine kinase inhibitors.

11. The use or the method of claim 10, wherein said EGF-toxin is conjugated to Pseudomonas exotoxin A or a truncated version thereof whereby said EGF-toxin is fused to genistein.

12. The use or the method of claim 10, wherein said tyrosine kinase inhibitor is tyrphostin AG1478, ZD-1839, OSI-774, PKI-166, PD 158780, CPG 59326 or CI-1033.
